(19) United States
(12) Patent Application Publication
(10) Pub. No.: US 2023/0338402 A1
(43) Pub. Date: Oct. 26, 2023

(54) TREATMENT REGIMEN FOR CANCER USING IMMUNOMODULATION

(71) Applicant: BioXcel Therapeutics, Inc., New Haven, CT (US)

(72) Inventor: Vincent J. O'NEILL, New Haven, CT (US)

(21) Appl. No.: 17/797,388

(22) PCT Filed: Feb. 5, 2021
(86) PCT No.: PCT/US2021/016764

§ 371 Date: Aug. 3, 2022

Related U.S. Application Data
(60) Provisional application No. 63/139,594, filed on Jan. 20, 2021.
(60) Provisional application No. 63/107,053, filed on Oct. 29, 2020.
(60) Provisional application No. 63/105,679, filed on Oct. 26, 2020.
(60) Provisional application No. 63/092,039, filed on Oct. 15, 2020.
(60) Provisional application No. 63/088,038, filed on Oct. 6, 2020.
(60) Provisional application No. 63/069,405, filed on Aug. 24, 2020.
(60) Provisional application No. 63/029,066, filed on May 22, 2020.
(60) Provisional application No. 62/971,548, filed on Feb. 7, 2020.

(51) Int. Cl. A61K 31/69; C07K 16/28; A61K 39/00; A61P 35/04

(52) U.S. Cl. CPC A61K 31/69; C07K 16/2818; A61K 39/464411; A61P 35/04; A61K 2039/505; A61K 2039/545; A61K 2039/542

(57) ABSTRACT

The present disclosure provides a regimen for treating a subject afflicted with prostate cancer by administering Talabostat or a pharmaceutically acceptable salt thereof and Pembrolizumab, wherein Talabostat or a pharmaceutically acceptable salt thereof is administered at a dose of about 0.3 mg twice daily on one or more days of a treatment cycle. The disclosure further relates to a treatment regimen or a method comprising said combination to treat one or more solid tumors.

### CROSS REFERENCE TO RELATED APPLICATIONS

The present application claims the benefit of priority to U.S. Provisional Application No. 62/971,548, filed on Feb. 7, 2020, U.S. Provisional Application No. 63/029,066, filed on May 22, 2020, U.S. Provisional Application No. 63/069,405, filed on Aug. 24, 2020, U.S. Provisional Application No. 63/088,038, filed on Oct. 6, 2020, U.S. Provisional Application No. 63/092,039, filed on Oct. 15, 2020, U.S. Provisional Application No. 63/105,679, filed on Oct. 26, 2020, U.S. Provisional Application No. 63/107,053, filed on Oct. 29, 2020, and U.S. Provisional Application No. 63/139,594, filed on Jan. 20, 2021, each of which are hereby incorporated by reference in their entirety.

### FIELD

The present disclosure relates to a treatment regimen for treating cancer comprising administering Talabostat or a pharmaceutically acceptable salt thereof and Pembrolizumab.

### BACKGROUND

Cancer is a multistep process that begins with minor pre-neoplastic changes, which may progress to neoplasia, the neoplastic lesions possibly developing an increasing capacity for invasion, growth, metastasis, and heterogeneity. Current therapies for the treatment of cancer involve surgery, hormonal therapy, radiation therapy, chemotherapy and immunotherapy. Immunotherapy for the treatment of cancer has evolved alongside our improved understanding of the immune system. In particular, an appreciation of the ability of cancer cells to subvert the antitumor immune response has provided a rationale for the development of novel immunotherapies that target immune checkpoints responsible for tumor cells escaping detection and destruction by the immune system.

Such immune escape mechanisms are mediated either directly by the tumor cells or by the tumor microenvironment. Tumor cells are known to express membrane proteins, secreted products, enzymes, anti-inflammatory cytokines, and chemokines to produce changes in their genome that aid in immune evasion and immune inhibition. At the same time, a key role is played by the tumor microenvironment.

Immune checkpoint molecules such as PD-1, PD-L1, CTLA-4 are cell surface signaling receptors that play important roles in modulating the T-cell response in the tumor microenvironment. Tumor cells have been shown to utilize these checkpoints to their benefit by up-regulating their expression and activity. Therefore, immune checkpoint inhibitors have been developed which can unleash the immune system's cancer-destroying properties. Recent discoveries have identified immune checkpoints or targets like PD-1, PD-L1, PD-L2, CTLA4, TIM3, LAG3, CCR4, OX40, OX40L, IDO, and A2AR as proteins responsible for immune evasion, acting as “brakes” of the immune system. Specific immune checkpoint inhibitors, including antibodies against CTLA-4, the PD-1 receptor and its ligand PD-L1 have produced impressive results in the clinic, leading to FDA approvals for Yervoy® (Ipilimumab; CTLA-4 antagonist), Opdivo® (Nivolumab; PD-1 antagonist) and KEYTRUDA® (Pembrolizumab; PD-1 antagonist) in multiple tumor indications and with on-going clinical trials in many more.

Unfortunately, checkpoint inhibitors suffer from several limitations. Only a minority of patients treated with checkpoint inhibitors exhibit robust anti-tumor responses, and most responses are partial and temporary. Often patients initially respond, but then relapse due to the emergence of resistant pathways, which mainly occur due to the generation by the tumor cells of a non-immune permissive microenvironment.

The combination of the two checkpoint inhibitors, Ipilimumab and Nivolumab, have shown an increase in the response rate in melanoma patients from 11% and 32% seen with the respective monotherapy to 60% with the combination. Unfortunately, this combination has the significant drawback of high toxicity related to an excessive immune response, leading to pneumonitis, hepatitis, colitis and other immune related disorders.

Talabostat also known as PT-100 (Val-boroPro; L-valinyl-L-boroproline), was originally developed by Point Therapeutics, during 2000 to 2007. It is an orally available synthetic selective inhibitor of dipeptidyl peptidases like FAP and DPP8 and DPP9. The stereoisomer of the Talabostat molecule disclosed in the U.S. Pat. No. 6,825,169 while its oral formulation such as tablet, capsule, lozenges is disclosed in the U.S. Pat. No. 7,265,118.

Talabostat plays an important role in immune evasion and regulates both innate and/or acquired immunity. However, Talabostat has been reported to exhibit a number of side effects at therapeutically effective doses, with the most common adverse events being edema/peripheral swelling, hypotension, hypovolemia, and dizziness. These reported adverse events, as well as insufficient primary and secondary outcomes in certain cancer clinical trials, have led to the limited use of Talabostat as an anti-cancer agent.

In U.S. Patent Application Publication No. 2017/0266280A1 (incorporated herein by reference in its entirety for all purposes), the novel discovery that the combination of a selective dipeptidyl peptidase (DPP) inhibitor such as Talabostat with an immune checkpoint inhibitor is effective in treating cancer is disclosed. However, there is still an unmet need for improved cancer therapies and regimens. This invention meets that need.

### SUMMARY

The present disclosure is based on the discovery that the combination of Talabostat or a pharmaceutically acceptable salt thereof and Pembrolizumab in a specific treatment regimen comprising administering Talabostat or a pharmaceutically acceptable salt thereof at a dose of about 0.3 mg twice daily on one or more days of a treatment cycle, and Pembrolizumab on day 1 of a treatment cycle is a safe and effective therapy to treat subjects afflicted with prostate cancer or an advanced solid tumors. The treatment regimen is well tolerated and produces less severe adverse effects relative to a subject with same cancer that is administered 0.6 mg once daily dose of Talabostat.

Thus, in an aspect, the present disclosure provides a regimen or a method for treating prostate cancer in a subject in need thereof, comprising administering to said subject, Talabostat or a pharmaceutically acceptable salt thereof at a dose of about 0.3 mg twice daily on one or more days of a treatment cycle and an effective amount of Pembrolizumab on day 1 of a treatment cycle.

In some embodiments, the method comprises at least one administration cycle (e.g. 1, 2, 3, 4, 5, 6 or more cycles), and each treatment cycle is of about 21 days. In some embodiments, said treatment is administered for at least 12 weeks, or at least 24 weeks. In some embodiments, the patient achieves a complete response within 2-4 weeks after treatment.

In some embodiments, the separate pharmaceutical formulations of Talabostat or a pharmaceutically acceptable salt thereof and Pembrolizumab are administered to the subject at relevant times, and in suitable amounts, during one or more treatment cycles of about 21 days, to maximize their combined immunotherapeutic effect. In some embodiments, Talabostat or a pharmaceutically acceptable salt thereof is administered orally (e.g. as a tablet formulation) at a dose of 0.3 mg twice a day. In some embodiments, Pembrolizumab is administered intravenously at a total dose of about 200 mg.

In some embodiments, Talabostat or a pharmaceutically acceptable salt thereof is administered orally at a dose of about 0.2 mg twice daily on day 1-7 of the first treatment cycle followed by about 0.3 mg twice daily on day 8-14 of the first treatment cycle.

In some embodiments, Talabostat or a pharmaceutically acceptable salt thereof is administered orally at a dose of about 0.3 mg twice daily on day 1-3 of the first treatment cycle followed by rest period of 4 days then about 0.3 mg twice daily on day 8-11 of the first treatment cycle,

In some embodiments, the subject afflicted with prostate cancer has experienced dose-limiting adverse effects (or DLT) with a single 0.6 mg daily dose of Talabostat.

In some embodiments, the prostate cancer is selected from the group consisting of small cell neuroendocrine prostate cancer; (SCNC), neuroendocrine prostate cancer (NEPC), treatment emergent neuroendocrine prostate cancer (tNEPC), castration resistant prostate cancer (CrPC), metastatic castration resistant prostate cancer (mCrPC) and adenocarcinoma type prostate cancer). In a preferred embodiment, the prostate cancer is adenocarcinoma type prostate cancer. In another embodiments, the prostate cancer is small cell neuroendocrine prostate cancer. In another embodiments, the prostate cancer is treatment emergent neuroendocrine prostate cancer (tNEPC). In another embodiment, the prostate cancer is metastatic. In another embodiment, the prostate cancer is at an advanced stage.

In another aspect, the present disclosure provides methods for treating one or more solid tumors (e.g. an advanced solid tumor) in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle.

In some embodiments, the solid cancer is selected from the group consisting of castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma and uveal melanoma.

In some embodiments, the subject was not previously treated with PD-1/PD-L1 or CTLA-4 antibodies (or treatment naïve).

In some embodiments, the subject has relapsed with PD-1/PD-L1 or CTLA-4 antibodies (or treatment experienced).

In some embodiments, the subject has progressed with PD-1/PD-L1 or CTLA-4 antibodies.

In some embodiments, the subject administered with such a treatment regimen achieves a 50% or greater prostate-specific antigen (PSA) decline from baseline by week 12 of treatment.

In some embodiments, the subject administered with such a treatment regimen achieves a stable disease response or better, as measured by RECIST 1.1.

In some embodiments, the subject administered with such a treatment regimen achieves a partial disease response or better, as measured by RECIST 1.1.

In some embodiments, the subject administered with such a treatment regimen achieves a complete disease response or better, as measured by RECIST 1.1.

In an aspect, the present disclosure provides a method of enhancing an innate immune response in a subject afflicted with prostate cancer (e.g. small cell neuroendocrine prostate cancer; (SCNC), neuroendocrine prostate cancer (NEPC), treatment emergent neuroendocrine prostate cancer (tNEPC), castration resistant prostate cancer (CrPC), metastatic castration resistant prostate cancer (mCrPC), adenocarcinoma type prostate cancer), the method comprising administering to said subject, Talabostat or a pharmaceutically acceptable salt thereof at a dose of about 0.3 mg twice daily on one or more days of a treatment cycle and Pembrolizumab on day 1 of the treatment cycle, wherein the enhanced innate immune response is associated with increased tumoricidal natural killer cells and macrophages, as well as the activity of NK cells and CD8+ T cells. In a particular aspect, the enhanced innate immune response is associated with suppression of T-regulatory cells.

In another aspect, the disclosure provides a method of enhancing an innate immune response in a subject afflicted with one or more solid tumors (e.g. an advanced solid tumor), comprising administering to said subject, Talabostat or a pharmaceutically acceptable salt thereof at a dose of about 0.3 mg twice daily on one or more days of a treatment cycle (e.g. day 1 to 14) and Pembrolizumab on day 1 of the treatment cycle, wherein the enhanced innate immune response is associated with increased tumoricidal natural killer cells and macrophages, as well as the activity of NK cells and CD8+ T cells. In a particular aspect, the enhanced innate immune response is associated with suppression of T-regulatory cells.

In a further aspect, the present disclosure provides a method of enhancing proinflammatory cytokine release in a subject afflicted with prostate cancer, the method comprising administering to said subject, Talabostat or a pharmaceutically acceptable salt thereof at a dose of about 0.3 mg twice daily on one or more days of a treatment cycle and Pembrolizumab on day 1 of the treatment cycle, wherein the enhanced proinflammatory cytokine release is associated with activation of Natural Killer (NK) cells and T cells resulting in increased antitumor response.

In one embodiment, the proinflammatory cytokines are one or more of IL-18, IL-1β and IFN-γ. In another embodiment, the subject experiences an increase in pro-inflammatory cytokines relative to a subject that is administered a single 0.6 mg daily dose of Talabostat.

In some embodiments, a time-dependent increase in IL-18 levels with maximum increase on day 14 of continuous dosing is observed.

In another aspect, the subject is afflicted with one or more solid tumors (e.g. an advanced solid tumor).

In a further aspect, the present disclosure provides a method of reducing the treatment related adverse effects (TRAEs) in a subject afflicted with prostate cancer, comprising administering to said subject, Talabostat or a pharmaceutically acceptable salt thereof at a dose of about 0.3 mg twice daily on one or more days and Pembrolizumab on day 1 of a treatment cycle. In a particular aspect, the subject experiences lesser treatment-related adverse events (TRAEs) or (no DLT) relative to a subject with same cancer that is administered a single 0.6 mg once daily dose of Talabostat.

In some embodiments, the TRAEs are selected from one or more of hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, rhabdomyolysis, myalgia, constipation, hypocalcemia, neutropenia, febrile neutropenia, anemia, leukopenia, pancytopenia, and lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest.

In other embodiment, the subject experiences no TRAEs. In other embodiment, the subject experiences TRAEs that are consistent with cytokine release.

In one specific embodiment, the prostate cancer is selected from small cell neuroendocrine prostate cancer (SCNC), neuroendocrine prostate cancer (NEPC), treatment emergent neuroendocrine prostate cancer (tNEPC), castration resistant prostate cancer (CrPC), metastatic castration resistant prostate cancer (mCrPC), adenocarcinoma type prostate cancer. In another embodiment, the solid cancer is selected from the group comprising castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma.

Other features, objects, and advantages of the disclosure will be apparent from the description and drawings, and from the claims.

### BRIEF DESCRIPTION OF THE DRAWINGS

FIG. 1: shows the scheme for administering Talabostat mesylate and Pembrolizumab to subjects with mCrPC during the treatment Lead-in Stage and the Efficacy Stage according to Example 1.

FIG. 2: shows the prostate specific antigen (PSA) levels in ten subjects according to Example 1.

FIG. 3: shows the scheme for administering Talabostat mesylate and Pembrolizumab to subjects with advanced solid cancers during the treatment Lead-in Stage according to Example 2.

FIG. 4: shows the Phase 1b/2 scheme according to Example 1.

FIG. 5: shows the plasma concentration of individual subjects over multiple dosing cycles after 0.4 mg or 0.6 mg total daily dose of Talabostat mesylate [individual plots] according to Example 1.

FIG. 6: shows the plasma concentration profiles in 3 cycles after 0.4 mg or 0.6 mg according to Example 1

FIG. 7: shows the IL-18 changes in the serum post dosing of 0.4 mg QD (Talabostat mesylate)+Pembrolizumab (n=3) according to Example 1.

FIG. 8: shows the IL-18 changes in the serum post dosing of 0.6 mg QD (Talabostat mesylate)+Pembrolizumab (n=1) according to Example 1.

FIG. 9: shows the IL-18 changes in the serum post dosing of 0.6 mg split dose (0.3 mg BID-Talabostat mesylate)+Pembrolizumab (n=4) according to Example 1.

FIG. 10: shows the IFN-γ changes in the serum post dosing of 0.4 mg QD (Talabostat mesylate)+Pembrolizumab (n=3) according to Example 1.

FIG. 11: shows the IFN-γ changes in the serum post dosing of 0.6 mg QD (Talabostat mesylate)+Pembrolizumab (n=1) according to Example 1.

FIG. 12: shows the IFN-γ changes in the serum post dosing of 0.6 mg split dose (0.3 mg BID-Talabostat mesylate)+Pembrolizumab (n=4) according to Example 1.

FIG. 13: shows the IL-1β changes in the serum post dosing of 0.4 mg QD (Talabostat mesylate)+Pembrolizumab (n=3) according to Example 1.

FIG. 14: shows the IL-1β changes in the serum post dosing of 0.6 mg QD (Talabostat mesylate)+Pembrolizumab (n=1) according to Example 1.

FIG. 15: shows the IL-1β changes in the serum post dosing of 0.6 mg split dose (0.3 mg BID-Talabostat mesylate)+Pembrolizumab (n=4) according to Example 1.

FIG. 16: shows the IL-6 changes in the serum post dosing of 0.6 mg split dose (0.3 mg BID-Talabostat mesylate)+Pembrolizumab (n=4) according to Example 1

FIG. 17: shows the IL-8 changes in the serum post dosing of 0.6 mg split dose (0.3 mg BID-Talabostat mesylate)+Pembrolizumab (n=4) according to Example 1.

FIG. 18: shows best change in sum of target lesions per RECIST 1.1 according to Example 2

FIG. 19A: shows partial response (−64%) in a patient with MSS, PDL-1 negative endometrial cancer and 2 prior systemic therapies not including PD-1/PD-L1 or CTLA4 inhibitors according to Example 2. The patient continues on study for more than 4 months.

FIG. 19B: shows stable disease (−23%) in a patient with high grade pleomorphic sarcoma, MSS, PDL-1 negative and 3 prior systematic therapies including PD-1 antibody (best response: PD) to Example 2. The patient continues on study for more than 5 months.

FIG. 20: shows serum PSA % change from baseline over time in Phase 1b and Phase 2 patients according to Example 1.

FIG. 21: shows PSA Best Overall response in Phase 1b and Phase 2 patients according to Example 1.

### DETAILED DESCRIPTION

In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Abbreviations

As used herein, the following abbreviations have the following meanings:

A2AR: A2A adenosine receptor

ADT: Androgen deprivation therapy

ALK: Anaplastic lymphoma kinase

ALT: Alanine aminotransferase

ANC: Absolute neutrophil count

AR: Androgen receptor

AST: Aspartate aminotransferase

AUC: Area under the plasma concentration-time curve

AUC 0-last: Area under the plasma concentration time curve for the last measurable concentration

BS: Bone scintigraphy

BUN: Blood urea nitrogen

CAF: Cancer associated fibroblast

CLL: Chronic lymphocytic leukemia

CR: Complete response

CRPC: Castration-resistant prostate cancer

CT: Computed tomography

CTC: Circulating tumor cells

ctDNA: Circulating tumor DNA

CTLA4: Cytotoxic T-lymphocyte associated protein 4

CPS: Combined positive scores

DPP: Dipeptidyl peptidase

DKA: Diabetic ketoacidosis

DLT: Dose limiting toxicity

DOR: Duration of response

DSRC: Data Safety Review Committee

EGFR: Epidermal growth factor receptor

ECOG: Eastern Cooperative Oncology Group

eCRF: Electronic case report form

EOT: End of Treatment

FAP: Fibroblast activation protein

GM-CSF: Granulocyte-macrophage colony-stimulating factor

G-CSF: Granulocyte-colony stimulating factor

GCP: Good Clinical Practice

HER2: Human epidermal growth factor receptor 2

HCC: hepatocellular carcinoma

ICI: Immune check point inhibitor

IC50: Half maximal inhibitory concentration

ICH: International Council for Harmonisation

IEC: Independent Ethics Committee

IL: Interleukin

IDO: Indoleamine 2,3-dioxygenase

IMT: Inflammatory myofibroblastic tumor

irCR: Immune-related complete disease

irPR: Immune-related partial response

irSD: Immune-related stable disease

IND: Investigational New Drug (Application)

IRB: Institutional Review Board

iRECIST: Immune Response Evaluation Criteria In Solid tumors

ITT: Intent-to-Treat

LAG3: Lymphocyte activation gene 3 protein

LDH: Lactate dehydrogenase

LHRH: Luteinizing hormone-releasing hormone

mCrPC: metastatic castration resistant prostate cancer

MSI-H: Microsatellite instability-high

MDSC: Myeloid derived suppressor cell

MedDRA: Medical Dictionary for Regulatory Activities

MRI: Magnetic resonance imaging

mRNA: Messenger ribonucleic acid

NK: Natural killer

NCI CTCAE: National Cancer Institute Common Terminology Criteria for Adverse Events

NEPC: Neuroendocrine prostate cancer

NHL: Non-Hodgkin's lymphoma

NSCLC: Non-small cell lung cancer

OS: Overall survival

PCWG3: Prostate Cancer Working Group 3

PD: Progressive disease

PD-1: Programmed cell death 1

PD L1: Programmed death ligand 1

PD L2: Programmed death ligand 2

PFS: Progression-free survival

PR: Partial response

PD-1: Programmed Cell Death 1

Q.D: Quaque die

QTcB: QT interval corrected for heart rate using Bazett's formula

RECIST: Response Evaluation Criteria In Solid Tumors

rPFS: Radiographic progression-free survival

SD: Stable disease

SAE: Serious adverse event

SAP: Statistical Analysis Plan

SJS: Stevens-Johnson syndrome

SCNC: Small cell neuroendocrine prostate cancer

sHASEGP: Soluble neutral-active hyaluronidase glycoproteins

TIM3: T-cell immunoglobulin and mucin-domain containing-3

Treg: Regulatory T cells or T-regulatory cells

t NEPC: treatment emergent neuroendocrine prostate cancer

TPS: Tumor Proportion Score

TRAE: Treatment related adverse events

TEN: Toxic epidermal necrolysis

T1DM: Type 1 diabetes mellitus

Tmax: Time of maximum observed concentration

ULN: Upper limit of normal

The therapeutic agents Talabostat and Pembrolizumab as intended for use in the present disclosure are described below:

### Therapeutic Agents

Talabostat or a pharmaceutically acceptable salt thereof: Talabostat is referred to interchangeably as PT-100, Talabostat (USAN), and [(2R)-1-[(2S)-2-amino-3-methyl-1-oxobutyl]-2-pyrrolidinyl] boronic acid. Talabostat has a CAS registration number of 149682-77-9. Talabostat, also known as Val-boro-pro (L-valinyl-L-boroproline), is disclosed in PCT Appl. Publication No. 1989/003223. The IUPAC name of talabostat is [(2R)-1-[(2S)-2-amino-3-methylbutanoyl]pyrrolidin-2-yl]boronic acid. Talabostat (PubChem ID: 6918572), or a pharmaceutically acceptable salt thereof, such as, for example, talabostat mesylate (PubChem CID: 11522448). In some aspects, the free base may be used. In other aspects, the Talabostat or a pharmaceutically acceptable salt thereof may be a solvate. In most clinical formulations, Talabostat is provided as a salt form, e.g. Talabostat mesylate. Talabostat has two chiral centers with a R, S configuration. Talabostat or a pharmaceutically acceptable salt thereof can exist as both linear and cyclic forms (R J Snow et al., J. Am. Chem. Soc., 1994, 116 (24), pp 10860-10869).

Talabostat or a pharmaceutically acceptable salt thereof is effective for the treatment of cancer by modulating multiple intracellular and extracellular dipeptidyl peptidases. More specifically, intracellular and extracellular dipeptidyl peptidases comprise Fibroblast Activation Protein, DPP 8/9, CD26/DPP4 and DPP2. Talabostat or a pharmaceutically acceptable salt thereof has a dual mechanism of action which includes stromal targeted activity via FAP inhibition and targeted immunostimulatory activity via DPP 8/9 inhibition. Talabostat inhibits FAP enzymatic activity thereby suppressing tumor growth. Talabostat or a pharmaceutically acceptable salt thereof also inhibits DPP8/9 thereby inducing an IL 113 response (via caspase-1) in the stroma of tumor and lymph nodes. Talabostat's dual mechanism of action introduces a novel approach to the treatment of cancer because it combines both tumor-targeted and immune-stimulatory activity in a single agent.

Pembrolizumab: Pembrolizumab (also known as MK-3475, Lambrolizumab, KEYTRUDA®, and SCH-900475) is a humanized antibody, which targets the PD-1 receptor of lymphocytes, thereby blocking PD-1 inhibitory signal transduction. Pembrolizumab may be readily procured from the marketplace.

### Methods of Use:

The present disclosure is based, in part, on an improved regimen to treat prostate cancer (e.g. SCNC, NEPC, tNEPC, CrPC, mCrPC, adenocarcinoma) comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle. The administration of Talabostat or a pharmaceutically acceptable salt thereof and Pembrolizumab may produce an overall enhanced anti-cancer effect, such as improved T-cell priming, increased T cell stimulation, increased infiltration of neutrophil and macrophages across tumor microenvironment, decreased tumor volume, increased activation of natural killer cells, enhanced activation of dendritic cells, synergistic increase in pro-inflammatory cytokine (ILL IL2, IL18, IFN gamma, IL6, IL12p40, IL 15, IL 7, G-CSF and GM-CSF), enhanced anti-tumor memory response, reduced metastasis and reduced toxicity.

In some embodiments, the present disclosure provides a method of treating a subject afflicted with one or more solid tumors (e.g. an advanced solid tumor), comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle. In another embodiment, the method comprises one or more treatment cycles, wherein each cycle of about 21 days duration.

An advantage of using the particular regimen of Talabostat or a pharmaceutically acceptable salt thereof and Pembrolizumab of this disclosure is in the curtailment of the progression of prostate cancer (e.g. SCNC, NEPC, tNEPC, CrPC, mCrPC, adenocarcinoma) reduction in tumor burden, reduced metastasis and/or inducement of tumor regression in a subject.

In some embodiments, the present disclosure provides a method of delaying progression of or preventing or delaying tumor recurrence, tumor growth or spread of tumor in a subject afflicted with prostate cancer (e.g. SCNC, NEPC, tNEPC, CrPC, mCrPC, adenocarcinoma), comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle.

In some embodiments, the present disclosure provides a method of delaying or preventing the progression of tumor recurrence, tumor growth or spread of a tumor in a subject afflicted with one or more solid tumors (e.g. an advanced solid tumor), comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle.

In some embodiments, provided herein is a method for initiating, sustaining or enhancing an anti-tumor immune response in a subject afflicted with prostate cancer (e.g. SCNC, NEPC, tNEPC, CrPC, mCrPC, adenocarcinoma), the method comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle. In another aspect, provided herein is a method for initiating, sustaining or enhancing an anti-tumor immune response in a subject afflicted with one or more solid tumors (e.g. an advanced solid tumor), comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle.

In some embodiments, the treatment regimen described herein results in a sustained response in the subject after cessation of the treatment.

In some embodiments, the subject has a prostate cancer that may be at an early stage or a late stage. In some embodiments, the prostate cancer is metastatic.

In some embodiments, the cancer is an advanced malignant solid neoplasm or cancer. In some embodiments, the cancer is recurrent malignant solid neoplasm. In some embodiments, the subject is a human.

In some embodiments, the solid cancer is selected from the group comprising of castrate resistant prostate cancer, endometrial cancer, dedifferentiated liposarcoma, basal cell carcinoma, leiomyosarcoma, squamous cell carcinoma of unknown primary, mucosal melanoma, skin melanoma, pleomorphic sarcoma, colorectal/colon cancer, astrocytoma, triple negative breast cancer, uterine sarcoma, uveal melanoma.

In some embodiments, the patient is pretreated with PD-1/PD-L1 or CTLA-4 antibodies. In some embodiments, the patient is PD-1/PD-L1 or CTLA-4 naïve. In some embodiments, the subject has relapsed or progressed with PD-1/PD-L1 or CTLA-4 antibodies.

In some embodiments, the present disclosure provides a method for reducing the treatment related adverse effects (TRAEs) in a subject afflicted with prostate cancer (e.g. SCNC, NEPC, tNEPC, CrPC, mCrPC, adenocarcinoma), comprising administering Talabostat or a pharmaceutically acceptable salt thereof and Pembrolizumab in the treatment regimen described herein. It was surprisingly discovered subjects administered a split dose of 0.3 mg of Talabostat twice daily experienced increased tolerability and significantly less treatment-related adverse events (TRAEs) relative to a subject with the same cancer that was administered a single 0.6 mg daily dose of Talabostat.

TRAEs contemplated within the scope of this invention are readily apparent to a person of ordinary skill in the art, and include, but are not limited to hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, myalgia, constipation, hypocalcemia, neutropenia, febrile neutropenia, anemia, leukopenia, pancytopenia, lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest. In some embodiments, the subject experiences no TRAEs.

In some embodiments, a subject with one or more solid tumors (e.g. an advanced solid tumor) experiences less TRAEs relative to a subject with the same cancer administered a single 0.6 mg daily dose of Talabostat. In some embodiments, the subject experiences no TRAEs.

In some embodiments, the treatment regimen of the present disclosure produces an increased innate immune response as compared to the innate immune response when the subject is administered Talabostat alone. The innate immune response may be increased by infiltration of innate immune cells, in particular macrophages into the blood, and NK-cells into the tumor. Further, the present treatment regimen comprising Talabostat or a pharmaceutically acceptable salt thereof and Pembrolizumab can produce suppression of the Treg function that is greater than that obtained using Talabostat alone.

In some embodiments, the prostate cancer is selected from small cell neuroendocrine prostate cancer (SCNC), neuroendocrine prostate cancer (NEPC), treatment emergent neuroendocrine prostate cancer (tNEPC), castration resistant prostate cancer (CrPC), adenocarcinoma type prostate cancer. In another embodiment, the solid cancer is selected from the group comprising castrate resistant prostate cancer, endometrial cancer, dedifferentiated liposarcoma, basal cell carcinoma, leiomyosarcoma, squamous cell carcinoma of unknown primary, mucosal melanoma, skin melanoma, pleomorphic sarcoma, colorectal/colon cancer, astrocytoma, triple negative breast cancer, uterine sarcoma, uveal melanoma.

The present treatment regimen comprising Talabostat or a pharmaceutically acceptable salt thereof and Pembrolizumab can also significantly increase the tumor infiltration of immune sub-populations, such as NK-cells and macrophages, compared to monotherapies using Talabostat or a pharmaceutically acceptable salt thereof and Pembrolizumab.

### Treatment Regimens:

In some embodiments, each treatment cycle is of 21-days duration and Talabostat or a pharmaceutically acceptable salt thereof is administered on each of days 1 to 14 and Pembrolizumab is administered on day 1. Said regimen is effective to treat a subject afflicted with various forms of prostate cancer (e.g. SCNC, NEPC, tNEPC, CrPC, mCrPC, adenocarcinoma). The regimen herein disclosed for treating prostate cancer (e.g. SCNC, NEPC, tNEPC, CrPC, mCrPC, adenocarcinoma) may also be used more generally to treat a subject with a solid tumor (e.g. an advanced solid tumor).

Pembrolizumab may conveniently be administered as a single dose in the regimen of this disclosure to effectively treat a subject afflicted with prostate cancer (e.g. SCNC, NEPC, tNEPC, CrPC, mCrPC, adenocarcinoma) or a subject afflicted with a solid tumor, e.g. an advanced solid tumor.

In some embodiments, Pembrolizumab may be administered at a total dose of about 1 mg/kg to about 10 mg/kg, conveniently by injection (e.g., intravenously), most preferably as continuous infusion for 30 minutes. In some embodiments, a suitable dose of Pembrolizumab administered intravenously in the treatment regimen of the present disclosure may conveniently be from about 100 mg to about 500 mg, e.g. about 200 mg. In some embodiments, Pembrolizumab is administered intravenously at a total dose of about 200 mg on day 1 of each treatment regimen.

In some embodiments, Pembrolizumab (MK-3475) is administered as a liquid medicament which comprises 25 mg/ml MK-3475, 7% (w/v) sucrose, 0.02% (w/v) polysorbate 80 in 10 mM histidine buffer pH 5.5, and the selected dose of the medicament is administered by IV infusion over a time period of about 30 minutes.

In some embodiments, Talabostat or a pharmaceutically acceptable salt thereof is administered twice a day. In some embodiments, Talabostat or a pharmaceutically acceptable salt thereof is administered at a dose of 0.3 mg twice a day. In some embodiments, Talabostat or a pharmaceutically acceptable salt thereof is administered at a dose 0.2 mg twice a day for a sufficient time (e.g. for 1 to 30 days) followed by 0.3 mg twice a day thereafter. For example, Talabostat or a pharmaceutically acceptable salt thereof is administered at a dose 0.2 mg twice a day for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, followed by 0.3 mg twice a day thereafter (i.e. for the remainder of the treatment cycle and for each treatment cycle thereafter). In some embodiments, Talabostat or a pharmaceutically acceptable salt thereof is administered at a dose of about 0.2 mg twice daily on days 1-7 of the first treatment cycle followed by about 0.3 mg twice daily on days 8-14 of the first treatment cycle. In some embodiments, Talabostat or a pharmaceutically acceptable salt thereof may be administered at 0.3 mg as a morning dose (e.g. about 5 am, about 6 am, about 7 am, about 8 am, about 9 am, about 10 am, or about 11 am) and 0.3 mg as an evening dose (e.g. about 5 pm, about 6 pm, about 7 pm, about 8 pm, about 9 pm, about 10 μm, or about 11 pm).

In some embodiments, each treatment cycle is from 1 to 30 days in duration. For example, each treatment cycle is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, each treatment cycle is 21 days in duration. In some embodiments, the subject is administered Talabostat or a pharmaceutically acceptable salt thereof and Pembrolizumab for one or more treatment cycles. For example, in some embodiments, the subject is administered Talabostat or a pharmaceutically acceptable salt thereof and Pembrolizumab for 1, 2, 3, 4, 5, 6, 7, 8, 9, 10, 11, 12, 13, 14, 15, 16, 17, 18, 19, or 20 treatment cycles. In some embodiments, the subject is administered Talabostat or a pharmaceutically acceptable salt thereof and Pembrolizumab for more than one treatment cycle.

In some embodiments, the Talabostat and Pembrolizumab are administered according to an intermittent dosing regimen. The term “intermittent dosing regimen” refers to repeating cycles of drug administration in which the drug is administered on one or more consecutive days (“days on”) followed by one or more consecutive days of rest on which the drug is not administered (“days off”). The cycles may be regular, in that the pattern of days on and days off is the same in each cycle, or may be irregular. In some embodiments, the cycle is 21 days and Talabostat or a pharmaceutically acceptable salt thereof is administered for 1, 2, 3, 4, 5, 6, 7, 8, 9, 10, 11, 12, 13, 14, 15, 16, 17, 18, 19, or 20 consecutive days followed by 1, 2, 3, 4, 5, 6, 7, 8, 9, 10, 11, 12, 13, 14, 15, 16, 17, 18, 19, or 20 days where the patient takes no Talabostat. In some embodiments, this intermittent dosing schedule occurs multiple times within the same cycle. For example, in some embodiments, the cycle is 21 days and Pembrolizumab is administered intravenously on day 1 and Talabostat or a pharmaceutically acceptable salt thereof is administered for 3 consecutive days followed by 4 days where the patient takes no Talabostat, and this 7 day pattern repeats once (i.e. no Talabostat is administered on days 15-21) or twice. In some embodiments, Talabostat or a pharmaceutically acceptable salt thereof is administered orally (e.g. by tablet) on each of days 1 to 14 and Pembrolizumab is administered intravenously on day 1 and no drug is administered on days 15-21.

In some embodiments, the subject experiences an increase in pro-inflammatory cytokines relative to a subject that is administered a single 0.6 mg daily dose of Talabostat. Pro-inflammatory cytokines contemplated within the scope of this invention are readily apparent to a person of ordinary skill in the art, and include, but are not limited to IL-6, IL-8, IL-18, IFN-γ, and IL-1β. In some embodiments, the pro-inflammatory cytokines are one or more of IL-18 and IFN-γ. In some embodiments, wherein the maximum increase in cytokines is observed at day 14 of continuous dosing.

Suitable treatment regimens for treating a human patient afflicted with prostate cancer (e.g. SCNC, NEPC, tNEPC, CrPC, mCrPC, adenocarcinoma) include, for example, administering to the patient Talabostat or a pharmaceutically acceptable salt thereof at a dose of 0.3 mg twice daily and effective amount of Pembrolizumab. In some embodiments, the regimen comprises one or more administration cycles (e.g. 1, 2, 3, 4, 5, 6 or more cycles). In some embodiments, each cycle is a period of about 21 days. In some embodiments, Talabostat or a pharmaceutically acceptable salt thereof is administered orally (e.g. by tablet) on each of days 1 to 14 and Pembrolizumab is administered intravenously on day 1 where no drug is administered on days 15-21. The same regimen treatment may be used generally to treat a human patient afflicted with one or more solid tumors (e.g. an advanced solid tumor).

In some embodiments, during one or more treatment cycles of about 21 days, Talabostat is administered on days 1 to 14 at a total daily dose of about 0.4 mg to about 0.6 mg and Pembrolizumab is administered on day 1 at a total dose of about 100 mg to about 500 mg per day, e.g. about 200 mg per day.

In some embodiments, the daily dose of Talabostat or a pharmaceutically acceptable salt thereof may be varied over time. For example, during the first cycle (including the Lead-in Stage) Talabostat or a pharmaceutically acceptable salt thereof may be administered at a lower daily dose than during subsequent cycles (e.g. Efficacy Stage). For example, Talabostat or a pharmaceutically acceptable salt thereof may conveniently be administered during the Lead-in Stage at a daily dose of about 0.4 mg, and, if there are no side effects or other criteria preventing further treatment, the subject is allowed to enter the Efficacy Stage and administered in divided doses, during Efficacy Stage, a total daily dose of about 0.6 mg. In some embodiments, the Lead-in Stage and the Efficacy Stage occur in the same cycle (e.g. the dose of Talabostat in the Lead in Stage is 0.2 mg twice a day on days 1-7, followed a dose of 0.3 mg twice a day thereafter during the Efficacy Stage). In another embodiment, Talabostat or a pharmaceutically acceptable salt thereof may be administered in divided doses during the Lead-in Stage at total daily dose of about 0.6 mg and during the Efficacy Stage a daily dose of about 0.4 mg. In another embodiment, the patient is treated directly in the Efficacy Stage using a daily dose of about 0.4 mg or about 0.6 mg. In another embodiment, the patient is treated directly in the Efficacy Stage using a total daily dose of about 0.6 mg. In another embodiment, the patient is treated directly in the Efficacy Stage using a total daily dose of about 0.6 mg administered as a split dose 0.3 mg twice a day.

In some embodiments, Talabostat or a pharmaceutically acceptable salt thereof and Pembrolizumab are administered to a subject afflicted with prostate cancer (e.g. SCNC, NEPC, tNEPC, CrPC, mCrPC) in any desired number of treatment cycles as long as a clinical benefit is observed or until there is a complete response, confirmed progressive disease or unmanageable toxicity. In some embodiments, the treatment regimen is administered to a subject afflicted with one or more solid tumors (e.g. an advanced solid tumor) in any desired number of treatment cycles as long as a clinical benefit is observed or until there is a complete response, confirmed progressive disease or unmanageable toxicity.

In some embodiments, the daily dosages of Talabostat or a pharmaceutically acceptable salt thereof and Pembrolizumab used according to the treatment regimen of this disclosure may be lower than the daily dosages of one or both of Talabostat or a pharmaceutically acceptable salt thereof and Pembrolizumab administered as single agents to treat a subject afflicted with prostate cancer (e.g. SCNC, NEPC, tNEPC, CrPC, mCrPC, adenocarcinoma). In some embodiments, the subject is afflicted with one or more solid tumors (e.g. an advanced solid tumor).

In some embodiments, the combination therapy comprising 0.3 mg twice daily dose of Talabostat or a pharmaceutically acceptable salt thereof and 200 mg of pembrolizumab is administered for at least 12 weeks (three 4-week cycles or four 3-week cycles) or at least 24 weeks six 4-week cycles or eight 3-week cycles). In some embodiments, the combination therapy is administered for at least 1-4 weeks after the patient achieves a complete response.

In some embodiments, a single administration cycle comprises 21 days (a 21-day cycle). In some embodiments, Talabostat mesylate is administered once daily (QD) on Days 1 to 14 of a 21-day cycle plus pembrolizumab 200 mg administered intravenously (IV) on Day 1 every 21 days. In some embodiments, neither Talabostat nor Pembrolizumab are delivered on days 15-21.

In some embodiments, a single administration cycle comprises 21 days (21-day cycle). In specific embodiment, Talabostat mesylate is administered twice daily at a dose of 0.3 mg on Days 1 to 14 of a 21-day cycle plus pembrolizumab 200 mg administered intravenously (IV) on Day 1 every 21 days. In another embodiment, Talabostat mesylate is administered at a dose of about 0.3 mg in the morning and about 0.3 mg in the evening on Days 1 to 14 of a 21-day cycle plus pembrolizumab 200 mg administered intravenously (IV) on Day 1 every 21 days. In another embodiment, Talabostat mesylate is administered thrice daily at a dose of about 0.2 mg on Days 1 to 14 of a 21-day cycle plus pembrolizumab 200 mg administered intravenously (IV) on Day 1 every 21 days. In another embodiment, Talabostat mesylate is administered at a dose of about 0.4 mg in the morning and about 0.2 mg in the evening on Days 1 to 14 of a 21-day cycle plus pembrolizumab 200 mg administered intravenously (IV) on Day 1 every 21 days. In another embodiment, Talabostat mesylate is administered at a dose of about 0.2 mg in the morning and about 0.4 mg in the evening on Days 1 to 14 of a 21-day cycle plus pembrolizumab 200 mg administered intravenously (IV) on Day 1 every 21 days. In another embodiment, Talabostat mesylate is administered twice daily at a dose of about 0.2 mg on Days 1 to 7 of a 21-day cycle plus pembrolizumab 200 mg administered intravenously (IV) on Day 1 every 21 days followed by Talabostat mesylate is administered twice daily at a dose of about 0.3 mg on Days 8 to 14 of a 21-day cycle. In some embodiments, Talabostat mesylate is administered twice daily at a dose of about 0.3 mg on Days 1 to 3 of a 21-day cycle plus pembrolizumab 200 mg administered intravenously (IV) on Day 1 every 21 days followed by rest period from day 4-7 (no dose of Talabostat given) and then followed by Talabostat mesylate is administered twice daily at a dose of about 0.3 mg on Days 8 to 11 of a 21-day cycle

In some embodiments, a combination therapy of the disclosure is administered to a patient who has not been previously treated with a biotherapeutic or chemotherapeutic agent (i.e. is treatment-naïve). In other embodiments, the combination therapy is administered to a patient who failed to achieve a sustained response after prior therapy with a biotherapeutic or chemotherapeutic agent (i.e. is treatment-experienced).

In some embodiments, the subject was treated with a single daily 0.6 mg dose of Talabostat mesylate and experienced dose-limiting side effects.

A suitable time period of therapy can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. For example a suitable time period of therapy can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive.

### Pharmaceutical Formulations

In some embodiments, the present disclosure provides for use in the treatment regimen herein a pharmaceutical formulation comprising an effective amount of Talabostat or a pharmaceutically acceptable salt thereof and an effective amount of Pembrolizumab together with one or more pharmaceutically acceptable carriers or adjuvants. Any of the pharmaceutically acceptable carriers or adjuvants described herein, or known in the art, may be used. As used herein, the term “pharmaceutical formulation” refers to a formulation comprising Talabostat or a pharmaceutically acceptable salt thereof or a formulation comprising Pembrolizumab, wherein each formulation also comprises one or more pharmaceutically acceptable carriers or adjuvants. Pharmaceutically acceptable carriers or adjuvants are well-known to those skilled in the art, and usually depend on the chosen route of administration.

In some embodiments, a first formulation comprising Talabostat or a pharmaceutically acceptable salt thereof and one or more pharmaceutically acceptable carriers or adjuvants and a second formulation comprising Pembrolizumab and one or more pharmaceutically acceptable carriers or adjuvants are administered according to the treatment regimen disclosed herein to produce a synergistic effect in treating a subject afflicted with prostate cancer (e.g. SCNC, NEPC, tNEPC, CrPC, adenocarcinoma). In some embodiments, a first formulation comprising Talabostat or a pharmaceutically acceptable salt thereof and one or more pharmaceutically acceptable carriers or adjuvants and a second formulation comprising Pembrolizumab and one or more pharmaceutically acceptable carriers or adjuvants are administered according to the treatment regimen disclosed herein to produce a synergistic effect in treating a subject afflicted with one or more solid tumors e.g. an advanced solid tumor.

The pharmaceutical formulations may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions may be formulated as the injectable or infusible solutions. The formulation is in a form suitable for oral, intravenous, intraarterial, intramuscular, subcutaneous, parenteral, transmucosal, transdermal, or topical administration. The formulation may be formulated as an immediate, controlled, extended or delayed release composition.

In some embodiments, the formulation comprising Talabostat or a pharmaceutically acceptable salt thereof may be administered orally. In some embodiments, the formulation comprising Pembrolizumab may be administered parenterally. As used herein, the term “parenteral” includes subcutaneous, intravenous, intra-arterial, intraperitoneal, intracardiac, intrathecal, and intramuscular injection, as well as infusion injections.

Liquid pharmaceutical formulations for parenteral administration may be formulated for administration by injection or continuous infusion. In some embodiments, parenteral formulations can include prefilled syringes, vials, powder for infusion for reconstitution, concentrate for infusion to be diluted before delivery (ready to dilute), solutions (ready to use).

Injectable pharmaceutical formulations can be aqueous isotonic solutions or suspensions, and suppositories can be prepared from fatty emulsions or suspensions.

Pharmaceutical formulations formulated for parenteral administration (e.g. via intravenous injection) may conveniently include a liquid carrier, or may be reconstituted into a liquid solution or suspension for parenteral administration.

In general, such formulations typically comprise a pharmaceutically acceptable carrier or adjuvant. As used herein, the term “pharmaceutically acceptable” means approved by a government regulatory agency or listed in the U.S. Pharmacopoeia or another generally recognized pharmacopoeia for use in animals, particularly in humans.

Pharmaceutical formulations of Pembrolizumab for intravenous administration may be purchased from the marketplace or prepared using conventional formulation techniques. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride, hexamethonium chloride, benzalkonium chloride, benzethonium chloride, phenol, butyl or benzyl alcohol, chlorobutanol, thimerosal, alkyl parabens such as methyl or propyl paraben, catechol, resorcinol, cyclohexanol, 3-pentanol and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; chelating agents such as EDTA; monosaccharides, disaccharides, and other carbohydrates including sugars such as sucrose, mannitol, trehalose or sorbitol, glucose, mannose, or dextrins; salt-forming counter-ions such as sodium; metal complexes (for example, Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). The carrier can be a solvent or reconstitution medium or dispersion medium containing, for example, water, ethanol, polyol (for example, glycerol, propylene glycol, and liquid polyethylene glycol, and the like), and suitable mixtures thereof. More particularly, the pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, liquid polyethylene glycol, or the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the formulation includes isotonic agents, for example, sugars, polyalcohols, such as mannitol, sorbitol, or sodium chloride. For intravenous administration, suitable carriers include physiological saline, bacteriostatic water, Cremophor EL (BASF, Parsippany, N.J.) or phosphate buffered saline (PBS). Prolonged absorption of the injectable compositions can be brought about by including in the composition an agent which delays absorption, for example, aluminium monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will preferably have labels or package inserts indicating that the associated compositions are useful for treating a subject suffering from or predisposed to autoimmune or neoplastic disorders. The pharmaceutical formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

Solution or suspension formulations used for subcutaneous application typically include one or more of the following components: a sterile carrier such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical formulations of Talabostat or a pharmaceutically acceptable salt thereof for oral use herein may be administered, for example, in the form of tablets, capsules, powders, dispersible granules, sachets etc., or as aqueous solutions or suspensions, preferably tablets. Oral compositions generally include an inert carrier (for example, diluent) or an edible carrier. The formulations may be enclosed in a gelatin capsule or compressed into a tablet. Pharmaceutically compatible binding agents, and/or adjuvant materials can be included as part of the formulation. The tablets, pills, capsules, granules, sachets, troches and the like can contain any of the following ingredients, or compounds of a similar nature; a binder such as microcrystalline cellulose, gum tragacanth or gelatin; an excipient such as starch or lactose, a disintegrating agent such as alginic acid, primogel, or corn starch; a lubricant such as magnesium stearate or stearates; a glidant such as colloidal silicon dioxide; a sweetening agent such as sucrose or saccharin; or a flavouring agent such as peppermint, methyl salicylate, or orange flavouring.

In some embodiments, an oral pharmaceutical formulation comprising Talabostat or a pharmaceutically acceptable salt thereof described herein may comprise one or more pharmaceutically acceptable carriers or adjuvants selected from the group comprising a bulking agent, buffer, surfactant, and pH modifier. The pharmaceutical formulation may be adjusted to give an appropriate pH.

In a particular embodiment, Talabostat or a pharmaceutically acceptable salt thereof is formulated as a tablet for oral administration according to the treatment regimen of this disclosure. The pharmaceutical tablet may be an immediate release or a modified release tablet. Tablet may be in the form of matrix or coated form.

In certain embodiments, the various processes of making above mentioned formulations or compositions are included and such formulations can be manufactured by any of the processes known in the art.

An exemplary immediate release tablet comprises an effective amount of Talabostat or a pharmaceutically acceptable salt thereof and one or more pharmaceutically acceptable carriers selected from diluents, binders, disintegrants, glidants, lubricants, pH modifying agents and combinations thereof.

Diluents: one or more diluents comprise, but are not limited to dibasic calcium phosphate, pullulan, maltodextrin, isomalt, sugar pellets, mannitol, spray-dried mannitol, microcrystalline cellulose, dibasic calcium phosphate dihydrate, lactose, sugars, sorbitol, mixture of microcrystalline cellulose and guar gum (Avicel CE-15), mixture of mannitol, polyplasdone and syloid (Pharmaburst), mixture of mannitol, crospovidone and polyvinyl acetate (Ludiflash), isomalt, Panexcea, F-Melt, sucrose, calcium salts and similar inorganic salts, heavy magnesium carbonate and the like, and the mixtures thereof. Preferably, it is lactose or microcrystalline cellulose.

Binders: one or more binders comprise, but are not limited to, low-substituted hydroxypropyl cellulose, xanthan gum, polyvinylpyrrolidone (povidone), gelatin, sugars, glucose, natural gums, gums, synthetic celluloses, polymethacrylate, hydroxypropyl methylcellulose, hydroxypropyl cellulose, carboxymethyl cellulose, methyl cellulose, and other cellulose derivatives and the like, and the mixtures thereof. Preferably, the binder is polyvinylpyrrolidone or hydroxypropyl cellulose or hydroxypropyl methylcellulose.

Disintegrants: one or more binders comprise, but are not limited to, at least one or a mixture of sodium starch glycolate, croscarmellose sodium, crospovidone, sodium alginate, gums, starch, and magnesium aluminium silicate. Preferably, the disintegrant is sodium starch glycolate.

Lubricants: one or lubricants comprise, but are not limited to sodium stearyl fumarate, sodium lauryl sulphate, magnesium stearate, polyethylene glycol, metal stearates, hydrogenated castor oil and the like, and the mixtures thereof. Preferably, the lubricant is magnesium stearate.

Glidant: one or glidants comprise, but are not limited to, stearic acid, colloidal silicon dioxide, talc, aluminium silicate and the like, and the mixtures thereof. Preferably, it is talc.

pH modifying agents: one or more pH modifying agents comprise, but are not limited to organic acid or its salts like phosphoric acid, citric acid and the like.

In one embodiment, the relative percentages of the ingredients in tablet formulations of Talabostat is given below in Table 1:

TABLE 1

| Formulation Content | Amount (w/w %) |
|---|---|
| Talabostat as an API (available as | 0.01-2 |
| Talabostat mesylate) | |
| Binder | 1-50 |
| Disintegrant | 1-15 |
| Lubricant | 0.1-5 |
| Diluent | 30-98 |
| pH modifying agent | 0-15 |

An exemplary immediate release tablet of Talabostat mesylate is given below in Table 2:

TABLE 2

| | Preferred | Amount |
|---|---|---|
| Formulation content | ranges (w/w %) | (w/w %) |
| Talabostat mesylate | 0.01-2 | 0.145 |
| (69% free base) | | |
| Polyvinylpyrrolidone or | 1-50 | 1.00 |
| hydroxypropylcellulose or | | |
| hydroxypropylmethylcellulose | | |
| or pregelatinized starch as a | | |
| binder | | |
| Sodium starch glycolate or | 1-15 | 2.5 |
| crospovidone as a disintegrant | | |
| Stearic acid as a lubricant | 0.1-5 | 1.5 |
| Lactose as a diluent | 30-90 | 85.315 |
| Microcrystalline cellulose as a | 5-20 | 9.480 |
| diluent | | |
| Sodium phosphate monobasic, | 0-15 | 0.060 |
| monohydrate as a pH | | |
| modifying agent | | |
| Phosphoric acid as a pH | For pH adjustment | For pH adjustment |
| modifying agent | | |

In some embodiments, Talabostat or a pharmaceutically acceptable salt thereof may be formulated as a modified release matrix tablet. An exemplary extended release tablet comprises an effective amount of Talabostat or a pharmaceutically acceptable salt thereof and pharmaceutically-acceptable carriers or adjuvants are selected from diluents, binders, modified release material, glidants, lubricants, colorants and combinations thereof. Alternatively, a modified release tablet comprises immediate release core and coating wherein said coating comprises modified release material and other pharmaceutical excipients.

Modified release materials comprise, but are not limited to: polyvinylpyrrolidone (K90), Hydroxypropylmethylcellulose (K4M, K10), hydroxypropylcellulose (high viscosity grade), carnauba wax, glyceryl behenate, castor wax, polyvinyl acetate, carboxymethyl ethyl cellulose, ethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide and the like. Particular modified release materials include polyvinylpyrrolidone (K90), hydroxypropylmethylcellulose (K4M, K10), hydroxypropylcellulose (high viscosity grade-HF), polyethylene oxide and the like. A modified release material may conveniently be present in the range of 10-50% wt. of the tablet. An exemplary modified release tablet of Talabostat or a pharmaceutically acceptable salt thereof is given below in Table 3:

TABLE 3

| Formulation content | Amount (w/w %) |
|---|---|
| Talabostat as an API (available as | 0.01-2 |
| Talabostat mesylate) | |
| Polyvinylpyrrolidone (K90) or | 10-50 |
| hydroxypropylmethylcellulose (K4M, | |
| K10) or hydroxypropylcellulose (high | |
| viscosity grade-HF) or polyethylene oxide | |
| as a modified release material | |
| Sodium starch glycolate or crospovidone | 0-10 |
| as a disintegrant | |
| Magnesium stearate or stearic acid as a | 0.1-10 |
| lubricant | |
| Citric acid or phosphoric acid as a pH | 0-15 |
| modifying agent | |
| Lactose as a filler | 30-90 |

In some preferred embodiments, the amount of Talabostat in a unit dose is about 50 micrograms per tablet, about 100 micrograms per tablet, about 200 micrograms per tablet, about 300 micrograms per tablet, about 400 micrograms per tablet, about 500 micrograms per tablet, about 600 micrograms per tablet, about 700 micrograms per tablet, about 800 micrograms per tablet.

Various methods can be used for manufacturing tablets of Talabostat or a pharmaceutically acceptable salt thereof for use in the treatment regimen of this disclosure. One process includes dissolving Talabostat in a suitable solvent (with or without binder) and this solution is distributed uniformly over filler particles (which may contain other materials) to form agglomerated particles/granules. Wet granulation, coating or spraying processes can also be used. Granules may be appropriately sized or may be further processed by a dry granulation/slugging/roller compaction method followed by a milling step to achieve suitable granules of specific particle size distribution. The sized granules may be further blended with other components and/or and then lubricated in a suitable blender and compressed into tablets of specific dimensions using appropriate tooling. Coating of the tablets, where appropriate, may be performed using conventional methods and standard equipment.

### Kits

In some embodiments, the kit includes a formulation comprising Talabostat or pharmaceutically acceptable salt thereof and a formulation comprising Pembrolizumab with or without instructions for their use. The combined therapeutics can be manufactured and/or formulated by the same or different manufacturers. The combination therapeutics may thus be entirely separate pharmaceutical dosage forms or pharmaceutical compositions that are also sold independently of each other. In embodiments, instructions for their combined use are provided: (i) prior to release to physicians (e.g. in the case of a “kit of part” comprising a first therapeutic agent and the other therapeutic agent); (ii) by the physicians themselves (or under the guidance of a physician) shortly before administration; (iii) the patient themselves by a physician or medical staff.

In one example, a single bolus dose may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the disclosure are dictated by and directly dependent on the unique characteristics of the active compound and the particular therapeutic or prophylactic effect to be achieved.

In some embodiments, the kit comprises a package insert comprising instructions for using Talabostat or pharmaceutically acceptable salt thereof and Pembrolizumab to treat or delay progression of cancer in a subject or to enhance immune function of a subject having cancer. In some embodiments, the kit comprises Talabostat or pharmaceutically acceptable salt thereof and Pembrolizumab and package insert comprising instructions for using the same to treat or delay progression of cancer in a subject or to enhance immune function of a subject having cancer. In some embodiments, the kit comprises Talabostat or pharmaceutically acceptable salt thereof and Pembrolizumab, and a package insert comprising instructions for using the same to treat or delay progression of cancer in a subject or to enhance immune function of a subject having cancer.

In some embodiments, the kit comprises a container that includes, but is not limited to bottles, vials (e.g., dual chamber vials), syringes (such as single or dual chamber syringes) and test tubes. The container may be formed from a variety of materials such as glass or plastic. In some embodiments, the kit may comprise a label (e.g., on or associated with the container) or a package insert. The label or the package insert may indicate that the compound contained therein may be useful or intended for treating or delaying progression of cancer in a subject or for enhancing immune function of a subject having cancer. The kit may further comprise other materials desirable from a commercial and user standpoint, including other buffers, diluents, filters, needles, and syringes.

### Outcomes

Patients afflicted with prostate cancer (e.g. SCNC, NEPC, tNEPC, CrPC, mCrPC) administered a 0.3 mg twice daily dose of Talabostat or a pharmaceutically acceptable salt thereof and an effective amount of Pembrolizumab according to the treatment regimen disclosed herein preferably experience improvement in at least one sign of cancer. In some embodiments, improvement may be measured by a reduction in the quantity and/or size of measurable tumor lesions. In another embodiment, lesions can be measured using X-rays or CT or MRI scans. In another embodiment, cytology or histology can be used to evaluate responsiveness to the therapy. In another embodiment, extension of progression free survival and/or overall survival may be provided to a patient afflicted with prostate cancer (e.g. SCNC, NEPC, tNEPC, CrPC). In another embodiment, extension of progression free survival and/or overall survival may be provided to a patient afflicted with advanced solid cancer.

In some embodiments, the anti-tumor response is a tumor specific response, a clinical response, a decrease in tumor size/volume, a decrease in tumor specific biomarkers, increase in anti-tumor cytokines or a combination thereof.

In some embodiments, the clinical response is a decreased tumor growth and/or a decrease in tumor size. In some embodiments, the initiating, sustaining or enhancing an anti-tumor immune response is for the treatment of cancer.

In some embodiments, the anti-tumor response is inhibiting tumor growth, inducing tumor cell death, tumor regression, preventing or delaying tumor recurrence, tumor growth, tumor spread or tumor elimination.

In some embodiments, the anti-tumor response is reduction in metastasis, delay in metastasis or prevention of metastasis. In some embodiments, the anti-tumor response is prevention of metastasis.

In some embodiments, the tumor response is a decrease in the number of tumor cells. In some embodiments, the tumor response is a decreased rate in tumor growth. In some embodiments, the tumor response is a block in the dipeptidyl peptidase enzyme activity. In some embodiments, the tumor response is an induction of proinflammatory cytokine response and a cytotoxic T cell response.

The treatment regimen described herein may result in an inhibition of tumor size more than about 10%, more than about 20%, more than about 21%, more than about 22%, more than about 23%, more than about 24%, more than about 25%, more than about 26%, more than about 27%, more than about 28%, more than about 29%, more than about 30%, more than about 31%, more than about 32%, more than about 33%, more than about 34%, more than about 35%, more than about 36%, more than about 37%, more than about 38%, more than about 39%, more than about 40%, more than about 41%, more than about 42%, more than about 43%, more than about 44%, more than about 45%, more than about 46%, more than about 47%, more than about 48%, more than about 49%, more than about 50%, more than about 51%, more than about 52%, more than about 53%, more than about 54%, more than about 55%, more than about 56%, more than about 57%, more than about 58%, more than about 59%, more than about 60%, more than about 61%, more than about 62%, more than about 63%, more than about 64%, more than about 65%, more than 66%, more than 67%, more than 68%, more than 69%, more than about 70%, more than about 71%, more than about 72%, more than about 73%, more than about 74%, more than about 75%, more than about 76%, more than about 77%, more than about 78%, more than about 79%, more than about 80%, more than about 81%, more than about 82%, more than about 83%, more than about 84%, more than about 85%, more than about 86%, more than about 87%, more than about 88%, more than about 89%, more than about 90%, more than about 91%, more than about 92%, more than about 93%, more than about 94%, more than about 95%, more than about 96%, more than about 97%, more than about 98%, more than about 99% up to about 100%.

In some embodiments, the regimen and methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 95%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

In some embodiments, the regimen or methods provided herein can provide for 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 95%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the risk of developing a metastasis or the risk of developing an additional metastasis in a patient having prostate cancer (e.g. SCNC, NEPC, tNEPC, CrPC, mCrPC and adenocarcinoma).

In some embodiments, the treatment regimen or methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In some embodiments, patients afflicted with prostate cancer (e.g. SCNC, NEPC, tNEPC, CrPC, adenocarcinoma) administered a 0.3 mg twice daily dose of Talabostat or a pharmaceutically acceptable salt thereof on one or more days of a treatment cycle, and an effective amount of Pembrolizumab according to the treatment regimen disclosed herein may exhibit a complete response (CR), a partial response (PR), stable disease (SD), immune-related complete disease (irCR), immune-related partial response (irPR), or immune-related stable disease (irSD). In another embodiment, patients afflicted with advanced solid cancer administered Talabostat or a pharmaceutically acceptable salt thereof and Pembrolizumab according to the treatment regimen disclosed herein may exhibit a complete response (CR), a partial response (PR), stable disease (SD), immune-related complete disease (irCR), immune-related partial response (irPR), or immune-related stable disease (irSD).

In one embodiment, the patients afflicted with prostate cancer administered according to the treatment regimen achieves a stable disease response or better, as measured by RECIST 1.1.

In another embodiment, the patients afflicted with prostate cancer administered according to the treatment regimen achieves a partial disease response or better, as measured by RECIST 1.1.

In another embodiment the patients afflicted with prostate cancer administered according to the treatment regimen achieves a complete response or better, as measured by RECIST 1.1.

The term “complete response” as used herein refers to disappearance of all target lesions. Any pathological lymph nodes (whether target or nontarget) must have reduction in short axis to <10 mm.

The term “partial response” as used herein refers to at least a 30% decrease in the sum of diameters of target lesions, taking as reference the baseline sum diameters.

The term “stable disease” as used herein refers to neither sufficient shrinkage from the baseline study to qualify for PR nor sufficient increase to qualify for PD, taking as reference the smallest sum diameters while on study. The term “progressive disease” as used herein refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of 1 or more new lesions is also considered progression).

In another embodiment, the patients afflicted with an advanced solid tumor administered according to the treatment regimen achieves a complete or partial disease response or better, as measured by RECIST 1.1.

In another embodiment, the patients afflicted with advanced solid tumor administered according to the treatment regimen achieves a complete response or better, as measured by RECIST 1.1.

In another aspect, the subject experiences less treatment-related adverse events (TRAEs) relative to a subject with same cancer administered a single 0.6 mg daily dose of Talabostat. TRAEs contemplated within the scope of this invention are readily apparent to a person of ordinary skill in the art, and include, but are not limited to hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, rhabdomyolysis, myalgia, constipation, hypocalcemia, neutropenia, febrile neutropenia, anemia, leukopenia, pancytopenia, lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest. In some embodiments, the subject experiences no TRAEs.

In some embodiments, the subject achieves about a 50% or greater prostate-specific antigen (PSA) decline from baseline by about week 12 of treatment. For example, the subject achieves about a 50%, about a 51%, about a 52%, about a 53%, about a 54%, about a 55%, about a 56%, about a 57%, about a 58%, about a 59%, about a 60%, about a 61%, about a 62%, about a 63%, about a 64%, about a 65%, about a 66%, about a 67%, about a 68%, about a 69%, about a 70%, about a 71%, about a 72%, about a 73%, about a 74%, about a 75%, about a 76%, about a 77%, about a 78%, about a 79%, about a 80%, about a 81%, about a 82%, about a 83%, about a 84%, about a 85%, about a 86%, about a 87%, about a 88%, about a 89%, about a 90%, about a 591%, about a 92%, about a 93%, about a 94%, about a 95%, about a 96%, about a 97%, about a 98%, about a 99%, or about a 100% PSA decline from baseline by week 12 of treatment. The about 50% or greater PSA decline from baseline may occur within about 1 week, about 2 weeks, about 3 weeks, about 4 weeks, about 5 weeks, about 6 weeks, about 7 weeks, about 8 weeks, about 9 weeks, about 10 weeks, about 11 weeks, or within about 12 weeks.

In some embodiments, the subject experiences an increase in pro-inflammatory cytokines relative to a subject that is administered a single 0.6 mg daily dose of Talabostat. Pro-inflammatory cytokines contemplated within the scope of this invention are readily apparent to a person of ordinary skill in the art, and include, but are not limited to IL-6, IL-8, IL-18, IFN-γ, and IL-1β. In some embodiments, the pro-inflammatory cytokines are one or more of IL-18 and IFN-γ. In some embodiments, wherein the maximum increase in cytokines is observed at day 14 of continuous dosing.

In some embodiments, patients afflicted with prostate cancer (e.g. SCNC, NEPC, tNEPC, CrPC, adenocarcinoma) administered a 0.3 mg twice daily dose of Talabostat or a pharmaceutically acceptable salt thereof on one or more days of a treatment cycle and an effective amount of Pembrolizumab on day 1 of the treatment cycle, according to the treatment regimen disclosed herein may experience tumor shrinkage and/or decrease in growth rate, i.e., suppression of tumor growth. In another embodiment, unwanted cell proliferation may be reduced or inhibited.

In some embodiments, patients afflicted with advanced solid cancer administered a 0.3 mg twice daily dose of Talabostat or a pharmaceutically acceptable salt thereof on one or more days of a treatment cycle, and an effective amount of Pembrolizumab on day 1 of the treatment cycle according to the treatment regimen disclosed herein may experience tumor shrinkage and/or decrease in growth rate, i.e., suppression of tumor growth. In another embodiment, unwanted cell proliferation may be reduced or inhibited.

The term “advanced solid cancer” refers to a cancer that is not expected to be cured with available therapies. The objective for treatment in advanced cancers is to control the disease for as long as possible.

In yet another embodiment, one or more of the following may occur in patients afflicted with prostate cancer (e.g. SCNC, NEPC, tNEPC, CrPC, adenocarcinoma) administered 0.3 mg twice daily dose of Talabostat or a pharmaceutically acceptable salt thereof on one or more days of a treatment cycle and about 200 mg of Pembrolizumab on day 1 of the treatment cycle according to the treatment regimen disclosed herein: the number of cancer cells may be reduced; tumor size may be reduced; cancer cell infiltration into peripheral organs may be inhibited, retarded, slowed, or stopped; tumor metastasis may be slowed or inhibited; tumor growth may be inhibited; recurrence of tumor may be prevented or delayed; one or more of the symptoms associated with cancer may be alleviated.

In some embodiments, patients afflicted with advanced solid cancer may experience one or more of the following after administration of Talabostat and Pembrolizumab: the number of cancer cells may be reduced; tumor size may be reduced; cancer cell in filtration into peripheral organs may be inhibited, retarded, slowed, or stopped; tumor metastasis may be slowed or inhibited; tumor growth may be inhibited; recurrence of tumor may be prevented or delayed; one or more of the symptoms associated with cancer may be alleviated.

In some embodiments, patients afflicted with prostate cancer (e.g. SCNC, NEPC, tNEPC, CrPC, adenocarcinoma) administered a 0.3 mg twice daily dose of Talabostat or a pharmaceutically acceptable salt thereof on one or more days of a treatment cycle and about 200 mg of Pembrolizumab on day 1 of the treatment cycle according to the treatment regimen disclosed herein may exhibit at least one therapeutic effect selected from the group consisting of reduction in size of a tumor, reduction in number of metastatic lesions appearing over time, complete remission, partial remission, or stable disease.

In some embodiments, the patient afflicted with one or more solid tumors (e.g. an advanced solid tumor) administered a 0.3 mg twice daily dose of Talabostat or a pharmaceutically acceptable salt thereof on one or more days of a treatment cycle and about 200 mg of Pembrolizumab on day 1 of the treatment cycle according to the treatment regimens disclosed herein may exhibit at least one therapeutic effect selected from the group consisting of reduction in size of a tumor, reduction in number of metastatic lesions appearing over time, complete remission, partial remission, or stable disease.

In some embodiments, the treatment regimen herein may produce a comparable clinical benefit rate (CBR=CR+PR+SD≥6 months) better than that achieved by Talabostat or a pharmaceutically acceptable salt thereof, or Pembrolizumab alone.

In some embodiments, the improvement of clinical benefit rate achieved using the treatment regimen of the present disclosure may be about 20%, 30%, 40%, 50%, 60%, 70%, 80% or more compared to treatment using Talabostat or Pembrolizumab alone.

In some embodiments, the treatment regimen of the present disclosure may result in the CD8+ T cells in the subject having enhanced priming, activation, proliferation and/or cytolytic activity when compared to administration of Talabostat or a pharmaceutically acceptable salt thereof, or Pembrolizumab alone.

In some embodiments, the number of CD4+ and/or CD8+ T cells is elevated relative to prior to administration of the combination according to the treatment regimen described herein.

In some embodiments, the activated CD4+ and/or CD8+ T cells is characterized by γ-IFN+ producing CD4+ and/or CD8+ T cells and/or enhanced cytolytic activity relative to prior to the administration of the combination according to the treatment regimen described herein.

In some embodiments, the CD4+ and/or CD8+ T cells exhibit increased release of cytokines selected from the group consisting of G-CSF, MCP-1, Eotaxin, IFN-γ, KC, TNF-α and interleukins (IL-5, IL-6, IL-113, IL-12p70, IL 18).

In some embodiments, the CD4+ and/or CD8+ T cell is an effector memory T cell. In some embodiments, the CD4+ and/or CD8+ effector memory T cell is characterized by γ-IFN+ producing CD4+ and/or CD8+ T cells and/or enhanced cytolytic activity.

In some embodiments, the serum levels of cytokine IL-18 and/or chemokine GM-CSF, G-CSF in the subject are increased. in the presence of combination of Talabostat or a pharmaceutically acceptable salt thereof and Pembrolizumab when used according to the treatment regimen described herein as compared to single agent administration.

In some embodiments, the cancer has elevated levels of T-cell infiltration when a combination of Talabostat or a pharmaceutically acceptable salt thereof, and Pembrolizumab is used according to the treatment regimen described herein, when compared to administration of Talabostat or Pembrolizumab alone.

In some embodiments, the cancer has suppressed/decreased levels of T-regulatory cells in the presence of a combination of Talabostat or a pharmaceutically acceptable salt thereof and Pembrolizumab when used according to the treatment regimen described herein, when compared to administration of Talabostat or a pharmaceutically acceptable salt thereof, or Pembrolizumab alone. In some embodiments, the cancer has increased levels of NK cells and macrophages in the presence of combination of Talabostat or a pharmaceutically acceptable salt thereof and Pembrolizumab when used according to the treatment regimen described herein, when compared to administration of Talabostat or a pharmaceutically acceptable salt thereof, or Pembrolizumab alone.

With respect to target lesions, responses to the treatment regimen described herein may include: Complete response (CR), Partial Response (PR), Progressive Disease (PD), Stable Disease (SD), Immune-related Complete Response (irCR), Immune-related Partial Response (irPR), Immune-related Progressive Disease (irPD) and Immune-related Stable Disease (irSD).

With respect to non-target lesions, responses to the treatment regimen described herein may include: Complete Response (CR), Progressive Disease (PD), Immune-related Complete Response (irCR) and Immune-related Progressive Disease (irPD).

In one embodiment, the patient treated exhibits a complete response (CR), a partial response (PR), stable disease (SD), immune-related complete disease (irCR), immune-related partial response (irPR), or immune-related stable disease (irSD). In another embodiment, the patient treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

Tumor response evaluation is performed using the following RECIST definitions.

The term “measurable disease” as used herein refers to presence of at least one measurable lesion. Measurable lesions must be accurately measured in at least 1 dimension (longest diameter in the plane of the measurement to be recorded) with a minimum size of:
10 mm by CT scan (CT scan slice thickness no greater than 5 mm).
10 mm caliper measurement by clinical exam (lesions which cannot be accurately measured with calipers should be recorded as non-measurable).
20 mm by chest X-ray.

The term “malignant lymph nodes” is defined to be pathologically enlarged and measurable, if a lymph node was ≥15 mm in short axis when assessed by CT scan (CT scan slice thickness recommended to be no greater than 5 mm). At baseline and in follow-up, only the short axis was measured and followed.

Non-measurable lesions were all other lesions, including small lesions (longest diameter <10 mm or pathological lymph nodes with ≥10 to <15 mm short axis), as well as non-measurable lesions.

The term “target lesions” refers to when more than one lesion is present at baseline all lesions up to a maximum of five lesions total representative of all involved organs are identified as target lesions. All other lesions including pathological lymph nodes are identified as “non-target lesions” and are also recorded at baseline.

The duration of overall response was measured from the time measurement criteria was first met for CR/PR (whichever was first recorded) until the first date that recurrent or PD was objectively documented (taking as reference for PD the smallest measurements recorded on study).

The duration of overall CR is measured from the time measurement criteria were first met for CR until the first date that recurrent disease was objectively documented.

Stable disease was measured from the start of the treatment until the criteria for progression were met, taking as reference the smallest sum on study (if the baseline sum was the smallest, this was the reference for calculation of PD).

For the evaluation of non-target lesions, the following definitions of the criteria were used to determine tumor response:

The term “complete response” or (CR) as used herein refers to disappearance of all non-target lesions and normalization of tumor marker level. All lymph nodes must be non-pathological in size (<10 mm short axis).

The term “non-CR/non-PD” as used herein refers to persistence of one or more non-target lesion(s) and/or maintenance of tumor marker level above the normal limits. The term “progressive disease” as used herein refers to unequivocal progression of existing non-target lesions.

The term “unequivocal progression” as used herein refers to an overall level of substantial worsening in non-target disease such that even in presence of SD or PR in target disease, the overall burden had increased sufficiently to merit discontinuation of therapy.

The term “not evaluable or NE” or “inevaluable” refers to when no imaging/measurement is done at all at a particular time point.

Radiographic PFS is defined as the time from the date of initiation of protocol therapy to date measurement criteria were first met for PD by RECIST 1.1/PCWG3 criteria or death from any cause, whichever occurred first.

Progression Free Survival (PFS) is defined as the time from the date of initiation of protocol therapy to date measurement criteria were first met for PSA progression by PCWG3 criteria. Overall Survival: Survival time was the difference in days between the date of death and the first date of study treatment (+1 day).

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

At various places in the present specification, substituents of compounds of the disclosure are disclosed in groups or in ranges. It is specifically intended that the disclosure include each and every individual subcombination of the members of such groups and ranges.

### Specific Embodiments of the Present Disclosure

Embodiment 1: A treatment regimen for treating prostate cancer in a subject in need thereof, comprising administering to said subject, about 0.3 mg twice daily dose of Talabostat or a pharmaceutically acceptable salt thereof on one or more days of a treatment cycle and an effective amount of Pembrolizumab on day 1 of the treatment cycle.

Embodiment 2: A method of treating prostate cancer in a subject, comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle, and wherein the prostate cancer is selected from the group consisting of small cell neuroendocrine prostate cancer (SCNC), neuroendocrine prostate cancer (NEPC), treatment emergent neuroendocrine prostate cancer (tNEPC), castration resistant prostate cancer (CrPC), metastatic castration resistant prostate cancer (mCrPC), and adenocarcinoma type prostate cancer.

Embodiment 3: A method of enhancing an immune response in a subject suffering from prostate cancer (e.g. SCNC, NEPC, tNEPC, CrPC, mCrPC, adenocarcinoma), the method comprising administering to said subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 4: A method of enhancing an innate immune response in a subject afflicted with prostate cancer (e.g. SCNC, NEPC, tNEPC, CrPC, mCrPC, adenocarcinoma), the method comprising administering to said subject, Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and, wherein the enhanced innate immune response is associated with increased tumoricidal natural killer cells and macrophages, as well as the activity of NK cells and CD8+ T cells.

Embodiment 5: A method of enhancing an innate immune response in a subject with prostate cancer (e.g. SCNC, NEPC, tNEPC, CrPC, mCrPC, adenocarcinoma), the method comprising administering to said subject, Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and, wherein the enhanced innate immune response is associated with suppression of T-regulatory cells

Embodiment 6: A method for treating, delaying progression or preventing or delaying tumor recurrence, tumor growth or spread of tumor in a subject afflicted with prostate cancer (e.g. SCNC, neuroendocrine prostate cancer (NEPC), treatment emergent neuroendocrine prostate cancer (tNEPC), castration resistant prostate cancer (CrPC), metastatic castration resistant prostate cancer (mCrPC), adenocarcinoma type prostate cancer), the method comprising administering to the subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 7: A method of enhancing immune function in a subject afflicted with prostate cancer (e.g. SCNC, neuroendocrine prostate cancer (NEPC), treatment emergent neuroendocrine prostate cancer (tNEPC), castration resistant prostate cancer (CrPC), metastatic castration resistant prostate cancer (mCrPC), adenocarcinoma type prostate cancer), the method comprising administering to the subject, Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 8: A method for initiating, sustaining or enhancing an anti-tumor immune response in a subject afflicted with prostate cancer (e.g. SCNC, neuroendocrine prostate cancer (NEPC), treatment emergent neuroendocrine prostate cancer (tNEPC), castration resistant prostate cancer (CrPC), metastatic castration resistant prostate cancer (mCrPC), adenocarcinoma type prostate cancer), the method comprising administering to the subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 9: A method for reducing the treatment related adverse effects (TRAEs) in a subject afflicted with prostate cancer (e.g. SCNC, neuroendocrine prostate cancer (NEPC), treatment emergent neuroendocrine prostate cancer (tNEPC), castration resistant prostate cancer (CrPC), metastatic castration resistant prostate cancer (mCrPC), adenocarcinoma type prostate cancer), comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 10. A method of treating an advanced solid tumor (e.g. castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma), in a subject in need thereof, the method comprising administering to the subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 11: A method of enhancing an immune response in a subject suffering from an advanced solid tumor (e.g. castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma), the method comprising administering to said subject a regimen comprising Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 12: A method of enhancing an innate immune response in a subject afflicted with advanced solid tumor (e.g. castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma), the method comprising administering to said subject, Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and wherein the enhanced innate immune response is associated with increased tumoricidal natural killer cells and macrophages, as well as the activity of NK cells and CD8+ T cells.

Embodiment 13: A method of enhancing an innate immune response in a subject with an advanced solid tumor (e.g. castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma), the method comprising administering to said subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and wherein the enhanced innate immune response is associated with suppression of T-regulatory cells.

Embodiment 14: A method for treating, delaying progression or preventing or delaying tumor recurrence, tumor growth or spread of tumor in a subject afflicted with an advanced solid tumor (e.g. castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma), the method comprising administering to the subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 15: A method of enhancing immune function in a subject afflicted with an advanced solid tumor (e.g. castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma), the method comprising administering to the subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 16: A method for initiating, sustaining or enhancing an anti-tumor immune response in a subject afflicted with an advanced solid tumor (e.g. castrate resistant prostate cancer, endometrial cancer, dedifferentiated liposarcoma, basal cell carcinoma, leiomyosarcoma, squamous cell carcinoma of unknown primary, mucosal melanoma, skin melanoma, pleomorphic sarcoma, colorectal/colon cancer, astrocytoma, triple negative breast cancer, uterine sarcoma, uveal melanoma), the method comprising administering to the subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 17: A method for reducing the treatment related adverse effects (TRAEs) in a subject afflicted with an advanced solid tumor (e.g. castrate resistant prostate cancer, endometrial cancer, dedifferentiated liposarcoma, basal cell carcinoma, leiomyosarcoma, squamous cell carcinoma of unknown primary, skin melanoma, mucosal melanoma, pleomorphic sarcoma, colorectal/colon cancer, astrocytoma, triple negative breast cancer, uterine sarcoma, uveal melanoma), comprising administering to a subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 18: The treatment regimen or method of treatment according to any of Embodiments 1-17, wherein Talabostat or a pharmaceutically acceptable salt thereof and Pembrolizumab are administered to the subject in one or more (e.g. 1, 2, 3, 4, 5, 6 or more) treatment cycles, where each treatment cycle is of about 21 days duration.

Embodiment 19: The treatment regimen or method of treatment according to any of Embodiments 1-18, wherein after cessation of treatment the subject maintains a sustained response to progression of prostate cancer or an advanced solid tumor.

Embodiment 20: The treatment regimen or method of treatment according to Embodiment 18 or 19, wherein for each treatment cycle Talabostat or a pharmaceutically acceptable salt thereof is administered on each of days 1 to 14 and Pembrolizumab is administered on day 1.

Embodiment 21: The treatment regimen or method of treatment according to any of Embodiments 1-20, wherein Talabostat or a pharmaceutically acceptable salt thereof is administered orally (e.g. as a tablet formulation).

Embodiment 22: The treatment regimen or method of treatment according to any of Embodiments 1-20, wherein Pembrolizumab is administered by injection (e.g. intravenously).

Embodiment 23: The treatment regimen or method of treatment according to any of Embodiments 1-22, wherein Pembrolizumab is administered at a dose of from about 1 mg/kg to about 10 mg/kg per day.

Embodiment 24: The treatment regimen or method of treatment according to any of Embodiments 1-23, wherein Talabostat or a pharmaceutically acceptable salt thereof is administered at a dose of about 0.3 mg in the morning and about 0.3 mg in the evening.

Embodiment 25: The treatment regimen or method of treatment according to any of Embodiments 1-24, wherein Pembrolizumab is administered at a total dose of from about 100 mg to about 500 mg per day (e.g. about 200 mg per day).

Embodiment 26: The treatment regimen or method of treatment according to any of Embodiments 1-24, wherein the total daily dose of Talabostat or a pharmaceutically acceptable salt thereof in cycle 1 is lower than the total daily dose of Talabostat or a pharmaceutically acceptable salt thereof in one or more subsequent cycles.

Embodiment 27: The treatment regimen or method according to any of Embodiments 1 to 17, wherein the subject is administered Talabostat or a pharmaceutically acceptable salt thereof at a dose of about 0.2 mg twice daily for one or more consecutive days beginning on day 1 of the first treatment cycle.

Embodiment 28: The treatment regimen or method according to any of Embodiments 1 to 17, wherein the subject is administered Talabostat or a pharmaceutically acceptable salt thereof at a dose of about 0.2 mg twice daily on days 1-7 of the first treatment cycle followed by about 0.3 mg twice daily on days 8-14 of the first treatment cycle.

Embodiment 29: The treatment regimen or method according to any of Embodiments 1 to 17, wherein the subject is administered Talabostat or a pharmaceutically acceptable salt thereof at a dose of about 0.3 mg twice daily on days 1-3 of the first treatment cycle followed by rest period (day 4 to 7) and then about 0.3 mg twice daily (on days 8-11) of the first treatment cycle.

Embodiment 30: The treatment regimen or method according to Embodiments 1 to 9, wherein the prostate cancer is adenocarcinoma type prostate cancer.

Embodiment 31: The treatment regimen or method of treatment according to Embodiment 30, wherein the subject with adenocarcinoma prostate cancer was treated with at least 1 but no more than 2 second generation AR pathway target inhibitors.

Embodiment 32: The treatment regimen or method of treatment according to any of Embodiments 1 to 9, wherein the subject has an Eastern Cooperative Oncology Group (ECOG) performance status of 0-2.

Embodiment 33: The treatment regimen or method of treatment according to any of Embodiments 1 to 9, wherein the subject with adenocarcinoma prostate cancer has a serum testosterone <50 ng/dL during screening.

Embodiment 34: The treatment regimen or method according to any of Embodiments 1 to 17, wherein the subject experiences less treatment-related adverse events (TRAEs) relative to a subject with same caner and administered a single 0.6 mg once daily dose of Talabostat

Embodiment 35: The treatment regimen or method according to Embodiment 34, wherein the subject experiences no TRAEs.

Embodiment 36: The treatment regimen or method according to Embodiment 34 or 35, wherein the TRAE are one or more of hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, rhabdomyolysis, myalgia, constipation, hypocalcemia, neutropenia, febrile neutropenia, anemia, leukopenia, pancytopenia, and lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest.

Embodiment 37: The treatment regimen or method according to any of preceding Embodiments, wherein the subject achieves a stable disease response or better, as measured by RECIST 1.1.

Embodiment 38: The treatment regimen or method according to any of preceding Embodiments, wherein the subject achieves a partial response or better, as measured by RECIST 1.1.

Embodiments 39: The treatment regimen or method of any of the preceding embodiments, wherein the subject achieves a complete response, as measured by RECIST 1.1.

Embodiment 40: The treatment regimen or method according to any of preceding embodiments, wherein the subject achieves a 50% or greater prostate-specific antigen (PSA) decline from baseline by week 12 of treatment.

Embodiment 41: The treatment regimen or method according to any of preceding embodiments, wherein the subject experiences an increase in pro-inflammatory cytokines relative to a subject that is administered a single 0.6 mg once daily dose of Talabostat.

Embodiment 42: The method according to Embodiment 41, wherein the pro-inflammatory cytokines are one or more of IL-18 and IFN-γ.

Embodiment 43: The treatment regimen or method according to Embodiment 41, wherein the maximum increase in cytokines is observed at day 14 of continuous dosing.

Embodiment 44: The treatment regimen or method according to any of Embodiments 10 to 17, wherein the subject was not previously treated with PD-1/PD-L1 or CTLA-4 antibodies.

Embodiment 45: The treatment regimen or method according to any of Embodiments 10 to 16, wherein the subject has relapsed or progressed with PD-1/PD-L1 or CTLA-4 antibodies.

Embodiment 46: The treatment regimen or method of treatment according to any of Embodiments 1-27, comprising administering Talabostat mesylate.

Embodiment 47: A treatment regimen for treating prostate cancer ((e.g. SCNC, NEPC, tNEPC, CrPC, mCrPC, adenocarcinoma), in a subject in need thereof, the regimen comprising administering to the subject Talabostat mesylate and Pembrolizumab in one or more treatment cycles, where each treatment cycle is of about 21 days duration, and for each treatment cycle Talabostat is administered on each of days 1 to 14 and Pembrolizumab is administered on day 1, wherein Talabostat mesylate is administered orally as one or more tablets to provide a total daily dose of Talabostat of about 0.6 mg in divided doses and Pembrolizumab is administered as a single intravenous injection to provide a dose of from about 100 mg to about 500 mg per day.

Embodiment 48. The treatment regimen according to Embodiment 47, wherein Talabostat or a pharmaceutically acceptable salt thereof is administered orally in the morning and evening.

Embodiment 49. The treatment regimen according to Embodiment 47, wherein the subject experiences less treatment-related adverse events (TRAEs) relative to a subject that is administered a single 0.6 mg once daily dose of Talabostat.

Embodiment 50. The treatment regimen according to Embodiment 47, wherein the subject experiences no TRAEs.

Embodiment 51. The treatment regimen according to Embodiment 47, wherein the subject has a small cell neuroendocrine prostate cancer and has received at least one prior cytotoxic chemotherapy.

Embodiment 52. The treatment regimen according to Embodiment 47, wherein the subject has a measurable disease as per RECIST 1.1 or iRECIST.

Embodiment 53. The treatment regimen according to Embodiment 47, wherein the subject has a detectable bone metastases by whole body bone scintigraphy.

Embodiment 54. The treatment regimen according to Embodiment 47, wherein the subject after the administration meets the CTC response as per Veridex assay.

Embodiment 55. The treatment regimen according to Embodiment 47, wherein the subject achieves a stable disease response or better as per RECIST 1.1.

Embodiment 56. The treatment regimen according to Embodiment 47, wherein the subject achieves a complete or a partial response or better as per RECIST 1.1

### EXAMPLES

Example 1: A Phase 1b/2 Study of Talabostat mesylate, a Small Molecule Inhibitor of Dipeptidyl Peptidases (DPP), Administered in Combination with the Anti-Programmed Cell Death 1 (PD-1) Monoclonal Antibody Pembrolizumab (PEMBRO; Keytruda®) in Patients with Small Cell Neuroendocrine Prostate Cancer (SCNC; NEPC) or Adenocarcinoma Phenotype.

Study Objectives: The purpose of the study was to assess the safety and efficacy of talabostat mesylate in combination with pembrolizumab in patients with metastatic castrate-resistant prostate cancer (mCRPC). In the Phase 1b portion, increasing dose levels of talabostat mesylate were explored sequentially in patients with mCRPC regardless of histology. In the efficacy portion of the study, patient assignment to 1 of 2 cohorts was based on histopathology.

Cohort A: Patients with any small cell/neuroendocrine (SCNC) features, either de novo or treatment-emergent including mixed SCNC.

Cohort B: Patients with adenocarcinoma and no evidence of small cell or neuroendocrine features on recent histopathology.

The following objectives applied to both cohorts, however, due to more aggressive nature of the disease for patients in Cohort A, the primary and secondary efficacy related objectives were assessed separately for each Cohort.

The primary objectives of the study were:
To estimate the composite response rate of the combination of Talabostat mesylate+Pembrolizumab in Cohort A and B respectively. Composite response rate is defined as achieving 1 or more of the following:
Objective response by Response Evaluation Criteria in Solid Tumors (RECIST) 1.1 criteria
Circulating tumor cell (CTC) conversion from >5/7.5 mL to <5/7.5 mL per Veridex assay by completion of Week 12 of protocol therapy
Greater than 50% prostate-specific antigen (PSA) decline from baseline by completion of Week 12 of protocol therapy.

The secondary objectives of the study were:
To estimate the median radiographic progression-free survival (rPFS) of the combination of Talabostat mesylate and Pembrolizumab in Cohort A and B.
To estimate the median PSA progression-free survival (PSA PFS) of the combination of Talabostat mesylate and Pembrolizumab in Cohort A and B.
To estimate the median overall survival (OS) of the combination of Talabostat mesylate and Pembrolizumab in Cohort A and B.
To estimate the median duration of response (DOR) of the combination of Talabostat mesylate and Pembrolizumab in Cohort A and B.
To continue to characterize the safety profile of Talabostat mesylate in combination with Pembrolizumab.
To assess population pharmacokinetics of Talabostat mesylate using sparse pharmacokinetic sampling.
To assess the pharmacodynamic profile of the combination of Talabostat mesylate and Pembrolizumab by measuring relevant effects on those cytokines previously shown to be modulated by Talabostat mesylate in humans.

Exploratory Objectives:
To determine the response rate by Immune Response Evaluation Criteria in Solid Tumors (iRECIST) criteria with Talabostat mesylate in combination with Pembrolizumab.
To evaluate the quantitative and qualitative effects of Talabostat mesylate in combination with Pembrolizumab on relevant immune effector cytokines and various immunological effector cells in blood and, whenever feasible, in tumor tissues.
To explore the predictive value of baseline programmed death-ligand 1 (PD-L1) tumor expression in metastatic tumor tissue and CTCs with subsequent clinical outcomes.
To explore the relationship between baseline tumor messenger ribonucleic acid (mRNA) immune profiling panel and clinical outcomes.

Study Design: This was an open-label, multi-centre, Phase 1b/2 study determined the composite response rate of talabostat mesylate administered orally and daily, combined with Pembrolizumab, in patients with mCRPC enrolled in stage 2 with either SCNC (Cohort A) or adenocarcinoma phenotype (Cohort B). The study assessed other efficacy parameters such as rPFS, PSA PFS, OS and DOR, as well as safety of the combined treatment.

The study consisted of the following two stages:

Lead-in Stage: in which the safety and tolerability of the combination of Talabostat mesylate administered on Days 1 to 14 of a 21-day cycle plus Pembrolizumab 200 mg administered intravenously (IV) on Day 1 every 21 days was assessed and confirmed in patients with metastatic castration-resistant prostate cancer (mCRPC). In Cohort 1, the initial dose level of Talabostat mesylate was 0.4 mg; if there were no safety concerns, this was escalated to a total daily dose of 0.6 mg. There was a 7-day rest period following the last dose of Talabostat mesylate and Day 1 of the subsequent cycle.

Efficacy stage (Simon 2-stage): in which patients were treated with talabostat mesylate combined with Pembrolizumab. Patients were assigned to 1 of 2 cohorts based on phenotype.
Cohort A: Patients with any small cell/neuroendocrine (SCNC) features, either de novo or treatment-emergent including mixed SCNC.
Cohort B: Patients with adenocarcinoma and no evidence of small cell or neuroendocrine features

During the Lead-in Stage, patients were observed for dose-limiting toxicity (DLT) during Cycle 1. Three patients were treated initially with 0.4 mg Talabostat mesylate plus Pembrolizumab:
If there were no DLTs in Cycle 1, the dose of Talabostat mesylate was escalated to total daily dose of 0.6 mg in the next cohort of 3 patients.
If ≥1 of the 3 original patients has a DLT in Cycle 1, after a discussion between the sponsor and the investigator, either 3 patients (if 1 patient experienced a DLT) or 6 to 9 patients (if 2 or 3 patients experienced a DLT) were added at the 0.4 mg Talabostat mesylate dose level
For this expanded 0.4 mg cohort:
If less than one-third of the patients experienced a DLT, consideration was given to dose escalation to 0.6 mg Talabostat mesylate plus Pembrolizumab;
If one-third of the patients experienced a DLT, the Efficacy Stage could commence;
If more than one-third of the patients experience a DLT, a discussion was held between the investigators and sponsors as to how to proceed.

Following dose escalation to 0.6 mg Talabostat mesylate plus Pembrolizumab in 3 patients:
If there are no DLTs at this dose level, the Efficacy Stage could commence.
If ≥1/3 patients had a DLT in Cycle 1, after a discussion between the sponsor and the investigator, 6 to 9 patients were added at the 0.6 mg Talabostat mesylate dose level; and consideration of an alternate dosing (e.g., split dose) schedule may be given.
For this additional cohort of 6 to 9 patients\
   If less than or equal to one-third of the patients experienced a DLT, the Efficacy Stage could commence.
   If more than one-third of the patients experienced a DLT, consideration was given to the use of 0.4 mg Talabostat mesylate total daily dose, or an intermediate dose, plus Pembrolizumab in the Efficacy Stage.

Patients in the Lead-In Stage who did not met the criteria for discontinuation were allowed to continue treatment. Once the recommended dosing regimen for Talabostat mesylate for the efficacy stage was determined, any patients still tolerating treatment in a lower Talabostat mesylate dose cohort had their Talabostat mesylate doses escalated to the recommended dosing regimen with the start of the next cycle at the investigator's discretion.

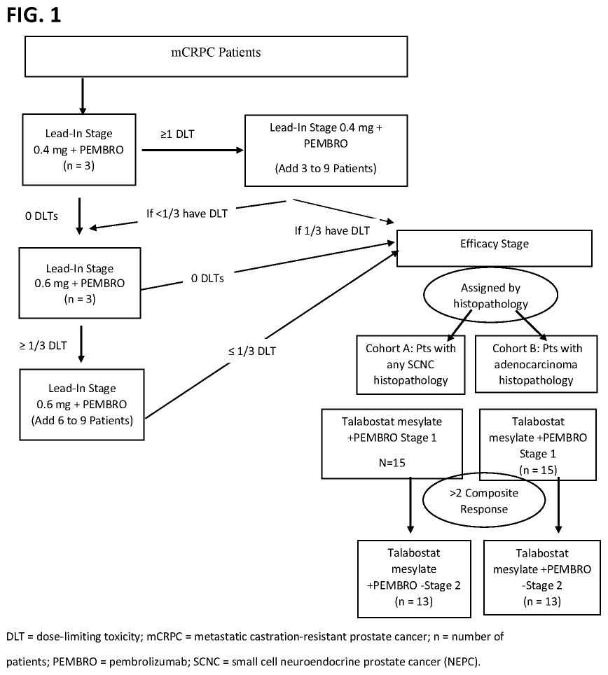

The study scheme is presented in FIG. 1.

A Safety Review Committee, which comprised of both investigators and sponsor representatives, reviewed the safety during the Lead-In Stage and during the trial before the Efficacy Stage began. If an intermediate dose was selected for the Efficacy Stage, the committee monitored the first 6 patients enrolled in the efficacy stage through Cycle 1.

All safety data from all patients enrolled in each cohort was reviewed to confirm any DLTs that were experienced and to determine enrolling the next cohort, as well as the Talabostat mesylate dosing regimen to be used in the Efficacy Stage. Unless doses were held because of adverse events (AEs), a patient must had received >70% of his Talabostat mesylate doses in Cycle 1 (e.g., ≥10 of 14 planned doses) with Pembrolizumab dosed on Day 1 of Cycle 1 to be eligible for DLT assessment.

Toxicities were assessed by the investigator using the National Cancer Institute Common Terminology Criteria for Adverse Events (NCI CTCAE), version 5. The relationship of an AE to combination therapy (i.e., attribution to Talabostat mesylate and/or Pembrolizumab) was to be assessed by the investigator using the criteria in the protocol.

A DLT was defined as any of the following AEs occurring during Cycle 1, regardless of investigator attribution to study treatment, unless the AE could be clearly and incontrovertibly attributed to an extraneous cause (e.g., PD) by the Principal Investigator PI:
Any Grade 4 laboratory abnormality, regardless of duration
Any Grade 3 non-hematologic AE, with the exceptions of Grade 3 nausea, vomiting, diarrhea, constipation, fever, fatigue, skin rash, or non-clinically significant laboratory abnormality that resolved to Grade <2 within 72 hours with optimal medical management.
Grade 3 thrombocytopenia with Grade >1 bleeding or requirement for platelet transfusion.
Grade 3 febrile neutropenia.
Grade 3 fever.
Grade 3 skin rash.
Laboratory abnormalities meeting Hy's law criteria (aspartate aminotransferase [AST] or alanine aminotransferase [ALT]>3×upper limit of normal [ULN] with concomitant total bilirubin>2×ULN).
Grade 3 transaminase (AST/ALT) elevation.
Any toxicity resulting in ≥30% held/skipped doses of Talabostat mesylate during Cycle 1.
Delay of Cycle 2 by ≥14 days due to toxicity.
Any other significant toxicity considered by the investigator and sponsor's medical representatives to be dose-limiting.

Efficacy Stage: After assessment of the safety and confirmation of the Talabostat mesylate/Pembrolizumab dose regimen used in the subsequent stage, the Efficacy Stage began. Eligible SCNC patients received Talabostat mesylate on Days 1 to 14 of a 21-day cycle plus Pembrolizumab 200 mg administered IV on Day 1 every 21 days.

Study Design Features (Both Stages): In both the Lead-In and Efficacy Stages, patients were screened for study eligibility within 28 days before the first study drug dose after provision of written informed consent. Patients who were determined to be eligible, based on screening assessments were enrolled in the study on Cycle (C)1, Day (D) 1 (Baseline, before the first dose of Talabostat mesylate).

During treatment, patients attended study center visits and had study evaluations performed as detailed in the Schedule of Assessments (Table 15). All study visits were conducted on outpatient basis but might be conducted on inpatient basis per the investigator's judgement.

All patients had pre-treatment (prior to study treatment dosing) imaging (computed tomography [CT] scan of chest/abdomen/pelvis or magnetic resonance imaging [MRI] for baseline tumor measurements, as well as bone scintigraphy [BS]). Patients with skin, subcutaneous or lymph node metastases may have also had tumor evaluations (including measurements, with a ruler) by means of physical examination. Patients with a history of central nervous system (CNS) malignant involvement or CNS symptoms had either CT or MRI imaging of the brain performed to assess active CNS malignancy.

Tumor measurements and disease response assessments (CT or MRI; BS) were also performed at the end of Cycle 3 (approximately 9 weeks after the first study treatment dose), and then approximately every 9 weeks thereafter until development of progressive disease (PD). For patients with evidence of disease control (stable disease or better) at Week 27, tumor measurements and disease response assessments might be performed less frequently (approximately every 12 weeks) thereafter. Tumor measurements and disease response assessments also were performed at the End of Treatment (EOT) visit.

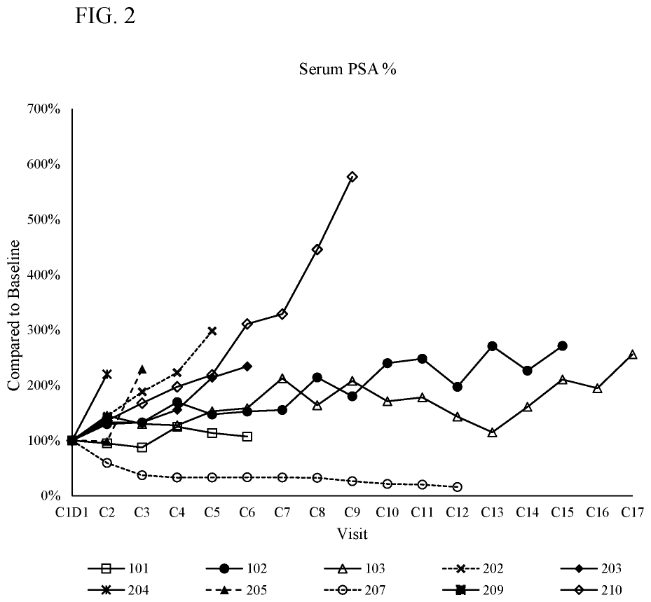

Additionally, measurement of serum PSA was performed on Day 1 of every treatment cycle. See FIG. 2.

Enumeration of CTCs by Veridex assay was performed at pre-dose (−2 hours) on C1D1, C2D1, C4D1, and every 3 cycles thereafter until the EOT visit.

Population pharmacokinetics of Talabostat mesylate was assessed using sparse pharmacokinetic sampling.

Patients continued to receive treatment until the development of radiographic progression by RECIST 1.1/Prostate Cancer Working Group 3 (PCWG3) criteria, unequivocal clinical progression, unacceptable toxicity, another discontinuation criterion was met, or closure of the study; no maximum duration of therapy had been set. Patients with PSA progression in the absence of radiographic or clinical progression should continue to receive protocol therapy.

Treatment might continue beyond the first radiographic progression if there was clinical benefit.

All remaining patient samples at the end of the study were de-identified with respect to the patient and moved into long-term storage for up to 5 years for future analysis, after which time the samples were destroyed.

Study Population

Approximately 13 patients with mCRPC were enrolled in the Lead-in Stage of the study. A total of between 30 and 56 patients were enrolled across 2 separate Cohorts in the Efficacy stage of the study with approximately 15 to 28 patients with SCNC and approximately 15 to 28 patients with adenocarcinoma phenotype.

Eligibility Criteria: All patients must satisfy the following inclusion and exclusion criteria to be eligible for entry into the trial.

Inclusion Criteria
1. Patient had an evidence of progressive, mCRPC, as defined by PCWG3 criteria.
   a. Patients with de novo small cell prostate cancer were not required to have received androgen deprivation therapy (ADT).
2. Progression during or following completion of at least 1 prior line of systemic therapy for locally advanced or metastatic prostate cancer.
3. Efficacy Stage only:

### For Cohort A (SCNC):

a. Patient had a histologic evidence of SCNC either with archival tissue or a fresh tumor biopsy obtained during Screening. Archival or fresh tumor biopsy tissue must be submitted for a central laboratory pathology review; however, enrollment could proceed if SCNC was determined by a local pathology review. (Central pathology review was optional for patients enrolled in the lead-in.)
b. Must be willing to undergo metastatic tumor biopsy during Screening. Requirement might be waived in patients without safely accessible lesion or for patients with evaluable archival metastatic tumor tissue
c. Patient had previously received at least 1 prior line of cytotoxic chemotherapy. Patients who either had refused chemotherapy or considered unsuitable for chemotherapy might be eligible following discussion with the sponsor.
d. Had measurable disease per RECIST 1.1 criteria.

### For Cohort B (Adenocarcinoma):

a. Patient had histologically or cytologically confirmed adenocarcinoma of the prostate without small cell neuroendocrine features.
b. Patients with soft tissue disease must provide a fresh core or excisional biopsy or archival tissue obtained ≤3 mo prior to study start from a site not previously irradiated for central pathology review; however enrollment might proceed if predominant adenocarcinoma without small cell neuroendocrine features was determined by local pathology review.
c. Had been treated with at least 1 but no more than 2 second generation AR pathway target agents (e.g., abiraterone acetate and/or enzalutamide or other next generation agent) and at least 1 regimen/line of taxane containing chemotherapy in the mCSPC or mCRPC setting. Patients with known actionable mutations should have progressed on applicable standard care targeted therapy or had documented intolerance to or be unsuitable for such therapy.
d. RECIST 1.1 measurable disease or detectable bone metastases by whole body bone scintigraphy.
4. Patient had serum testosterone <50 ng/dL during Screening except for those with de novo small cell prostate cancer.
a. Patients with treatment-emergent SCNC without a history of bilateral orchiectomy were required to remain on luteinizing hormone-releasing hormone (LHRH) analog during the course of protocol therapy except for patients with de novo small cell prostate cancer.
5. Patient had Eastern Cooperative Oncology Group (ECOG) performance status of 0-2.
6. Patient was aged ≥18 years.
7. Patient's acute toxic effects of previous anticancer therapy had resolved to ≤Grade 1 except for Grade 2-3 peripheral neuropathy or any grade of alopecia.
8. Patient had adequate baseline organ function, as demonstrated by the following:
a. Serum creatinine ≤1.5 times institutional upper limit of normal ULN or calculated creatinine clearance >50 mL/min;
b. Serum albumin ≥2.5 g/dL;
c. Total bilirubin ≤1.5×ULN;
d. Aspartate aminotransferase and ALT≤2.5×institutional ULN (patients with hepatic metastases must had AST/ALT≤5×ULN);
9. Patient had adequate baseline hematologic function, as demonstrated by the following:
a. Absolute neutrophil count (ANC)≥1.5×109/L.
b. Hemoglobin ≥8 g/dL and no red blood cell transfusions during the prior 14 days.
c. Platelet count ≥100×109/L and no platelet transfusions during the prior 14 days.
10. Male patients and their female partners of childbearing potential must agree and commit to use a barrier contraception (e.g., condom with spermicidal foam/gel/film/cream/suppository) throughout the duration of the study until at least 6 months following the last dose of study drug, in addition to their female partners using either an intrauterine device or hormonal contraception and continuing until at least 6 months following the last dose of study drug. This criterion might be waived for male patients who had a vasectomy >6 months before signing the informed consent form. In the United States, female partners of study participants were not required to use contraception as a condition of their partner's eligibility, but female partners with child bearing potential should consider use of effective methods of contraception for the duration of their male partner's study participation and for at least 6 months following the last dose of study medication.]
11. Patient had signed informed consent prior to initiation of any study-specific procedures or treatment.
12. Patient was able to adhere to the study visit schedule and other protocol requirements, including follow-up for OS.

### Exclusion Criteria

1. Patient had received treatment with >2 cytotoxic chemotherapy regimens for CRPC. Chemotherapy in the hormone-sensitive setting did not counted in this assessment provided the last dose was >6 months before study entry. A change in chemotherapy agents due to intolerance after brief exposure might not count in this assessment, pending review with Medical Monitor.
2. Patient had received external-beam radiation or another systemic anticancer therapy within 14 days or 5 half-lives, whichever was shorter, prior to study treatment.
3. Patient had received treatment with an investigational systemic anticancer agent within 14 days prior to study drug administration.
4. Patient had received prior treatment with an anti-PD-1, anti-PD-L1, anti-programmed death-ligand 2 (PD-L2) agent or with an agent directed to another co-inhibitory T-cell receptor (e.g., cytotoxic T-lymphocyte-associated antigen 4 [CTLA-4], OX-40, CD137). or required concomitant treatment with DPP-4 inhibitors (e.g. gliptins).
5. Patient had an additional active malignancy that might confound the assessment of the study endpoints. If the patient had a past cancer history (active malignancy within 2 years prior to study entry) with substantial potential for recurrence, this must be discussed with the sponsor before study entry. Patients with the following concomitant neoplastic diagnoses were eligible: non-melanoma skin cancer and carcinoma in situ (including transitional cell carcinoma, anal carcinoma, and melanoma in situ).
6. Patient had clinically significant cardiovascular disease (e.g., uncontrolled or any New York Heart Association Class 3 or 4 congestive heart failure, uncontrolled angina, history of myocardial infarction, unstable angina or stroke within 6 months prior to study entry, uncontrolled hypertension or clinically significant arrhythmias not controlled by medication).
7. Patient had QT interval corrected for heart rate using Bazett's formula (QTcB)>480 msec at Screening
8. Patient had uncontrolled, clinically significant pulmonary disease (e.g., chronic obstructive pulmonary disease, pulmonary hypertension) that in the opinion of the investigator would put the patient at significant risk for pulmonary complications during the study.
9. Patient had brain or leptomeningeal metastases that were symptomatic and progressive on imaging. Patients with a history of CNS metastases must had received appropriate treatment. CNS imaging was not required prior to study entry unless there was a history of CNS involvement or clinical suspicion of CNS involvement. Imaging of patients with a prior history of CNS metastases should be compared to prior imaging to discern PD.
10. Patient had an active autoimmune disease or Grade ≥3 non-infectious pneumonitis that required systemic treatment in the past 2 years (i.e., with use of disease modifying agents, corticosteroids or immunosuppressive drugs). Replacement therapy (e.g., thyroxine, insulin, or physiologic corticosteroid replacement therapy for adrenal or pituitary insufficiency) or treatment with drugs (e.g., neomercazol, carbimazole, etc.) that function to decrease the generation of thyroid hormone by a hyperfunctioning thyroid gland (e.g., in Graves' disease) was not considered a form of systemic treatment of an autoimmune disease.
11. Patient had a diagnosis of immunodeficiency or was receiving systemic steroid therapy at a prednisone equivalent dose of >10 mg daily for at least 1 week or other form of immunosuppressive therapy within 7 days prior to Cycle 1 Day 1 (C1D1).
12. Patient had uncontrolled intercurrent illness including, but not limited to, uncontrolled infection, suspected or active SARS-CoV-2 (Covid-19) infection disseminated intravascular coagulation, or psychiatric illness/social situations that would limit compliance with study requirements.
13. Patient had known positive status for human immunodeficiency virus active or chronic Hepatitis B or Hepatitis C. Screening was not required.
14. Patient had any medical condition that in the opinion of the investigator placed the patient at an unacceptably high risk for toxicity.
15. Any dysphagia, odynophagia, esophageal dysmotility or stricture, known GI malabsorption syndrome, or intractable diarrhea that might significantly alter the absorption of orally administered study drug.
16. Patients with history of symptomatic orthostatic hypotension within 3 months prior to enrollment. Orthostatic hypotension is defined as a drop in systolic blood pressure (BP) of ≥20 mmHg or diastolic BP of ≥10 mmHg with assumption of an upright posture.

### Study Methodology

Approximately 13 patients with mCRPC were enrolled in the Lead-in Stage of the study.

In the Efficacy Stage, eligible participants were assigned to Cohort A (SCNC) or Cohort B (adenocarcinoma without small cell histology) based on histology/cytology Approximately 15 to 28 patients with SCNC were enrolled in each Cohort. A minimax 2-stage Simon design was applied separately for each cohort. In the first stage, 15 patients were accrued. If there are 2 or fewer composite responses in these 15 patients, accrual to the study arm was halted. Otherwise, 13 additional patients were accrued for a total of 28 patients. A composite response was defined as 1 or more of the following:
Objective response by RECIST 1.1 criteria
CTC conversion from >5/7.5 mL to <5/7.5 mL per Veridex assay by completion of Week 12 of protocol therapy
Greater than 50% PSA decline from baseline by completion of Week 12 of protocol therapy

The tabular Study Schedule of Assessments is found in Table 15.

During the Screening period, patients who signed a consent form were evaluated to ensure they met inclusion and exclusion criteria. Patient demographics, performance status, and disease staging were collected. Vital signs (both sitting and standing blood pressure, heart rate, body temperature, and respiratory rate), physical examination, electrocardiogram (ECG), and clinical laboratory evaluations (complete blood count plus differential, serum chemistry, liver function tests, and urinalysis) were performed at Screening and baseline and were monitored throughout the treatment period. Patients meeting the study entry criteria begun study treatment within 4 weeks of the Screening visit. At the discretion of the investigator, patients with out-of-range clinical laboratory evaluation values at Screening might be retested within the Screening period if the investigator believed that the retest values may be in range and allow inclusion in the study.

Patients undergo tumor assessment, which must include cross-sectional imaging (MRI or CT scanning with IV contrast whenever possible) of the chest/abdomen/pelvis plus whole-body bone scan. Other body sites (e.g., neck) were included as clinically indicated. Tumor assessment was performed at Screening, C4D1 (±7 days), C7D1 (±7 days), C10D1 (±7 days), and Day 1 (±7 days) of every 3rd cycle thereafter.

The same imaging method used to determine index lesion size at baseline must be used to follow lesion size throughout the study. Assessments were conducted throughout the treatment phase as described in Table 15.

Adverse events, clinical laboratory assessments, PSA, and concomitant medications were monitored and recorded throughout the entire study period.

Patients might continue to receive study treatment until the development of radiographic or clinical progression, unacceptable toxicity, another discontinuation criterion was met, or closure of the study by the sponsor; no maximum duration of therapy was set. Treatment beyond progression was allowed.

After discontinuation of study treatment, patients completed an EOT visit within 21 days after their last dose of study drug. Safety Follow-up was conducted 30 days (±7 days) after their last dose of study drug as well as additional subsequent time points if drug-related AEs were not resolved at that time. Patients were also contacted by telephone approximately every 90 days for clinical evidence of PD in settings in which discontinuation of study therapy was for reasons other than PD (tumor measurements as specified in the protocol were not required after the EOT visit), and for assessment of survival status. This extended follow-up for disease status and survival after discontinuation of study treatment continued for up to 12 months after study treatment was discontinued.

Efficacy Assessments

Primary Efficacy Parameter:
The primary efficacy parameter was the composite response rate defined as achieving 1 or more of the following:
   Objective response by RECIST 1.1 criteria
   Circulating tumor cell (CTC) conversion from >5/7.5 mL to <5/7.5 mL per Veridex assay by completion of Week 12 of protocol therapy
   Greater than 50% prostate specific antigen (PSA) decline from baseline by completion of Week 12 of protocol therapy

Secondary Efficacy Parameters:
Radiographic progression-free survival (rPFS)
Prostate specific antigen (PFS)
Overall survival (OS)
Duration of response (DOR)
Level of cytokines previously shown to be modulated by Talabostat in humans.

Exploratory Efficacy Analyses:
To determine the response rate by iRECIST criteria with Talabostat mesylate in combination with Pembrolizumab.
To evaluate the quantitative and qualitative effects of Talabostat mesylate in combination with Pembrolizumab on relevant immune effector cytokines and various immunological effector cells, in blood and, whenever feasible, in tumor tissues.
To explore the predictive value of baseline PD-L1 tumor expression in metastatic tumor tissue and CTCs with subsequent clinical outcomes
To explore the relationship between baseline tumor mRNA immune profiling panel and clinical outcomes.

Safety Assessments

Non-Serious Adverse Events

Investigators assessed for AEs at each visit. All AEs, including observed or volunteered problems, complaints, or symptoms, were recorded on the eCRF. Each AE was to be evaluated for duration, intensity, and causal relationship with the study treatment or other factors.

### Reporting Serious Adverse Events

Any SAE or death occurring during the treatment period and/or within 30 days following the last dose of study medication must be reported to the sponsor or sponsor's representative within 24 hours of first knowledge of the occurrence. If any SAE occurs, study treatment should be interrupted or discontinued at the discretion of the physician investigator.

Adverse Event Follow-Up

Patients were monitored for AEs throughout the treatment period and for a minimum of 30 days after their last dose of Talabostat mesylate.

### Pharmacokinetic Assessments

Sparse pharmacokinetic sampling was performed at the time points described in Table 15 for analysis of concentrations of Talabostat mesylate. Trough samples were taken Pre-dose (−24 hrs) on Day 1 of Cycles 1, 2 and 3. Samples were taken within 30 minutes before the morning dose on Day 14 of Cycles 1, 2, and 3. For Cycle 1 only, a pre-dose sample was taken within 30 minutes before the morning dose of Day 4 (+1 Day). For Cycle 3 only, samples were collected within 30 minutes before the morning dose was administered on Day 14 and at 2 hours (±15 min), 6 hours (±30 min), and if possible, 10-12 hours (optional draw if the patient or site could not accommodate). Additional samples were collected at these approximate times after the Day 14 evening dose: 10-14 hours, 60 hours (±24 hours), 108 hours (±24 hours), and 156 hours. The 156-hour sample was collected within 30 minutes prior to the C4D1 dose. If C4 is not administered, the sample was collected 156 hours after the last dose (±5 hours) (patient diary to be kept to record the number of doses the patient had taken in the cycle). Unscheduled PK assessments might be requested by the Sponsor in order to gather additional information under certain conditions, for example, in the setting of an adverse event. Pharmacokinetic data was analyzed using a population pharmacokinetic approach.

### Pharmacodynamic Assessments

Whole blood samples were collected at the time points described in Table 15 for analysis of relevant immune effector cytokines and various immunological effector cells, in blood and tumor tissues. Unscheduled local cytokine assessments, if possible, might be performed based on clinical judgement or if requested by the Sponsor in order to gather additional information under certain conditions, for example, in the setting of an adverse event.

### Progressive Disease

Worsening signs and symptoms of prostate cancer should be considered by the investigator as disease assessments were being made. PD was assessed as an efficacy outcome in this study and should not be reported as an AE. However, deaths considered solely due to PD occurring within 30 days of the last dose of Talabostat mesylate should be reported as an AE outcome with the AE term reported as “Malignant Neoplasm Progression”.

### Study Completion

The study was considered complete when all patients had been followed to disease progression; were lost to follow-up, death, or withdrawal due to toxicity; patient's request; or investigator's discretion; and had completed all end-of-study treatment procedures.

### Study Medication

Study medication was administered in 21-day cycles. Either Talabostat mesylate or Pembrolizumab may be administered first. However, on Cycle 1 Day 1, it was recommended that Pembrolizumab be administered first and that >1 hour should elapse before the administration of Talabostat mesylate so that it was easier to determine the relatedness of any AEs to study drug

### Talabostat Mesylate Dosage and Administration

Current dosage strengths included 0.05 mg, 0.1 mg and 0.2 mg tablets of Talabostat mesylate for oral administration.

The starting dose regimen of Talabostat mesylate (i.e., the dose regimen in Cohort 1) was 0.4 mg QD on Days 1 to 14 every 21 days. The Talabostat mesylate dose regimen for any patient depended on the cohort in which the patient was enrolled in the Lead-in Stage. Additional dosing schedules were also evaluated during the Lead-in Stage.

To minimize risk of hypotension, patients were advised to maintain adequate hydration while on-treatment, such as drinking at least 2 liters of fluids per day, including fluids with electrolytes. Factors such as strenuous exercise, heat, humidity, fever, gastrointestinal disturbance might increase hydration needs. Administration of at least 1 L of IV fluids is required at Cycle 1 Day 1. It was at the investigator's discretion to provide IV hydration during in-person clinic visits beyond Cycle 1 Day 1.

The patient might be monitored overnight at the discretion of the Investigator. If the overnight monitoring was not associated with any Grade 2 or greater AE or any other SAE criteria, the admission was not be considered an SAE. Longer periods of in-patient monitoring might be implemented at the discretion of the Investigator.

Once the recommended dose for Talabostat mesylate was determined, any patients still tolerating treatment in a lower Talabostat mesylate dose cohort might have their Talabostat doses escalated to the recommended dose regimen with the start of the next cycle at the investigator's discretion.

Talabostat mesylate was administered orally as 0.2 mg, 0.1 mg or 0.05-mg tablet. Patients took the prescribed number of tablets daily on Days 1 to 14 of each cycle, for a total daily dose of 0.4 mg, 0.6 mg, or an intermediate dose. Talabostat mesylate was continued until PD or unacceptable toxicity. Talabostat mesylate should not be taken on an empty stomach.

On days when pharmacodynamic studies were being performed Talabostat mesylate should be administered at the study center, and should be administered at (approximately) the same time of day on each treatment day in the cycle. In cycles in which pharmacodynamics was not evaluated, Talabostat mesylate also should be administered at (approximately) the same time of day on each treatment day in the cycle.

Cycle 1 Dosing

On Cycle 1 Days 1 through 7, patients were instructed to take 0.2 mg BID of Talabostat mesylate. On Cycle 1 Day 8, the patient returned to the clinic for a scheduled visit. During the visit, based on review of the patient's clinical labs, vital signs, and adverse events reporting, the patient's dose of Talabostat mesylate could be escalated to 0.3 mg BID. Patients should not be escalated to 0.3 mg BID of Talabostat mesylate if they experienced any Grade >1 adverse event considered related to Talabostat mesylate or skipped any doses due to hypotension or orthostasis during the first week of treatment.

During the dosing period of Cycle 1, the patient returned to the clinic on Day 2 and Day 4 (+1 Day). During these visits, blood draws were taken for pharmacokinetic and pharmacodynamic parameters, adverse events were assessed, and vital signs were measured prior to dosing. Patients were instructed not to take their morning dose of Talabostat mesylate until their vital signs had been taken and reviewed by the study physician.

Daily during the dosing period of Cycle 1, the patient performed at home blood pressure monitoring at least twice daily, once in the morning and once in the late afternoon/evening. The patient was provided with a log to record blood pressure measurements and was asked to bring the log with them to every clinic visit during Cycle 1. The study team contacted the patient daily during treatment period of Cycle 1, through Day 14, to remind the patient of oral hydration guidelines, review side effects, and to review the patient's self-administered blood pressure measurements. The patient must take their blood pressure prior to dosing in the morning and evening. The patient was instructed that they must not take their dose if their blood pressure was below 100 mmHg systolic or 50 mmHg diastolic. The patient must report any values below this to study physician immediately. The study physician provided the patient further instruction as needed.

If the patient did not met criteria to increase their dose to 0.3 mg BID during Cycle 1, they might continue on treatment at 0.2 mg BID until the investigator decided to escalate the patient's dose to 0.3 mg BID. During the first week of treatment at 0.3 mg BID, the patient performed at home blood pressure monitoring at least twice daily, once in the morning and once in the late afternoon/evening, prior to dosing. The patient recorded blood pressure measurements on a log and was asked to bring the log with them to every clinic visit during the first cycle that their dose was escalated to 0.3 mg BID. The patient was instructed that they must not take their dose if their blood pressure was below 100 mmHg systolic or 50 mmHg diastolic. The patient must report any values below this to study physician immediately. The study physician provided the patient further instruction as needed. The study team contacted the patient daily during the first week of treatment at the 0.3 mg BID dose level to remind the patient of oral hydration guidelines, review side effects, and to review the patient's self-administered blood pressure measurements.

Dose Adjustments of Talabostat Mesylate Secondary to Toxicity: Talabostat mesylate dose modifications within a treatment cycle were not permitted in Cycle 1 in the absence of DLT. In Cycle ≥2, dose modifications within a treatment cycle was at the discretion of the investigator. Doses were taken at approximately the same time every day. Doses held because of AEs should not be made up on subsequent days within or following a cycle. A dose that was missed for reasons other than an AE (i.e., the patient forgets to take a dose) might be taken at least 6 hours prior to the next planned dose; otherwise, the missed dose might be administered on days subsequent to scheduled doses. Any additional adjustments should be discussed with the Medical Monitor or designee. If a dose was vomited within approximately 10 minutes of dosing, the patient may be re-dosed. If the patient vomited>10 minutes after dosing, no further attempts at dosing that particular dose should take place; dosing should resume with the next dose. Under no circumstances should missed doses be made-up on a day when the patient was already taking a planned dose (i.e., no “doubling-up” to account for missed doses).

If an SAE thought to be related to Talabostat Mesylate occurred during the treatment period, dosing of Talabostat Mesylate was interrupted in that patient until the SAE resolved. If the investigator wished to continue the patient on Talabostat Mesylate, the medical monitor was contacted to discuss continuing Talabostat Mesylate at the same or reduced dose.

The most frequently observed AEs that appeared to be characteristic of Talabostat Mesylate were edema/peripheral swelling, hypotension, dizziness, and hypovolemia. These events, including edema, tend to be manageable and reversible and usually resolved following a drug hold. Talabostat Mesylate should be held for Grade 2 or higher episodes of such events, until resolution of these AEs. Talabostat Mesylate could be restarted at full dose after resolution of these AEs, including edema. For other Grade 2 or higher AEs deemed related to Talabostat Mesylate, or for edema that did not respond to drug hold, the dose of Talabostat Mesylate could be reduced by 0.2 mg decrements of the total daily dose at the discretion of the investigator.

Discontinuation of Talabostat Mesylate should occur for any life-threatening AE, or for Grade 2 or higher treatment-related AEs that did not respond to dose reduction to a 0.2 mg total daily dose. If Talabostat Mesylate was discontinued due to an AE, all termination from treatment procedures and assessments must be performed.

Monitoring of Patient Compliance with Talabostat Mesylate Study Medication

Patients must be at least 70% compliant with taking Talabostat mesylate in Cycles 1 and 2 in order to be included in the per-protocol efficacy analyses.

Talabostat Mesylate Description and Storage

Talabostat mesylate was supplied as 0.2-mg tablets, 0.1-mg tablets and 0.05-mg tablets in high-density polyethylene bottles with desiccant and child-resistant caps. The 0.2-mg strength was supplied as 30 tablets per bottle. The 0.1-mg tablets were supplied as 90 tablets per bottle. The 0.05-mg strength was supplied as 90 tablets per bottle.

Supplies of Talabostat mesylate was appropriately labelled for clinical trial material. Talabostat mesylate should be stored under refrigerated conditions between 2° C. to 8° C. (36° F. to 46° F.) until use. During the period of use, meaning once dispensed for patient, Talabostat mesylate tablets might be stored in their original container for up to 30 days at room temperature, 15°-25° C. (59°-77° F.).

### Pembrolizumab (PEMBRO):

The Pembrolizumab dose was 200 mg, administered as an IV infusion over 30 minutes on Day 1 of each 21-day cycle. Premedication, according to standard local practices, was permitted

### Data Analysis and Statistical Considerations

Analysis Populations

The modified intent-to-treat (mITT) analysis population consisted of patients enrolled in the Efficacy Stage who met the eligibility criteria and received at least 1 dose of Talabostat mesylate and Pembrolizumab. The response evaluable patient population consisted of patients enrolled in the Efficacy Stage who completed at least 2 cycles of treatment with combined Talabostat mesylate and Pembrolizumab, with at least 1 post-baseline response assessment made by the investigator(s).

The safety population consisted of all patients who received any dose of Talabostat mesylate/Pembrolizumab, either during the Lead-in or Efficacy Stages of the study.

The pharmacodynamic analysis population consisted of all patients who received any dose of Talabostat mesylate/Pembrolizumab and had cytokine levels measured at least once.

The pharmacokinetics population include all patients who received at least 1 dose of Talabostat mesylate and had at least 1 quantifiable Talabostat mesylate plasma concentration at a scheduled pharmacokinetics time point postdose.

Analysis of Demographics and Baseline Characteristics

Demographics and baseline disease characteristics were summarized and listed for the safety analysis population. If the number of patients in the safety analysis population differed substantively from the number of patients in the response evaluable or mITT analysis population, demographics and baseline characteristics for these analysis populations might be presented.

Efficacy Data Analysis: The primary and secondary efficacy parameters were defined herein.

Stage 1 Analysis: The Stage 1 analysis was performed for Cohort A and Cohort B, separately. In each cohort, when 15 patients had completed approximately 6 cycles of treatment and had 2 post-baseline tumor assessments and PSA or CTC measurements, the number of patients who met the composite response criteria of achieving 1 or more the following: 1) objective response by RECIST 1.1 criteria, 2)>50% decline from baseline in serum PSA by Week 12 of treatment, or 3) CTC conversion from >5/7.5 mL to <5/7.5 mL per Veridex assay by completion of Week 12 of protocol therapy were evaluated.

Using minimax 2-stage Simon design, if 2 or fewer out of 15 stage 1 patients met at least 1 of the composite response criteria, enrollment was stopped. This indicated that data to date was consistent with the null hypothesis that the composite response rate was 15% or less, thereby rejecting the alternative hypothesis that the composite response rate was 35%. If 3 or more out of 15 patients met at least 1 of the composite response criteria, 13 more patients were enrolled and treated to proceed to stage 2, for a total of 28 patients in both stages

Stage 2 Analysis: In each Cohort enrolling in Stage 2, when the additional 13 patients in stage 2 enrolled and treated had completed approximately 6 cycles of treatment, 2 post-baseline assessments of tumor, and PSA and CTCs measurements, the number of patients who met at least 1 of the 3 criteria for the composite endpoint was evaluated. If the total number of patients who met the composite endpoint in 28 patients in both stages was 7 or less, then data were consistent with the null hypothesis of composite endpoint rate of 15% or lower with nominal 0.05 1-sided significance level. If the number of patients who met the composite endpoint was 8 or more, then the data were consistent with the composite endpoint rate of at least 35%. The composite endpoint rate across 2 stages and its exact 95% confidence interval (CI) was calculated as if data were collected in a single stage. This approach that ignored the sequential statistical testing might lead to biased point estimate of the composite endpoint rate and the CI might not provide the stated coverage probability, but was generally accepted when the event rate was relatively small.

Sensitivity Analysis: The composite endpoint rate were also be calculated for the mITT analysis population. Patients in the mITT population with missing composite endpoint were considered non-responders (e.g., not 1 of the 3 criteria for composite endpoint was met).

Analysis of Secondary Parameter(s): Time-to-Event Response. The distribution of time-to-event response including rPFS, PSA, PFS, DOR, and OS was estimated by Kaplan-Meier methodology. The medians of these time-to-event efficacy responses, if available, and their 2-sided 95% CI, was reported. In addition, the proportions of patients with events at selected time points, together with their 2-sided 95% CI was presented. The calculations were performed based on fixed sample, single stage design.

The primary analysis was performed using the mITT analysis population. As a supplement, time-to-event analysis was performed using the response evaluable analysis population.

Analyses of the duration of overall objective response was performed for all patients in the mITT analysis population who achieved confirmed PR or CR. The number of CR and PR patients might be small, and thereby limited use of the Kaplan-Meier method to provide reliable information. In this case, descriptive statistics or listings were provided.

After discontinuing the study medication, patients might be treated with additional therapy. Data collected after patients were treated with additional therapy was not used to evaluate the duration of objective response.

Analysis of Secondary Endpoints: Cytokine Levels. The analysis of the secondary efficacy endpoints including cytokine levels was reported for the PD analysis population. Descriptive statistics for levels of cytokines previously shown to be modulated by Talabostat mesylate in human was reported.

Analysis of Exploratory Endpoints: The exploratory endpoints were analyzed using the response evaluable analysis population.

The proportion of patients who met the iRECIST criteria was presented.

At the minimum, the proportion of who experienced clinical benefit (PR, CR) and OS was reported.

Whenever feasible, cross tabulation of clinical outcomes by presence/absence of relevant immune effector cytokines and various immunological effector cells in blood or in tumor tissues was presented.

The baseline PD-L1 tumor expression in metastatic tumor tissue and CTCs was cross-tabulated with subsequent clinical outcomes. The baseline tumor mRNA immune profiling panel was cross-tabulated with clinical outcomes with regards to response and safety.

Analysis of Treatment Exposure: Descriptive summary statistics was provided for total number of cycles, doses, average dose administered, and duration of treatment.

Analysis of Patient Study Disposition: The number of patients enrolled who met the criteria for the mITT analysis, safety, response evaluable, and pharmacodynamic populations was reported.

The number of patients discontinuing the study over time was summarized for the safety population.

Statistical Power and Sample Size Considerations: For each cohort, a total sample size of 28 patients, 15 patients at stage 1 and 13 patients at stage 2, was treated with combined Talabostat mesylate and Pembrolizumab in order to detect with 80% power an alternative hypothesis percentage of patients who met the composite endpoint of 35% versus the null hypothesis that the percentage of 15%, with early stopping for futility at stage 1, in a 1-sided test with 0.05 significance level (actual value is 0.0461). Two or fewer patients who met the composite endpoint at stage 1 triggered early stopping. Seven or fewer patients among 28 patients in both stages lead to rejecting the alternative hypothesis that the composite endpoint rate was at least 35%.

Safety Analyses: All patients in the safety population included in the final summaries and listings of safety data, separately for the lead-in patients grouped into cohorts. Summaries of AEs and other safety parameters was provided as appropriate. Emphasis in the analysis of AEs was placed on those that were treatment-emergent through 30 days after last dose of Talabostat mesylate/Pembrolizumab. Frequencies of patients experiencing at least 1 AE were displayed by body system and preferred term according to Medical Dictionary for Regulatory Activities (MedDRA) terminology. Intensity (severity) of the AE was graded according to NCI CTCAE.

Replacement of Patients: In the Lead-In Stage, patients who were assigned a patient number and who discontinued treatment before completion of the DLT period without experiencing a DLT were replaced for DLT assessment purposes.

In the Efficacy Stage, patients who were assigned a patient number and who discontinued treatment for reasons other than Adverse Event or Progressive Disease before receiving at least 2 cycles of Talabostat mesylate were replaced and excluded from the response evaluable patient population. The reasons for treatment discontinuation and study discontinuation was also be collected for replaced patients.

Summary and Results:

Interim data: A total of 13 patients with mCRPC were treated with Talabostat mesylate and pembrolizumab in the Phase 1b-Lead in stage of the current clinical study. Seven patients having adenocarcinoma and 6 SC/t-NEPC including mixed on most recent pathology. Prior treatments included androgen deprivation therapy (ADT) (n=10), 2^{nd} Generation androgen signaling inhibitors (ASI) (n=9), chemotherapy (n=11), radiotherapy (n=11) (see Table 4).

Initially, 3 patients were treated with Talabostat mesylate at the initial dose level of 0.4 mg QD for 14 days of a 21 day cycle plus the standard dose of pembrolizumab 200 mg IV on day 1 of each cycle. Two patients discontinued for disease progression: one after 6 cycle and one at 16 cycles respectively. One of the patient discontinued at cycle 17 for an adverse event unrelated to study therapy. No treatment related (TR) DLTs or TR-SAEs were reported (Table 5). Grade ≥3 TRAEs were limited to Grade 3 anemia in 2 patients.

Next, among 3 patients treated at the Talabostat mesylate dose level of 0.6 mg QD, 1 patient had a dose-limiting toxicity of Grade 3 syncope (C1D6) and 1 patient had an SAE of Grade 5 acidosis with cardiac arrest (C3) (Table 5). The 0.6 mg dose level was then expanded (n=7) using a split dose strategy in order to improve tolerability while maintaining the total daily dose associated with objective responses in prior studies. No DLTs or TR-SAEs was reported during the DLT period among patients receiving the 0.6 mg split dose (Table 5). A dose-dependent increase in on-target clinical effects was observed. In the 0.4 mg QD cohort no patients experienced TR on-target clinical effects, whereas, in the 0.6 mg QD cohort, all patients had events consistent with cytokine release, 3/3 patients had hypotension (including 1 grade 3 syncope (DLT) and 2 patients each had dizziness and LE edema (Table 8). The split dose strategy resulted in improved the tolerability with fewer patient experiencing events consistent with cytokine release, with nearly all of the events occurring at low grades (3/7 patients had fatigue [grade 3 in a patient with fatigue present from baseline], and 1 patient each had low grade hypotension, dyspnea, chills, myalgia) while maintaining the TDD (0.6 mg) previously associated with objective response.

During the course of treatment patients with RECIST 1.1 defined measurable disease are monitored for RECIST 1.1 response and patients with pre-treatment PSA >0 ng/ml are monitored for PSA response. Based on preliminary data, 4 of 8 RECIST-evaluable patients achieved a RECIST 1.1 best response of stable disease, with no CR/PR reported (Table 6). One of 10 PSA response-evaluable patients achieved a PSA response defined as a 50% reduction (Table 6). The subject disposition is included in Table 7. Results from the Phase 1b safety assessment of Talabostat mesylate support the use of a split dose (am and pm) totaling 0.6 mg per day as the recommended dose when used in combination with pembrolizumab.

In this study:
On-target side effects consistent with cytokine activation were seen at the highest daily dose tested.
Splitting the daily dose (0.6 mg) was associated with improved tolerability as evidenced by no reported DLTs and lower rates of other key side effects such as hypotension and peripheral edema.
Long-term safety was demonstrated at the lower dose level, while data accumulates from patients continuing with the split dose regimen at 0.6 mg daily.
Consistent dose and time dependent increases in IL-18 levels observed in 0.6 mg split dose, maximal changes noted after 14 days of continuous dosing. Minimal, short-duration changes noted in cytokines often associated with AEs.
Preliminary evaluation of anti-tumor activity from the Safety Lead-in portion of the study includes 1 patient with a PSA response and RECIST 1.1 Partial Response and three patients with stable disease per RECIST 1.1 including 1 patient still on treatment.

The Phase 2 efficacy portion of this study assessed the anti-tumor activity of the combination in patients with mCRPC of either SC/t-NEPC or adenocarcinoma phenotype of the combination in a setting where checkpoint inhibitor monotherapies demonstrated limited clinical benefit.

Details of one confirmed RECIST 1.1 and PSA responder in Phase 1B—

Phase 1b PID-112-207: RECIST 1.1 and PSA response:

Patient Demographics and History: 73 years
73 years old, diagnosed in 2006 with Adenocarcinoma
Other medical history: coronary artery disease, hypertension, MI (2003), limb edema,
Sites of disease at study entry: multiple lymph nodes and bone lesions
Baseline PSA 163.1 ng/ml
Prior Systemic Therapy included: Bicalutamide; Abiraterone+Prednisone; Enzalutamide; Sipuleucel; Docetaxel/Cabazitaxel.
Tempus molecular testing cfDNA peripheral blood-MSI-high not detected; no reportable treatment options found. On-treatment disease status: Duration of Talabostat Mesylate+Pembrolizumab: 16+ cycles. PSA₅₀ response by 6 weeks, max reduction 84%. RECIST 1.1 Status: Partial Response by 12 weeks: 35% reduction in target lesions, disappearance of ⅖ non-target lymph nodes and non PD/non-CR bone disease.

TABLE 4
Phase 1b-Safety Lead-in stage-Patient disease history

| | | No. of Subjects | | | |
|---|---|---|---|---|---|
| | | Talabostat | Talabostat | Talabostat | |
| | | mesylate (0.4 | mesy late (0.6 | mesylate (0.6 | |
| | | mg) + | mg) + | mg split dose) | |
| | | Pembrolizumab | Pembrolizumab | Pembrolizumab | All |
| Baseline Characteristics | | N = 3 | N = 3 | N = 7 | (N = 13) |
| Age | Mean | 70 | 71 | 73 | 72 |
| | (range), years | (61-75) | (62-86) | (62-84) | (61-86) |
| ECOG PS | PS 0/1/2 (n) | 2/1/0 | 2/1/0 | 1/6/0 | 5/8/0 |
| 0/1/2 (n) | | | | | |
| Most Recent | Adenocarcinoma | 3 | 2 | 2 | 7 |
| Histopathology | SC/t-NEPC | — | 1 | 5 | 6 |
| | including Mixed | | | | |
| Prior Systemic | Any ADT | 2 | 3 | 5 | 10 |
| Therapies** | therapy | | | | |
| | 1^{st} generation | 1 | 2 | 3 | 6 |
| | ADT therapy | | | | |
| | 2nd Generation | 2 | 2 | 5 | 9 |
| | ASI | | | | |
| | Chemotherapy | 3 | 1 | 7 | 11 |
| | Radiotherapy | 2 | 3 | 6 | 11 |

ADT—Androgen deprivation therapy; ASI—Androgen signalling inhibitor; *For Patients with SC/t-NEPC mean age is 72 (range 62-84)

TABLE 5
Phase 1b-Safety Lead-in stage-Safety data summary

| | No. of Patients | | |
|---|---|---|---|
| | Talabostat | Talabostat | Talabostat |
| | mesylate (0.4 | mesylate | mesylate (0.6 |
| | mg) + | (0.6 mg) + | mg split dose) + |
| | Pembrolizumab | Pembrolizumab | Pembrolizumab |
| Safety | N=3 | N=3 | N=7 |
| Any DLT in cycle 1 | 0 | 1 | 0 |
| Any SAE | 2 | 1 | 4 |
| Any treatment | 0 | 1 | 1 |
| related SAE* | | | |
| Treatment related | 0 | 1 | 1 |
| AE leading to | | | |
| Discontinuation | | | |
| Any Talabostat | 3 | 3 | 5 |
| mesylate | | | |
| treatment related AE* | | | |
| Any Pembrolizumab | 2 | 2 | 5 |
| treatment | | | |
| related AE* | | | |
| Any Talabostat | | | |
| mesylate or | | | |
| Pembrolizumab | | | |
| treatment | | | |
| related Grade | | | |
| 3 or 4 AE* | | | |
| Non-hematologic | — | 2# | 3\hat{ } |
| Hematologic | 3** | 1## | 1\hat{ }\hat{ } |

*Includes related and possibly-related events
**2 patients: Grade 3 anemia; 1 pt Grade 4 thrombocytopenia
#1 patient each: Grade 3 syncope (DLT C1D6) and Grade 5 acidosis (C3)
##1 patient with Grade 3 Hgb and WBC increase
\hat{ }1 patient: Grade 3 Fatigue, fatigue present at baseline, 1pt Grade 3 pneumonitis, SOB, fatigue, hyponatremia, 1pt Grade 3 lactic acid increase
\hat{ }\hat{ }1Patients: Grade 3 Anemia

TABLE 6
Phase 1b-Preliminary efficacy

| | | | |
|---|---|---|---|
| | Talabostat | Talabostat | Talabostat |
| | mesylate (0.4 | mesylate (0.6 | mesylate (0.6 |
| | mg qd) + | mg qd) + | mg split dose) + |
| | Pembrolizumab | Pembrolizumab | Pembrolizumab |
| Responses | N = 3 | N = 3 | N = 7 |

RECIST 1.1 Evaluable^{t}
Response

| | | | |
|---|---|---|---|
| SD | 2 | 1 | 1 |
| Non-CR/Non-PD | 1 | — | 1 |
| PD | — | 1 | 2 |
| NE | — | 1* | 3** |
| PSA Evaluable | 3 | 2 | 5 |
| PSA response^{tt} | 0 | 0 | 1 |

^{t}Measurable disease at baseline
^{tt}≥50% decline by week 12
*early death or DC without follow-up disease assessment,
**too early for both patients

TABLE 7
Phase 1b-Safety Lead-in Stage-Subject disposition

| | | | | | | | | | | | | | | | | | | |
|---|---|---|---|---|---|---|---|---|---|---|---|---|---|---|---|---|---|---|
| Patient | C1 | C2 | C3 | C4 | C5 | C6 | C7 | C8 | C9 | C10 | C11 | C12 | C13 | C14 | C15 | C16 | C17 | Off Tx |
| # | D1 | D1 | D1 | D1 | D1 | D1 | D1 | D1 | D1 | D1 | D1 | D1 | D1 | D1 | D1 | D1 | D1 | Reason |

Cohort #1: Talabostat mesylate 0.4 mg qd + Pembrolizumab (N = 3)

| | | | | | | | | | | | | | | | | | | |
|---|---|---|---|---|---|---|---|---|---|---|---|---|---|---|---|---|---|---|
| 103-101 | X | X | X | X | X | X | DC | | | | | | | | | | | Progression |
| 103-102 | X | X | X | X | X | X | X | X | X | X | X | X | X | X | X | DC | | Progression |
| 103-103 | X | X | X | X | X | X | X | X | X | X | X | X | X | X | X | X | DC | Unrelated |
| | | | | | | | | | | | | | | | | | | Adverse |
| | | | | | | | | | | | | | | | | | | event |

Cohort #2a: Talabostat mesylate 0.6 mg qd + Pembrolizumab (N = 3)

| | | | | | | | |
|---|---|---|---|---|---|---|---|
| 106-201 | X | X | X | DC | | | Adverse |
| | | | | | | | event |
| 101-202 | X | X | X | X | X | DC | Investigator |
| | | | | | | | Decision |
| 101-203* | X | X | X | X | X | DC | Progression |

Cohort #2b: Talabostat mesylate 0.6 mg TDD split dose + Pembrolizumab (N = 7)

| | | | | | | | | | | | | |
|---|---|---|---|---|---|---|---|---|---|---|---|---|
| 101-204 | X | DC | | | | | | | | | | WC |
| 112-205 | X | X | DC | | | | | | | | | Progression |
| 112-207 | X | X | X | X | X | X | X | X | X | X | X | Continuing |
| 128-208 | X | X | DC | | | | | | | | | Progression |
| 115-209 | X | X | DC | | | | | | | | | AE |
| 106-210 | X | X | X | X | X | X | X | X | X | DC | | Progression |
| 101-211 | X | X | DC | | | | | | | | | Progression |

C = Cycle defined as 21 days: Talabostat mesylate PO days 1-14 + Pembrolizumab 200 mg IV day 1
DC = Discontinued treatment
AE: Adverse event
WC: withdrawal consent
Inv Dec: Investigator decision
Patient*101-203 after Cycle 1 was on a reduced dose of 0.2 mg BID due to DLT. Patient discontinued due to disease progression
Patient 103-103-discontinued due to unrelated AE
Patient 106-201-discontinued due to related AE (Grade 5 acidosis)
Patient101-202 discontinued due to clinical disease progression and investigator decision
Patient 101-204 discontinued due to withdrawal of consent (progressive disease. Refused further treatment). Not evaluable for DLT.
Patient 112-205 discontinued due to Investigator decision (clinical disease progression)
Patient 115-209 discontinued due to Related AE (pembro-induced pneumonitis)

TABLE 8
Talabostat on-target side effects consistent with cytokine activation were seen at the
highest doses

| | Talabostat mesylate (0.4 mg | | | | Talabostat mesylate (0.6 mg | | | | Talabostat mesylate (0.6 | | | |
|---|---|---|---|---|---|---|---|---|---|---|---|---|
| Preferred | qd) + Pembrolizumab | | | | qd) + Pembrolizumab | | | | mg split dose) + | | | |
| term | N = 3 | | | | N = 3 | | | | Pembrolizumab N = 7 | | | |
| | Grade n | | | | | | | | | | | |
| Talabostat | Any | 1 | 2 | 3 | Any | 1 | 2 | 3 | Any | 1 | 2 | 3 |
| related | | | | | | | | | | | | |
| events# | | | | | | | | | | | | |
| Hypotension* | — | — | — | — | 3 | 2 | 1 | — | 1 | 1 | — | — |
| Dizziness | — | — | — | — | 2 | 2 | — | — | — | — | — | — |
| Headache | — | — | — | — | 1 | 1 | — | — | — | — | — | — |
| Syncope | — | — | — | — | 1 | — | — | 1 | — | — | — | — |
| Dyspnea | — | — | — | — | 1 | 1 | — | — | 1 | — | 1 | — |
| Chills | — | — | — | — | — | — | — | — | 1 | — | 1 | — |
| Edema | — | — | — | — | 2 | 1 | 1 | — | — | — | — | — |
| Fatigue | — | — | — | — | 1 | — | 1 | — | 3 | 2 | — | 1^{†} |
| Myalgia | — | — | — | — | 1 | 1 | — | — | 1 | 1 | — | — |

#Relatedness as assessed by investigator
*Includes orthostatic hypotension
^{†}fatigue present at baseline
Other Talabostat related adverse events Grade ≥3 (1 acidosis) or low grade in ≥2 patients (arthralgia, hematologic, decrease appetite)

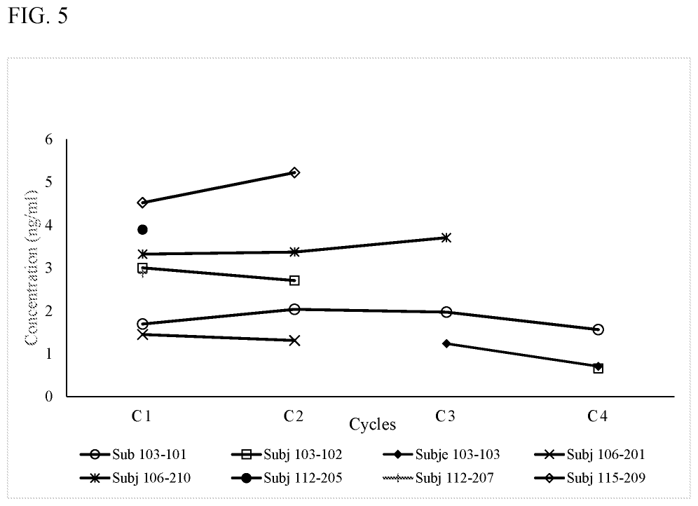

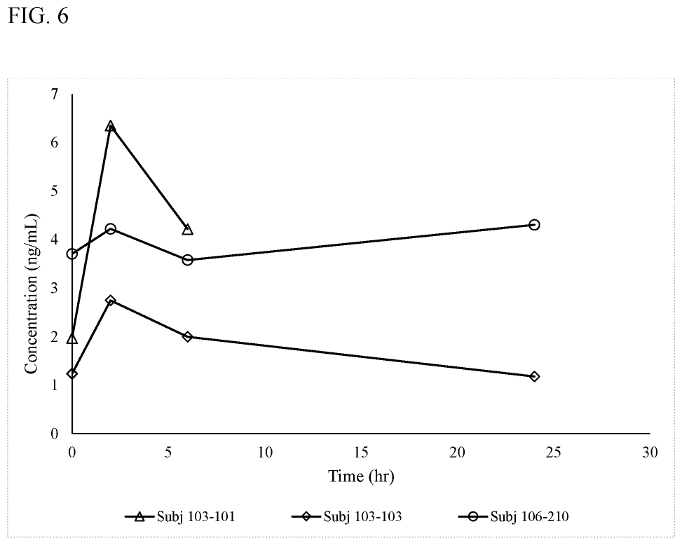

Pharmacokinetic: Talabostat mesylate was quantifiable in plasma and shown in FIG. 5 and FIG. 6. The dosing schedule given to the patients was as follows:
0.4 mg QD Talabostat mesylate+Pembrolizumab was given to the subject no. 103-101/103-102/103-103,
0.6 mg QD Talabostat mesylate+Pembrolizumab was given to the subject no. 106-201,
0.6 mg TDD (split) Talabostat mesylate+Pembrolizumab was given to the subject no. 106-210/112-205/112-207/115-209.

The sample collection was done at the following time points and further defined in Table 9:
C1, C2, C3: at Day 14 pre-dose
C3, Day 14: 2, 6, 12, 24-96 hr post-dose
C4: Day 1 pre-dose

TABLE 9
Patient Pharmacokinetic sampling record

| | Time | Subject No. | | | | | | | |
|---|---|---|---|---|---|---|---|---|---|
| Time point | (cycle) | 103-101 | 103-102 | 103-103 | 106-201 | 106-210 | 112-205 | 112-207 | 115-209 |
| C1 Day 14 | C1 | ✓ | ✓ | x | ✓ | ✓ | ✓ | ✓ | ✓ |
| Pre | | | | | | | | | |
| C2 Day 14 | C2 | ✓ | ✓ | x | ✓ | ✓ | x | x | ✓ |
| Pre | | | | | | | | | |
| C3 Day 14 | C3 | ✓ | x | ✓ | x | ✓ | x | x | x |
| Pre | | | | | | | | | |
| C4Day 1 pre | C4 | ✓ | ✓ | ✓ | x | BLQ | x | x | x |
| C3 Day 14 | 0 | ✓ | x | ✓ | x | ✓ | x | x | x |
| Pre | | | | | | | | | |
| C3 Day 14 | 2 | ✓ | x | ✓ | x | ✓ | x | x | x |
| 2 hr | | | | | | | | | |
| C3 Day 14 | 6 | ✓ | x | ✓ | x | ✓ | x | x | x |
| 6 hr | | | | | | | | | |
| C3 Day 14 | 12 | x | x | x | x | x | x | x | x |
| 12 hr | | | | | | | | | |
| C3 Day 14 | 24 | x | x | ✓ | x | ✓ | x | x | x |
| 24-96 hr | | | | | | | | | |

### Cytokine Release:

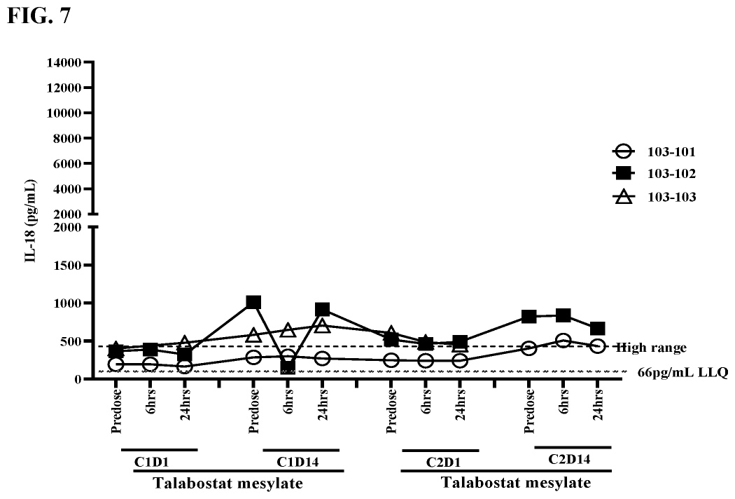

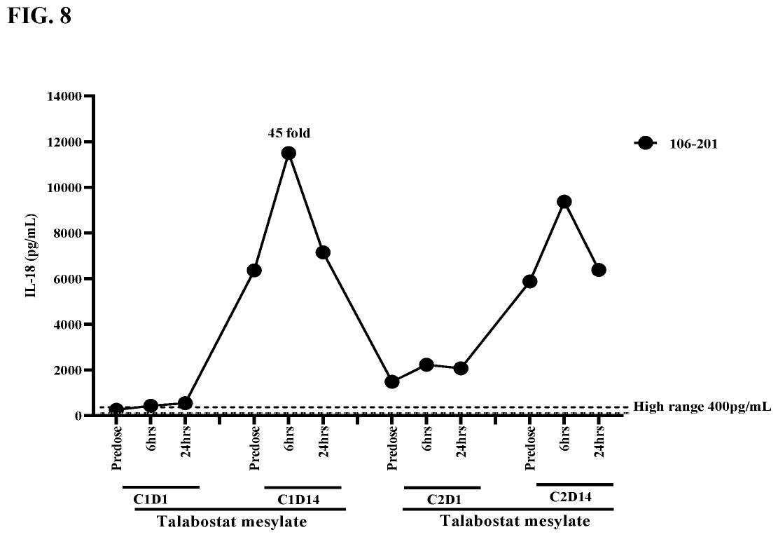

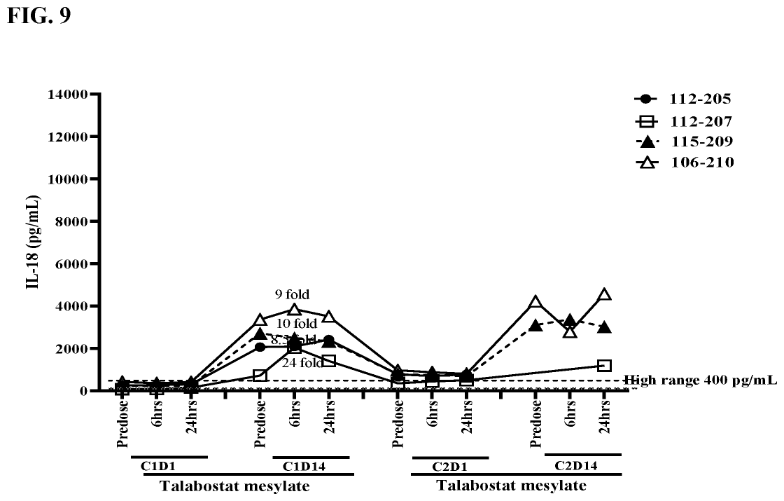

IL-18 Changes post-dose: Consistent dose and time dependent increases in IL-18 levels in the serum were observed with all the dose cohorts (Table 10 and FIGS. 7-9). The 0.6 mg split dose (0.3 mg BID-Talabostat mesylate) plus Pembrolizumab showed consistent increases in IL-18 on day 14 post dose (8-24-fold). Patient #106-201 (0.6 mg QD+pembrolizumab) showed maximum IL-18 levels (45-fold). Consistent dose and time dependent increases in IL-18 levels observed in 0.6 mg split dose, maximal changes noted on day 14.

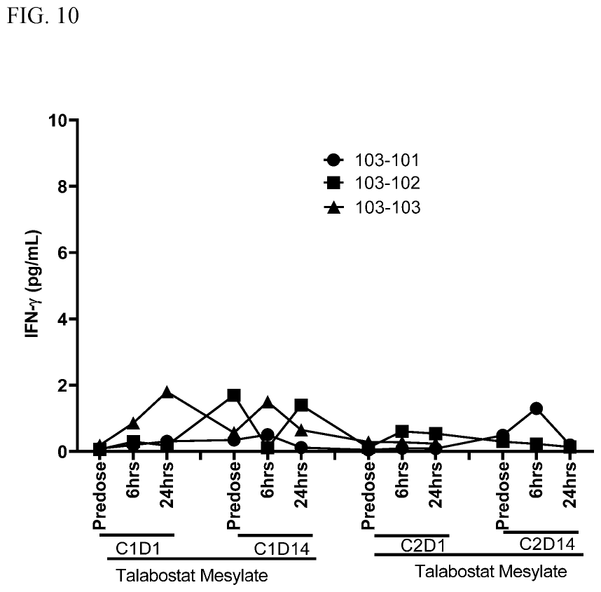

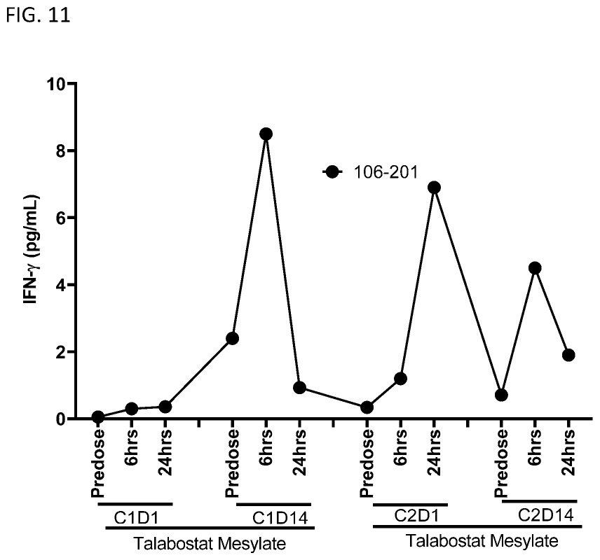

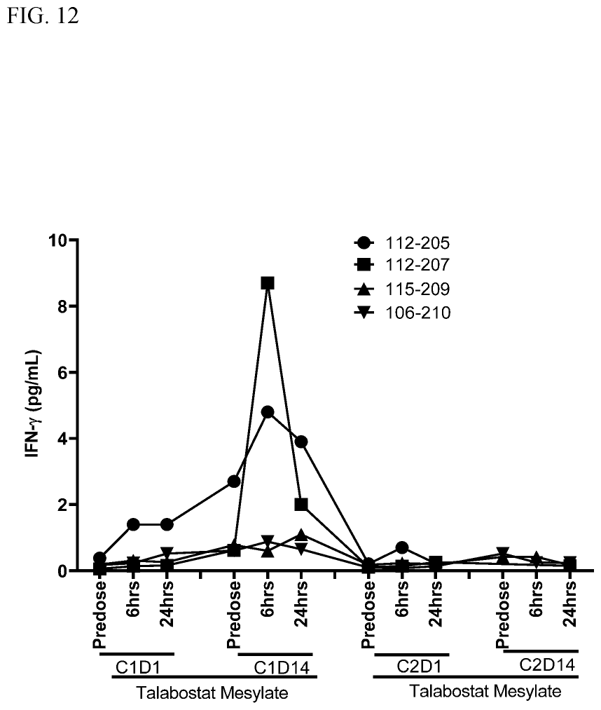

IFN-γ Changes post-dose: IFN-γ is produced by activated T-cells and NK cells; high levels are associated with effective host defense and anti-tumor immunity. Time dependent increase in IFN-γ levels was observed in 0.6 mg qd and split dose cohorts (FIGS. 10-12).

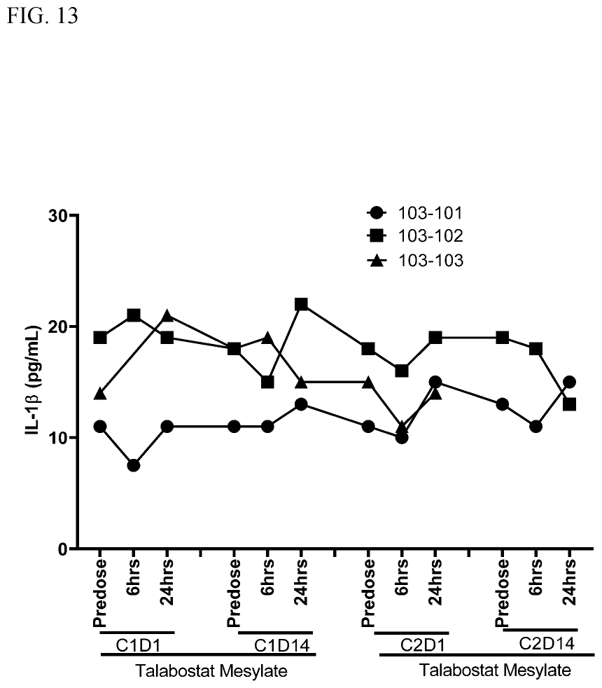

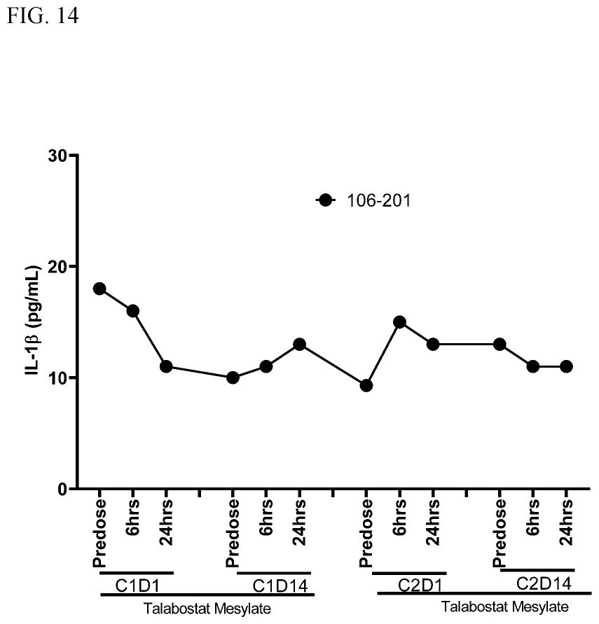

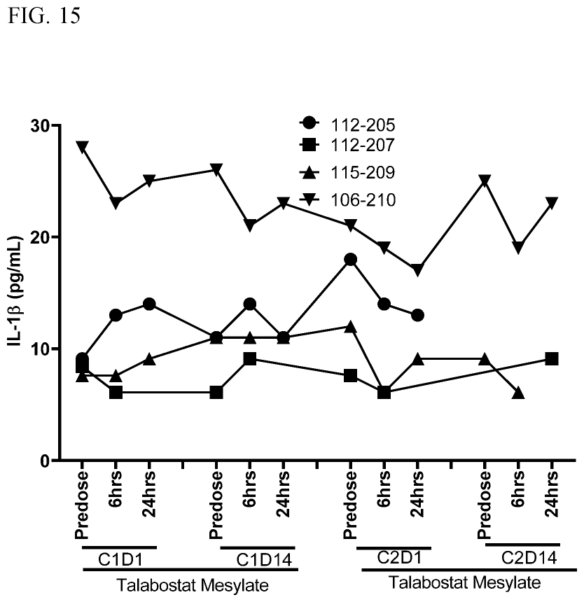

IL-1β Changes post-dose: IL-1β is a pro-inflammatory cytokine produced by innate immune cells. IL-1β is associated with safety and is pro-tumorigenic. IL-1β levels did not change much in 0.4 mg qd+pembrolizumab dose group, neither in 0.6 mg qd and split dose cohorts during dosing (FIGS. 13-15)

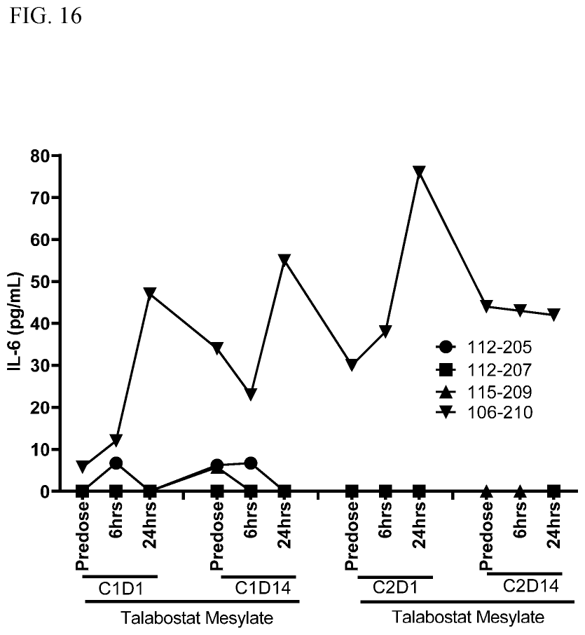

IL-6 (0.6 mg split dose Talabostat mesylate+Pembrolizumab): IL-6 is a pleiotropic cytokine. IL-6 helps tumor cell proliferation. Increased IL-6 associated with cytokine release syndrome. In the majority of patients, any increase in IL-6 quickly returned to baseline (FIG. 16).

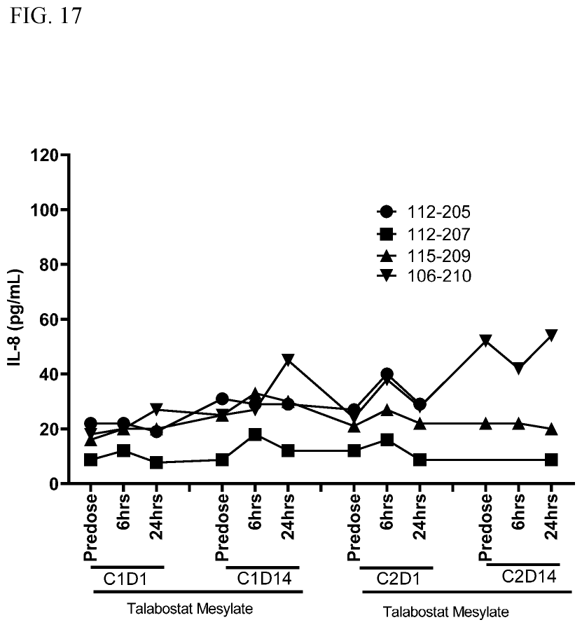

IL-8 (0.6 mg split dose Talabostat mesylate+Pembrolizumab): IL-8, neutrophil chemotactic factor, induces chemotaxis in neutrophils and granulocytes. Increased IL-8 associated with safety adverse events. Post-treatment IL-8 levels remained similar to baseline levels (FIG. 17)

TABLE 10
shows the IL-18 changes (pg/ml) in the serum post dosing of Talabostat and
Pembrolizumab

| | Serum levels (pg/ml) after dose | | | | | | | | | |
|---|---|---|---|---|---|---|---|---|---|---|
| | | | | | 0.6 mg QD | | | | | |
| | | | | | (Talabostat | | | | | |
| | | | | | mesylate) + | | | | | |
| | 0.4 mg QD (Talabostat mesylate) + | | | | Pembrolizumab | 0.6 mg split dose (0.3 mg BID-Talabostat | | | | |
| Dose | Pembrolizumab (n = 3) | | | | (n = 1) | mesylate) + Pembrolizumab (n = 4) | | | | |
| Time | 103-101 | 103-102 | 103-103 | Mean ± SD | 106-201 | 112-205 | 112-207 | 115-209 | 106-210 | Mean ± SD |
| Predose | 194 | 366 | 400 | 320 ± 110.43 | 255 | 244 | 84 | 244 | 426 | 249.5 ± 139.76 |
| 6 hr | 194 | 388 | QNS | 291 ± 137.17 | 432 | 270 | 97 | 230 | 361 | 239.5 ± 109.67 |
| 24 hr | 164 | 322 | 478 | 321.33 ± 157.00 | 542 | 309 | 151 | 250 | 413 | 280.75 ± 109.64 |
| Predose | 285 | 1010 | 582 | 625.66 ± 364.46 | 6360 | 2060 | 723 | 2720 | 3360 | 2215.75 ± 1127.84 |
| 6 hr | 299 | 148 | 646 | 364.33 ± 255.34 | 11500 | 2090 | 2040 | 2490 | 3850 | 2617.5 ± 845.98 |
| 24 hr | 270 | 916 | 704 | 630 ± 329.29 | 7150 | 2420 | 1410 | 2340 | 3510 | 2420 ± 859.18 |
| Predose | 247 | 520 | 607 | 458 ± 187.83 | 1480 | 781 | 355 | 788 | 968 | 723 ± 260.15 |
| 6 hr | 240 | 462 | 485 | 395.66 ± 135.30 | 2230 | 698 | 446 | 749 | 878 | 692.75 ± 181.10 |
| 24 hr | 240 | 491 | 452 | 394.33 ± 135.07 | 2070 | 775 | 498 | 691 | 801 | 691.25 ± 137.11 |
| Predose | 403 | 823 | NS | 613 ± 296.98 | 5880 | NS | NS | 3110 | 4230 | 3670 ± 791.95 |
| 6 hr | 506 | 838 | NS | 672 ± 234.75 | 9370 | NS | NS | 3370 | 2790 | 3080 ± 410.12 |
| 24 hr | 432 | 665 | NS | 548.5 ± 164.75 | 6380 | NS | 1190 | 3020 | 4570 | 2926.66 ± 1691.93 |

NS: no sample collected;
QNS : Quantity not sufficient
Summary: Talabostat mesylate 0.3 mg BID (0.6 mg TDD) administered on days 1-14 and Pembrolizumab administered on 200 mg every 21 days. Splitting the TDD (totaling 0.6 mg) was associated with improved tolerability as evidenced by no reported DLTs and lower rates of other adverse events of interest such as hypotension and peripheral edema.

Phase 2—Efficacy Phase—Cohort B-Adenocarcinoma—

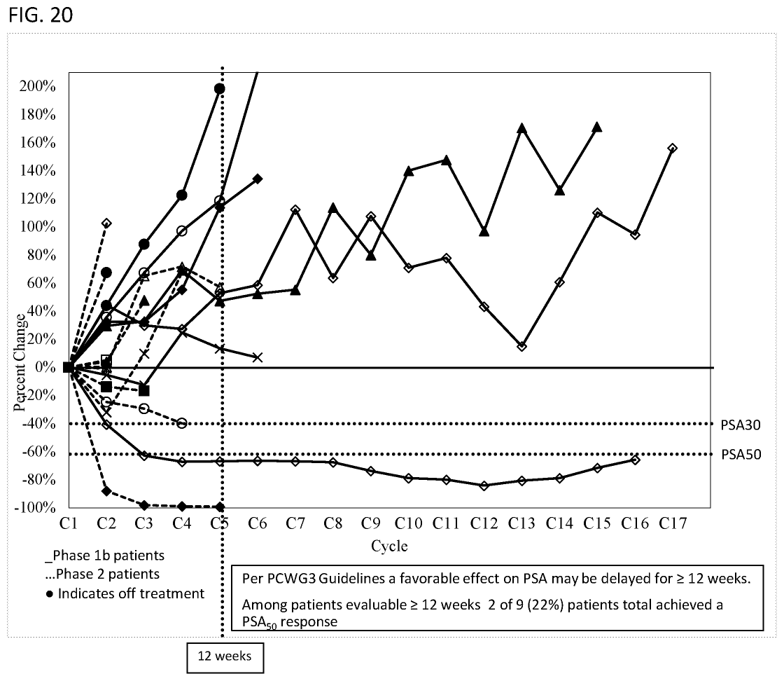

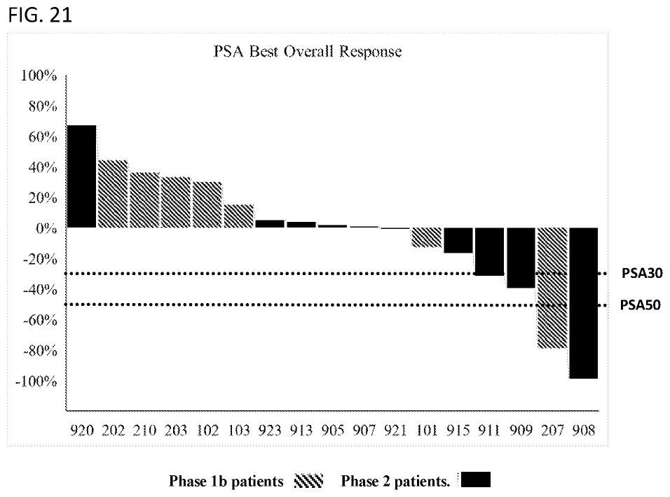

The Phase 2 efficacy portion of this study assessed the anti-tumor activity in patients with adenocarcinoma of the combination in a setting where checkpoint inhibitor monotherapies have demonstrated limited clinical benefit and the preliminary results are tabulated in Tables 11-14 and given in FIGS. 20 and 21. The study was expanded to include a cohort of mCRPC adenocarcinoma. Details of confirmed PSA responders:

Details of Responders of Phase 2—

Responder 1 details (PID 908)—PSA/CTC Response with Minor Response in Target Lesions of Phase 2

Patient Demographics and Treatment History:
68 y.o. Initial Dx 2017, mCRPC in 2019
Sites of disease: liver, lymph nodes and bone
Baseline PSA 25.0 ng/mL
Prior systemic therapy included leuprolide, sipuleucel, enzalutamide, docetaxel, cabazitaxel

On-Treatment Disease Status
Duration of Talabostat mesylate+Pembro: 5+ cycles—
   PSA response at 3 weeks, ongoing at 15 weeks (0.2 ng/mL −99% reduction)
CTC conversion at 3 weeks
RECIST 1.1 Status: Minor Response (on-going)
   19% reduction in Target Lesions (liver, lymph)

Responder 2 Details (PID 909)—Minor Response in Target Lesions of Phase 2—

Patient Demographics and Treatment History:
73 y.o. Initial Dx 2007, mCRPC in 2016
Sites of disease: lymph nodes
Baseline PSA 111.4 ng/mL
Prior systemic therapy included bicalutamide, leuprolide, enzalutamide, docetaxel, abiraterone+prednisone

On-Treatment Disease Status
Duration of Talabostat mesylate+Pembro: 4 cycles
   PSA decline ongoing at 12 weeks (66.9 ng/mL-40% reduction)
RECIST 1.1 Status: Minor Response (on-going)
   27% reduction in Target Lesions (lymph)

TABLE 11
Phase 2-Efficacy stage (Cohort B-adenocarcinoma)-Patient disease history

| | | Phase 2 |
|---|---|---|
| | | Adenocarcinoma |
| | | Cohort |
| Baseline Characteristics | | N (%) |
| Enrolled | | 15 |
| Age (years) | Mean (range) | 70 (58-80) |
| (n = 12) | | |
| ECOG | 0 | 7 (47%) |
| Performance | 1 | 7 (47%) |
| Status | Not available | 1 (6%) |
| Prior Systemic | 1st Generation androgen | 6 (46%) |
| (n = 13) | deprivation therapy | |
| Therapies | 2nd Generation androgen | 12 (92%) |
| | signaling inhibitor | |
| | Chemotherapy | 13 (100%) |
| | Other Systemic Therapies | |
| | PSMA based therapy | 1 (8%) |
| | Sipuleucel-T | 5 (38%) |

TABLE 12
Preliminary Activity Observed in Adenocarcinoma Population:

| | | |
|---|---|---|
| | Phase 1b Population | Phase 2 Population |
| | Total = 7 | Total = 15 |
| Best Response | n (%) | n (%) |

RECIST 1.1 by Investigator Assessment^{a}

| | | |
|---|---|---|
| Evaluable | 7 | 5 |
| Measurable | 4 (57%) | 4 (80%) |
| Best RECIST Response | | |
| PR | 1 (14%) | 0 |
| SD (any duration) | 3 (43%) | 3 (60%) |
| including Minor Response | | 2 (40%) |
| Non-CR/Non-PD | 3 (43%) | 1 (20%) |
| PD | 0 | 1(20%) |
| Disease Control Rate | 7 (100%) | 4 (80%) |
| PR + SD + Non-CR/Non-PD | | |

PSA

| | | |
|---|---|---|
| PSA Evaluable^{b} | 7 | 10 |
| PSA50 Response | 1 (14%) | 1 (10%) |

CTC

| | | |
|---|---|---|
| CTC Evaluable^{c} | 3 | 4 |
| CTC Response^{d} | 0 | 1 (25%) |
| Composite Response | 1 of 7 evaluable | 1 of 10 evaluable |
| | | to date |

^{a}Patients who received ≥ one dose of study therapy and 1 on-treatment tumor assessment;
^{b}Baseline value >4 ng/ml and one on-treatment PSA assessment
^{c}Baseline CTC value ≥ 5/7.5 mL and one measurable on-treatment assessment
^{d}CTC conversion from ≥5/7.5 mL to <5/7.5 mL

TABLE 13
Phase 2-Efficacy stage-Cohort B-Exposure Duration
and Subject Disposition

| | | | | | | |
|---|---|---|---|---|---|---|
| Pt # | C1D1 | C2D1 | C3D1 | C4D1 | C5D1 | C6D1 |

Phase 2 Adenocarcinoma cohort (N = 15)

| | | | | | | |
|---|---|---|---|---|---|---|
| 111-905 | X | X | DC | | | |
| 106-908 | X | X | X* | X* | X* | X* |
| 112-907 | X | X | X | X | X | |
| 112-909 | X | X | X | X* | DC | |
| 103-911 | X | X | X | X | | |
| 112-912 | X | DC | | | | |
| 112-913 | X | X | X | DC | | |
| 106-914 | X | | | | | |
| 106-915 | X | X | X | | | |
| 112-916 | X | DC | | | | |
| 106-920 | X | X | | | | |
| 108-921 | X | X | | | | |
| 108-922 | X | X | | | | |
| 101-923 | X | X | | | | |
| 101-924 | X | X | | | | |

*3 days on/2 days off dosing

Discontinuations for reasons other than clinical or disease progression:
Patient 112-909 Withdrew consent refused further treatment
Patient 112-912 Withdrew consent refused further treatment

### Patient 112-916 Investigator Decision

Median duration on-treatment: 8+ weeks (2.7+ cycles), range 1+ to 18+ weeks (1-6 cycles) 10 patients remain on treatment with 3 patients on treatment for >3 cycles

TABLE 14
Phase 2 Safety in Adenocarcinoma Population:

| | Grade | | | |
|---|---|---|---|---|
| Preferred Term | N = 13 | | | |
| Talabostat Related Events# | Grade 1 | Grade 2 | Grade 3 | Total |
| Subjects with any event | 12 | 7 | 4 | 13 |
| Hypotension* | 2 | 2 | 1 | 5 |
| Fatigue | 4 | 1 | — | 5 |
| Nausea | 4 | 1 | — | 5 |
| Dizziness | 1 | 2 | 1 | 4 |
| Vomiting | 4 | — | — | 4 |
| Rash | 3 | — | — | 3 |
| Decreased appetite | 3 | — | — | 3 |
| Decreased platelet count | 3 | — | — | 3 |
| Blood lactic acid increased | — | — | 2 | 2 |
| Myalgia | 1 | — | 1 | 2 |
| Chills | 2 | — | — | 2 |
| Fever | 2 | — | — | 2 |
| Constipation | 2 | — | — | 2 |
| Dry mouth | 2 | — | — | 2 |

#Relatedness as assessed by investigator,
*Includes orthostatic hypotension

Majority of events were low grade.
AEs consistent with cytokine activation were observed
Grade 3 hypotension was observed during the first week of treatment in a patient who initiated dosing with 0.3 mg BID.
   Step-up dosing was then implemented for all new patients with Talabostat mesylate 0.2 mg BID day 1 through day 7. Escalation to 0.3 mg BID was permitted if no treatment related AEs Grade >1 or skipped doses due to hypotension or orthostasis occurred during the first week of treatment.
   Other Talabostat Related Adverse Events were limited to Grade 1 hypoalbuminemia, AST increased, LDH increased, and hyponatremia in 2 patients each

Phase 2 summary: Orally administered Talabostat mesylate in combination with pembrolizumab demonstrated anti-tumor activity in some heavily pre-treated, refractory mCRPC with adenocarcinoma phenotype, a setting where checkpoint inhibitor monotherapies have demonstrated limited clinical benefit and patients have limited treatment options. Despite limited efficacy follow-up in the Phase 2 portion of the study at this data cut-off, 1 ongoing patient has achieved a PSA₅₀ response plus CTC response and has had a 19% decrease in target lesions. A second patient has had a 40% reduction in PSA with a 27% reduction in target lesions. The disease control rate, defined as PR+SD+non-CR/non-PD, is 80% among RECIST evaluable patients. In the Phase 1b, 1 ongoing patient with adenocarcinoma has a PSA₅₀ response and a RECIST 1.1 partial response. Efforts are underway to identify a potential predictive biomarker.

The BID dosing schedule for Talabostat mesylate continues to demonstrate an acceptable safety profile when given in combination with pembrolizumab with primarily low grade on-target adverse events consistent with cytokine activation. This study continues to enroll patients with adenocarcinoma and SC/t-NEPC as per protocol.

TABLE 15
Study Schedule of Assessments-

| | Screen | | | | | | | | | | | | | | | | | |
|---|---|---|---|---|---|---|---|---|---|---|---|---|---|---|---|---|---|---|
| | Period | | | | | | | | | | | | | | | | | |
| | D28 to | Cycle 1^{u} | | | | | | Cycle 2 | | | | | >Cycle 2 | | | | EOT | FU |
| Screen/Cycle Day (D): | D1 | D1^{c} | D2 | D4 (+1) | D8 | D14 | D15 | D1^{d} | D2 | D8 | D14 | D15 | D1^{d} | D8 | D14 | D15 | Visit^{a} | Visit^{b} |
| Informed consent | X | | | | | | | | | | | | | | | | | |
| Inclusion and | X | X | | | | | | | | | | | | | | | | |
| exclusion criteria | | | | | | | | | | | | | | | | | | |
| Demographics | X | | | | | | | | | | | | | | | | | |
| Adverse event assessment | X | X | X | X | X | | X | X | | X | | X | X | X^{e} | | X^{e} | X | X |
| Concomitant medications | X | X | | | X | | X | X | | X | | X | X | X^{e} | | X^{e} | X | X |
| Prostate cancer history | X | | | | | | | | | | | | | | | | | |
| and treatment^{f} | | | | | | | | | | | | | | | | | | |
| Archival tumor collection | X | | | | | | | | | | | | | | | | | |
| Central pathology | X | | | | | | | | | | | | | | | | | |
| review of primary | | | | | | | | | | | | | | | | | | |
| or metastatic prostate | | | | | | | | | | | | | | | | | | |
| cancer tissue^{g} | | | | | | | | | | | | | | | | | | |
| Imaging and Other | | | | | | | | | | | | | | | | | | |
| Bone scintigraphy | X | | | | | | | | | | | | X^{h} | | | | X^{h} | |
| CT/MRI | X | | | | | | | | | | | | X^{h} | | | | X^{h} | |
| Tumor assessment | X | | | | | | | | | | | | X^{h} | | | | X^{h} | |
| Study Drug Administration | | | | | | | | | | | | | | | | | | |
| Talabostat administration | | Day 1-14 | | | | | | Day 1-14 | | | | | Day 1-14 | | | | | |
| PEMBRO administrationⁱ | | X | | | | | | X | | | | | X | | | | | |
| Clinical Procedures | | | | | | | | | | | | | | | | | | |
| Physical examination | X | X | | | X | | X | X | | X | | X | X | | | | X | |
| Medical history/current | X | | | | | | | | | | | | | | | | | |
| medical conditions | | | | | | | | | | | | | | | | | | |
| ECOG performance status | X | X | | | X | | X | X | | X | | X | X | | | | X | |
| Vital signs are during the | X | X | X | X | X | X | X | X | | | X | X | X | | | | X | |
| dosing period (sitting and | | | | | | | | | | | | | | | | | | |
| standing after 5 | | | | | | | | | | | | | | | | | | |
| minutes of rest, BP, HR, | | | | | | | | | | | | | | | | | | |
| body temperature, RR) | | | | | | | | | | | | | | | | | | |
| Blood pressure | | | Daily, prior to each dose | | | | | | | | | | | | | | | |
| monitoring at home | | | | | | | | | | | | | | | | | | |
| Cycle 1 twice daily, prior | | | | | | | | | | | | | | | | | | |
| to each dose^{j} | | | | | | | | | | | | | | | | | | |
| Review patient BP | | | X | X | X | X | X | | | | | | | | | | | |
| log. Staff contacts | | | | | | | | | | | | | | | | | | |
| are through C1D14^{k} | | | | | | | | | | | | | | | | | | |
| Height | X | | | | | | | | | | | | | | | | | |
| Weight | X | X | | | | | | X | | | | | X | | | | X | |
| Metastatic tumor biopsy^{l} | X | | | | | | | | | | | | | | | | | |
| ECG^{m} | X | X | | | | | | X | | | | | X | | | | X | |
| Administration of IV fluidsⁿ | | X | | | | | | | | | | | | | | | | |
| Clinical Laboratory Tests | | | | | | | | | | | | | | | | | | |
| Hematology (CBC | X | X | | | X | | X | X | | X | | X | X | X^{o} | | X^{o} | X | |
| plus differential | | | | | | | | | | | | | | | | | | |
| [5-part or auto-analyzer]) | | | | | | | | | | | | | | | | | | |
| Serum chemistry^{p} | X | X | | | X | | X | X | | X | | X | X | X^{o} | | X^{o} | X | |
| Liver function tests^{q} | X | X | | | X | | X | X | | X | | X | X | X^{o} | | X^{o} | X | |
| TSH, free T3 and free T4^{r} | X | | | | | | | X | | | | | X | | | | X | |
| Serum PSA | X | X | | | | | | X | | | | | X | | | | | |
| Urinalysis | X | X | | | | | | X | | | | | X | | | | X | |
| Serum collection | | X | X | X | | X | X | X | X | | X | X | | | X | | | |
| for cytokines^{s} | | | | | | | | | | | | | | | | | | |
| Whole blood collection | | X | | | | X | | X | | | X | | | | | | | |
| for immune | | | | | | | | | | | | | | | | | | |
| parameters^{t} | | | | | | | | | | | | | | | | | | |
| Enumeration of CTCs by | | X | | | | | | X | | | | | X | | | | X | |
| Veridex assay^{u} | | | | | | | | | | | | | | | | | | |
| Pharmacokinetic | | X^{v} | | X^{v} | | X^{v} | | X^{v} | | | X^{v} | | X^{v} | | X^{w} | X^{w} | | |
| blood sampling | | | | | | | | | | | | | | | | | | |

Abbreviations: ALT = alanine aminotransferase; AST = aspartate aminotransferase; BP = blood pressure; BUN = blood urea nitrogen; Ca = calcium; Cl = chloride; Cr = creatinine; CxDx = Cycle (number) Day (number); CBC = complete blood count; CT = computed tomography; CTC = circulating tumor cell; ECG = electrocardiogram; ECOG = Eastern Cooperative Oncology Group; eCRF = electronic Case Report Form; EOT = End of Treatment; FU = Follow-Up; HR = heart rate; IV = intravenous; K = potassium; LDH = lactate dehydrogenase; Mg = magnesium; MRI = magnetic resonance imaging; MSI = microsatellite instability; Na = sodium; PD = progressive disease; PEMBRO = pembrolizumab; PSA = prostate-specific antigen; QTcB = QT interval corrected for heart rate using Bazett's formula; RR = respiratory rate.
^{a}After discontinuation of study drugs, patients completed an EOT visit within 21 days after the last study drug dose.
^{b}Safety Follow-up Visit was conducted 30 days (±7 days) after the last dose of study drug and later if drug-related AEs did not resolved at that time. Thereafter, patients without documented PD were followed every 90 days for disease assessments until documentation of PD. After documentation of PD, patients were followed every 90 days for survival status; such follow-up was conducted by telephone.
^{c}If Cycle 1 Day 1 occured <72 hours after Screening, physical examinations, clinical laboratory tests, and ECGs did not need to be repeated
^{d}Day 1 of Cycle 2 and all subsequent cycles will be 21 days (±3 days) after the previous Day 1 dose of study drug was administered.
^{e}For >Cycle 2, assessment could be performed via telephone if no other reason for clinic visit
^{f}If available, provided data on germline mutations or MSI/dMMr within eCRF
^{g}The results from the central pathology review were not required before enrollment if SCNC or adenocarcinoma without small cell neuroendocrine features confirmed on local pathology review. Central pathology review was optional for the safety lead-in
^{h}Tumor assessment must include cross-sectional imaging (MRI or CT scanning with IV contrast whenever possible) of the chest/abdomen/pelvis plus whole-body bone scan. Other body sites (e.g., neck) included as clinically indicated. Tumor assessment was performed at Screening, C4D1 (±7 days), C7D1 (±7 days), C10D1 (±7 days), and Day 1 (±7 days) of every 3rd cycle thereafter.
ⁱEither Talabostat mesylate or PEMBRO was administered first. However, on Cycle 1 Day 1, it was recommended that PEMBRO be administered first and that ≥1 hour should elapse before the administration of Talabostat mesylate so that it was easier to determine the relatedness of any AEs to study drug
^{j}Daily during the dosing period of Cycle 1, and during the first week of treatment at 0.3 mg BID (should this occur at a cycle later than Cycle 1), the patient performed at home blood pressure monitoring at least twice daily, once in the morning and once in the late afternoon/evening, prior to dosing. The patient recorded blood pressure measurements on a log and was asked to bring the log with them to every clinic visit during Cycle 1 and during the first cycle that their dose was escalated to 0.3 mg BID (should this occur at a cycle later than Cycle 1). The patient was instructed that they must not take their dose if their blood pressure was below 100 mmHg systolic or 50 mmHg diastolic. The patient must report any values below this to study physician immediate.
^{k}During the first week of treatment in Cycle 1, through Day 14, the study team maintained regular daily contact with the patient to remind them of oral hydration guidelines, review the side effects, and review at-home blood pressure measurements.
^{l}Tumor biopsy was optional in the Lead-In Stage and mandatory in the Efficacy Stage. Requirement was waived if there was no safely accessible lesion OR patient had available archival metastatic tumor tissue.
^{m}All ECGs were performed in triplicate, per local standard practice, prior to collection of blood samples. At Screening only, QTcB was measured.
ⁿAdministration of at least 1 L of IV fluids was required at Cycle 1 Day 1. It was at the investigator's discretion to provide IV hydration during in-person clinic visits beyond Cycle 1 Day 1
^{o}Cycle 3 and subsequent cycles, Day 8, and Day 15: For patients who did not experienced a laboratory abnormality since the start of treatment, or who had no worsening of baseline laboratory abnormality grade, the clinical laboratory assessments (hematology, serum chemistry, and liver function tests) on Day 8 and Day 15 of each cycle were not required. These patients continued clinical laboratory assessments on Day 1 of each cycle as required. For patients who experienced Grade >3 laboratory abnormalities after the start of treatment at Day 1 of any cycle beyond Cycle 3, these patients continued Day 8 and Day 15 clinical laboratory assessments until the resolution of the laboratory abnormality
^{p}Serum chemistry included: Na, K, Cl, bicarbonate, Ca, Mg, phosphate, BUN/Cr, and LDH. At Screening and D1 of every cycle visit, a blood lactic acid and glucose test was performed.
^{q}Liver function tests included aspartate AST, ALT, alkaline phosphatase, total bilirubin, and albumin.
^{r}TSH, free T3 and free T4 at screening only. TSH, with reflexive free T3 and free T4 if TSH is abnormal, to be performed every other cycle (2, 4, 6, etc.) and EOT visit
^{s}Serum collection for cytokines occured predose (recommended within 30 minutes prior to dosing) and 6 (±1 hour) and 24 (±4 hours) hours post dose on C1D1, C1D14, C2D1, and C2D14 and at C3D14 at Predose (recommended within 30 minutes prior to dosing) and 6 hours (±1 hour) after the morning dose. The Day 1, 24-hour samples were collected prior to the Day 2 morning dose. The Day 1, 24 hour samples was collected prior to the Day 2 morning dose. On Cycle 1 Day 4 (+1) only, a serum collection was performed for cytokines predose (recommended within 30 minutes prior to dosing).
^{t}Whole blood was collected predose on Cycle 1 Day 1, Cycle 1 Day 14, Cycle 2 Day 1, and Cycle 2 Day 14 and assessed for leukocytes by flow cytometry (recommended within 30 minutes prior to dosing)
^{u}Enumeration of CTCs was performed at predose (−2 hours) on CID1, C2D1, and C4D1, and then on DI of every third cycle thereafter, and at the EOT visit (anytime).
^{v}PK Sample was collected predose (within 24 hours prior to dose) C1D1, C2D1 and C3D1. Sample was collected within 30 minutes before morning dose was administered on C1D14 and C2D14(patient diary was kept to record the number of doses the patient had taken in the cycle). On Cycle 1 Day 4 (+1) only, collected a PK sample predose (recommended within 30 minutes prior to dosing)
^{w}Cycle 3 only; samples were collected within 30 minutes before the morning dose was administered on Day 14 and at 2 hours (±15 minutes), 6 hours (±30 minutes), and if possible, 10-12 hours* (*optional draw if patient or site could not accommodate). Additional samples were collected at these approximate times after the Day 14 evening dose: 10-14 hours, 60 hours, 108 hours, and 156 hours. The 156-hour sample was collected within 30 minutes before the C4D1 dose. If C4 was not administered, the sample was collected 156 hours after the last dose (±5 hours) (patient diary was kept to record the number of doses the patient had taken in the cycle).

### Example 2: Phase 2 Basket Study of Talabostat Mesylate, a Small Molecule Inhibitor of Dipeptidyl Peptidases (DPP), Administered in Combination with Pembrolizumab in Patients with Advanced Solid Cancers

STUDY OBJECTIVE(S): The primary objectives of the study were:
To evaluate response rate per Response Evaluation Criteria in Solid Tumors (RECIST) and immune iRECIST in patients treated in cohort A and in patients treated in cohort B. Tumor measurements were in QIAC.
To evaluate dose-limiting toxicities (DLT) in the first 6 patients enrolled to the study.

The secondary objectives of the study for cohort A and cohort B included:
To evaluate progression-free survival (PFS)
To evaluate duration of response (DOR)
To evaluate overall survival (OS)
To evaluate overall safety and tolerability

Exploratory Objectives:
To evaluate the quantitative and qualitative effects of Talabostat mesylate in combination with pembrolizumab on relevant immune effector cells and cytokines in tumor and blood respectively.
To evaluate the quantitative and qualitative effects of Talabostat mesylate in combination with pembrolizumab on various immunological effector cells, including neutrophils, myeloid derived suppressor cells (MDSCs), dendritic cells, cancer associated fibroblast (CAF), T-cells and macrophage density in pre-dose tumor biopsies and when feasible in post-dose tumor tissues.
To explore the predictive value of baseline PD-L1 tumor expression and tumor mutation burden (TMB) with clinical outcomes
To evaluate changes in serially collected blood circulating tumor DNA (ctDNA) to assess for tumor response and clonal evolution
To evaluate pre- and post-treatment PD-L1 PET/CT as a predictive tool for therapeutic efficacy.

STUDY DESIGN: This was an open-label, single-institution, Phase 2 study to determine the response rate of Talabostat mesylate administered orally and daily, combined with pembrolizumab, in patients with advanced solid cancers. The study also assessed other efficacy parameters, such as PFS, OS and DOR, as well as the safety of the combined treatment. Bayesian optimal phase 2 (BOP2) design was adopted to monitor efficacy. The study consisted of 2 stages:

1) Lead-in Stage (first 6 patients enrolled)—in which the safety and tolerability of the combination of Talabostat mesylate administered orally twice daily on Days 1 to 14 of a 21-day cycle plus pembrolizumab 200 mg administered intravenously (IV) over 30 minutes on Day 1 every 21 days was assessed and confirmed in patients with advanced solid cancers. The dose of Talabostat mesylate was 0.6 mg divided in two doses (0.3 mg twice daily (BID)).

2) Efficacy Stage (BOP2-Stage)—in which patients with advanced solid cancers were treated with Talabostat mesylate combined with pembrolizumab. Patients enrolled to the Lead-in Stage were also evaluated in the efficacy stage. New patients enrolled were administered Talabostat mesylate at the starting dose of 0.2 mg orally twice a day for the first 7 days. If well tolerated and in the absence of signs and symptoms of clinically significant hypotension the dose was escalated to 0.3 mg orally twice a day.

During the Lead-in Stage, patients were observed for dose-limiting toxicity (DLT) during Cycle 1. Six patients were treated initially with 0.3 mg BID Talabostat mesylate daily (Days 1 to 14) plus pembrolizumab 200 mg:
If >1 of the 6 original patients has a DLT in Cycle 1, the dose was considered above the maximum tolerated dose (MTD) and additional 6 patients were treated at the 0.4 mg Talabostat mesylate daily Days 1 to 14 dose level.
If ≤1 of the patients experienced a DLT, the Efficacy Stage could commence
If >1 of the patients experienced a DLT, a discussion was held between the investigators and supporters as to how to proceed

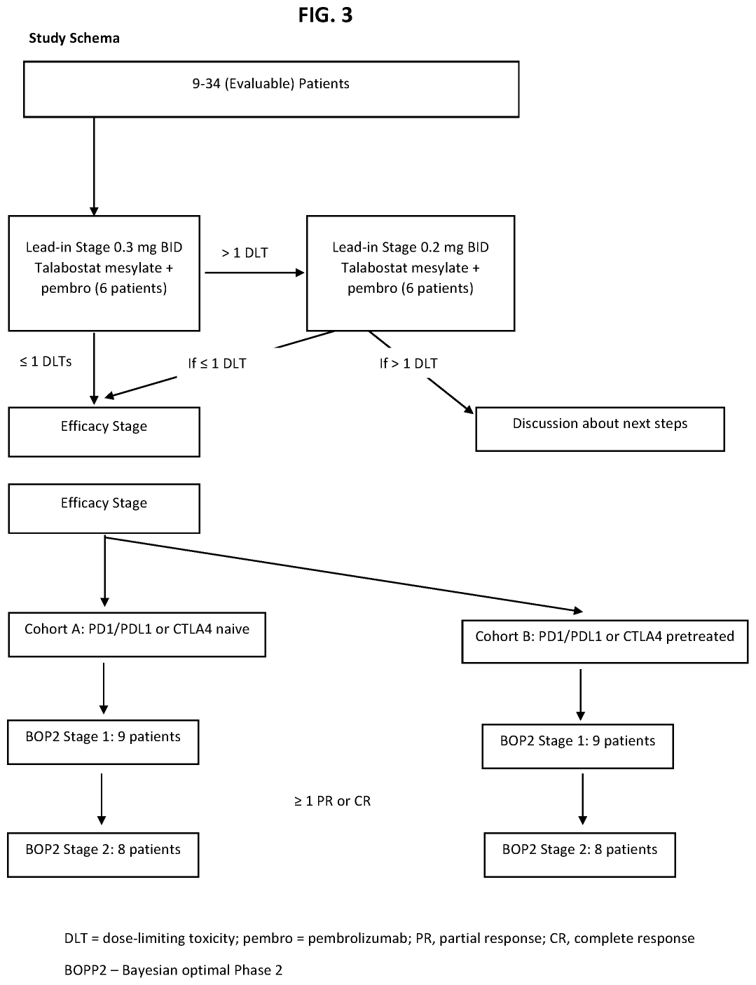

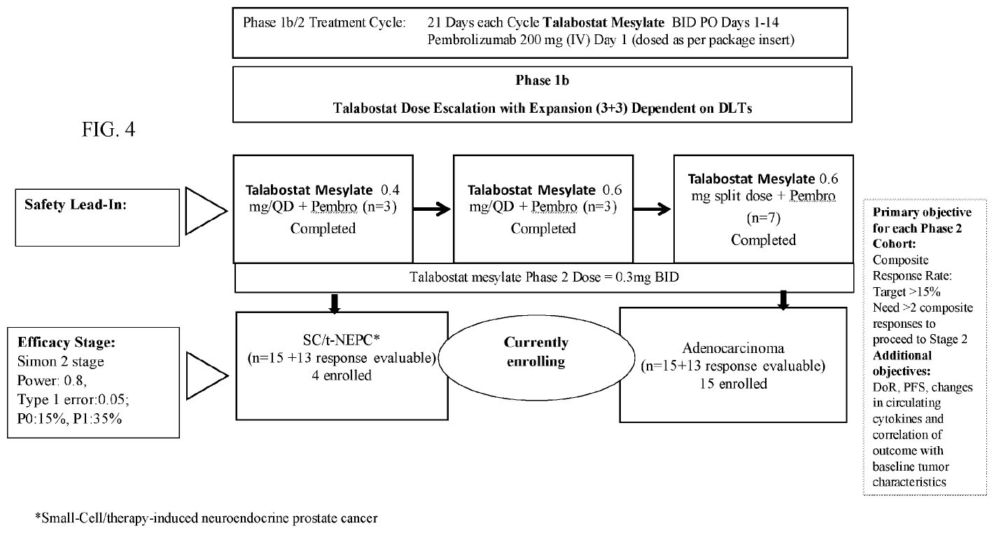

The study schema is presented in FIG. 3.

All safety data from all patients, who received at least one dose of study drug were included in safety analysis. Unless doses were held because of DLT, a patient must have received >70% of their Talabostat mesylate in Cycle 1 (i.e., ≥30 of 42 planned doses) with pembrolizumab dosed on Day 1 of Cycle 1 to be eligible for DLT assessment.

Toxicities were assessed by the investigator using the National Cancer Institute Common Terminology Criteria for Adverse Events (NCI CTCAE), version 5. The relationship of an AE to combination therapy (i.e., attribution to Talabostat mesylate and/or pembrolizumab) was assessed by the investigator using the criteria in the protocol.

A DLT was defined as any of the following AEs occurring during Cycle 1, regardless of investigator attribution to study treatment, unless the AE could be clearly and incontrovertibly attributed to an extraneous cause (e.g., disease progression) by the Principal Investigator:
Any Grade 4 laboratory abnormality, regardless of duration
Any Grade 3 laboratory abnormalities if associated with clinical symptoms regardless of duration
Any Grade 3 non-hematologic AE, with the exceptions of Grade 3 nausea, vomiting, diarrhea, constipation, fever, fatigue that resolves to Grade≤1 within 72 hours with optimal medical management and/or supportive measures.
Grade≥3 thrombocytopenia with Grade>1 bleeding or requirement for platelet transfusion.
Grade≥3 febrile neutropenia.
Grade≥3 fever.
Grade>3 skin rash.
Laboratory abnormalities meeting Hy's law criteria (aspartate aminotransferase [AST] or alanine aminotransferase [ALT]>3×upper limit of normal [ULN] with concomitant total bilirubin >2×ULN).
Any toxicity resulting in ≥30% held/skipped doses of Talabostat mesylate during Cycle 1.
Delay of Cycle 2 by ≥14 days due to toxicity.
Any other significant toxicity considered by the investigator and supporter's medical representatives to be dose-limiting.

Efficacy Stage:

After assessment of the safety and confirmation of the Talabostat mesylate/pembrolizumab dose schedule to be used in the subsequent stage, the Efficacy Stage begun. Eligible patients received oral Talabostat mesylate daily on Days 1 to 14 of a 21-day cycle plus pembrolizumab 200 mg administered IV on Day 1 every 21 days.

Study Design Features (Both Stages):

In both Lead-in and Efficacy Stages, patients were screened for study eligibility within 28 days before the first study drug dose after provision of written informed consent. Patients who were determined to be eligible, based on Screening assessments, were enrolled in the study on Cycle (C)1, Day (D)1 (Baseline, before the first dose of Talabostat mesylate).

During treatment, patients attended study center visits and had study evaluations performed as detailed in the Schedule of Assessments (Table 18). All study visits were conducted on an outpatient basis but might be conducted on an inpatient basis per the investigator's judgement.

All patients had pre-treatment (prior to study treatment dosing) imaging (computed tomography [CT] scan of chest/abdomen/pelvis or magnetic resonance imaging [MRI] for baseline tumor measurements, as well as bone scintigraphy [BS]). Patients with skin, subcutaneous or lymph node metastases might also had tumor evaluations (including measurements, with a ruler) by means of physical examination. Patients with a history of central nervous system (CNS) malignant involvement or CNS symptoms had either CT or MRI imaging of the brain performed to assess active CNS malignancy.

Tumor measurements and disease response assessments (CT or MU; BS) were also to be performed at the end of Cycle 3 (approximately 9 weeks after the first study treatment dose), and then approximately every 9 weeks thereafter until development of progressive disease (PD). Intervals could be shortened to 6 weeks if clinically necessary per treating physician. For patients with evidence of disease control (stable disease or better) at Week 27, tumor measurements and disease response assessments might be performed less frequently (approximately every 12 weeks) thereafter. Tumor measurements and disease response assessments also were to be performed at the End of Treatment (EOT) visit.

Study Population

Approximately 6 to 12 and 24 to 48 patients who fulfilled the eligibility criteria of the protocol were enrolled during Lead-in and Efficacy Stages of the protocol, respectively. Patients enrolled to the Lead-in Stage were evaluated and used for the efficacy stage.

Eligibility Criteria

All patients must satisfy the following inclusion and exclusion criteria to be eligible for entry into the trial.

### Inclusion Criteria

1. Patient with a histology or cytology proven solid advanced cancer, which failed or was intolerant of standard therapies known to offer survival benefit unless standard therapies include PD1 or PD-L1 antibodies.
   a. Lead-in stage: patient with advanced cancers meeting the criteria above with or without prior treatment with PD1/PDL 1 and/or CTLA-4 targeted therapies. Patients with prior treatment with PD1/PDL1 targeted therapies should be relapsed.
   b. Efficacy stage cohort A: patients with advanced cancers not previously treated with PD1/PDL1 or CTLA4 targeted therapies.
   c. Efficacy stage cohort B: patients with advanced cancers which had relapsed or progressed with PD1/PDL1 or CTLA4 targeted therapies.
2. Patient with a life expectancy of more than 3 months, in the opinion of the investigator.
3. Patient had Eastern Cooperative Oncology Group (ECOG) performance status of 0-2.
4. Patient was >12 years of age. Patients <18 years of age had to weigh ≥40 kgs
5. Patients must had measurable disease by RECIST 1.1 and iRECIST. Disease amenable to a biopsy was not mandatory. However, pre- and post-treatment biopsies were mandatory if deemed medically safe and feasible for arms, which advanced to Stage 2 of Efficacy Stage (BOP2).
6. Patient's acute toxic effects of previous anticancer therapy had resolved to ≤Grade 1 except for Grade 2 peripheral neuropathy or any grade of alopecia.
7. Patient had an adequate baseline organ function, as demonstrated by the following:
a. Serum creatinine <1.5 times institutional upper limit of normal (ULN) or calculated creatinine clearance >40 mL/min;
b. Serum albumin ≥2.5 g/dL;
c. Total bilirubin ≤1.5×ULN (for patients with known Gilbert syndrome ≤3×ULN);
d. Aspartate aminotransferase (AST) and alanine aminotransferase (ALT)≤3×institutional ULN (patients with hepatic metastasis must had AST/ALT≤5×ULN).
8. Patient had adequate baseline hematologic function, as demonstrated by the following:
a. Absolute neutrophil count (ANC)≥1.0×10⁹/L.
b. Hemoglobin ≥8 g/dL and no red blood cell transfusions during the prior 7 days.
c. Platelet count ≥75×10⁹/L.
9. Women of childbearing potential must had a negative serum or urine pregnancy test (minimum sensitivity 25 IU/L or equivalent units of human chorionic gonadotropin [HCG]) within 14 days prior to the initiation of treatment and/or postmenopausal women must be amenorrheic for at least 12 months to be considered of non-childbearing potential. Women of childbearing potential must agree and committed to the use of 2 highly effective methods of birth control throughout the duration of the study until at least 4 months following the last dose of study drug. Acceptable methods were defined as those that result, alone or in combination, in a low failure rate (i.e., less than 1% per year) when used consistently and correctly, such as surgical sterilization, an intrauterine device, or hormonal contraception in combination with a barrier method. It was currently unknown whether Talabostat mesylate or pembrolizumab might reduce the effectiveness of systemically acting hormonal contraceptives; therefore, women using systemically acting hormonal contraceptives should add a barrier method. In certain countries (if permitted by law), WOCBP may agree to abide by heterosexual sexual abstinence during the time of participation in this study.
10. Male patients and their female partners of childbearing potential must agree and commit to use a barrier contraception (e.g. condom with spermicidal foam/gel/film/cream/suppository) throughout the duration of the study until at least 60 days following the last dose of study drug, in addition to their female partners using either an intrauterine device or hormonal contraception and continuing until at least 4 months following the last dose of study drug. This criterion might be waived for male patients who had a vasectomy >6 months before signing the ICF.
11. Patients signed informed consent prior to initiation of any study-specific procedures or treatment.
12. Patient was able to adhere to the study visit schedule and other protocol requirements.

Exclusion Criteria
1. Patient could not swallow oral medication.
2. Patient was on gliptins.
3. Patient had active central nervous system (CNS) metastases not controlled by prior surgery or radiotherapy (patient must be off steroids). Patients with signs or symptoms suggestive of brain metastasis are not eligible unless brain metastases were ruled out by brain MRI/CT.
4. Patient had received external-beam radiation or another systemic anticancer therapy within 14 days or 5 half-lives, whichever was shorter, prior to study treatment.
5. Patient had received treatment with an investigational systemic anticancer agent within 14 days prior to study drug administration.
6. Patient had an additional active malignancy that might confound the assessment of the study endpoints. Patients with the following concomitant neoplastic diagnoses were eligible: non-melanoma skin cancer and carcinoma in situ (including transitional cell carcinoma, anal carcinoma, and melanoma in situ). Patients with simultaneous cancers, which were not active and did not require treatment might be eligible contingent on discussion with the PI and supporter.
7. Patient had clinically significant cardiovascular disease (e.g., uncontrolled or any New York Heart Association Class 3 or 4 congestive heart failure, uncontrolled angina, history of myocardial infarction, unstable angina or stroke within 6 months prior to study entry, uncontrolled hypertension or clinically significant arrhythmias not controlled by medication).
8. Patients with a history of clinically significant hypotension within past 3 months.
9. Patients, who were unable to maintain adequate hydration of ≥2 liters per day.
10. Patient had a diagnosis of immunodeficiency or was receiving systemic steroid therapy at a prednisone equivalent dose of >10 mg daily for at least 1 week or other form of immunosuppressive therapy within 7 days prior to Cycle 1 Day 1 (C1D1).
11. Patient had uncontrolled intercurrent illness including, but not limited to, uncontrolled infection, disseminated intravascular coagulation, or psychiatric illness/social situations that limit compliance with study requirements.
12. Patient had known positive status for human immunodeficiency virus active or chronic Hepatitis B or Hepatitis C. Patients with history of hepatitis B or C and undetectable viral load are eligible. Screening was not required.
13. Had a clinically significant upper gastrointestinal obstruction, abnormal physiological function or malabsorption syndrome that might affect the absorption of the study medication.
14. Patient had any medical condition which, in the opinion of the investigator, placed the patient at an unacceptably high risk for toxicity.
15. Patient was pregnant or breast-feeding

### Study Methodology

Permitted Medications/Therapies

The use of growth factors (e.g., granulocyte-colony stimulating factor [G-CSF]) was allowed as clinically indicated for the treatment of Grade ≥3 cytopenias.

Suggested supportive care measures for the management of AEs with potential immunologic etiology were outlined below. Where appropriate, these guidelines included the use of oral or IV treatment with corticosteroids as well as additional anti-inflammatory agents if symptoms did not improve with administration of corticosteroids. Note that several courses of steroid tapering might be necessary as symptoms might worsen when the steroid dose was decreased. For each AE, attempts was made to rule out other causes such as metastatic disease or bacterial or viral infection, which might require additional supportive care.

Prohibited Medications/Therapies

Enrolled patients might not receive investigational or approved anticancer agents including cytotoxic chemotherapy agents, anticancer tyrosine kinase inhibitors, or therapeutic monoclonal antibodies.

Concurrent radiation was not permitted with the exception of palliative radiation of limited number of isolated solitary lesions

Preclinical studies had demonstrated a low potential for Talabostat mesylate to inhibit the following major human liver CYP isoenzymes: CYP1A2, CYP2C8, CYP2C9, CYP2C19, CYP2D6, and CYP3A4. Further, relevant concentrations of Talabostat mesylate did not induce CYP3A4 or CYP1A2. Therefore, there were no prohibited medications based on CYP isoenzymes.

Efficacy Assessments

Efficacy was assessed during treatment using the RECIST 1.1 and iRECIST every 9 weeks (every 3 cycles). Intervals could be shortened to 6 weeks (every 2 cycles) if clinically necessary per treating physician. Tumor measurements were in QIAC.

Safety Assessments

Non-Serious Adverse Events

Investigators should assess for AEs at each visit. All AEs, including observed or volunteered problems, complaints, or symptoms, were to be recorded on the eCRF. Each AE was to be evaluated for duration, intensity, and causal relationship with the study treatment or other factors. MOCLIA was used for eCRF in this study.

The investigator was responsible for monitoring the safety of patients who entered the study. All AEs occurring during the treatment period and/or occurring within 30 days of the last dose of Talabostat mesylate and or pembrolizumab (investigational products, IPs) were followed to the end of the study or until resolution. AEs were graded according to the revised NCI CTCAE, Version 5.0, (see http://ctep.cancer.gov/reporting/ctc.html). AEs occurring 30 days after the last dose of IPs did not need to be reported unless the investigator considered the event to be related to IPs.

TABLE 16
Recommended Adverse Event Recording Guidelines

| Attribution | Grade 1 | Grade 2 | Grade 3 | Grade 4 | Grade 5 |
|---|---|---|---|---|---|
| Unrelated | Phase I | Phase I | Phase I | Phase I | Phase I |
| | | | Phase II | Phase II | Phase II |
| | | | | Phase III | Phase III |
| Unlikely | Phase I | Phase I | Phase I | Phase I | Phase I |
| | | | Phase II | Phase II | Phase II |
| | | | | Phase III | Phase III |
| Possible | Phase I | Phase I | Phase I | Phase I | Phase I |
| | Phase II | Phase II | Phase II | Phase II | Phase II |
| | | Phase III | Phase III | Phase III | Phase III |
| Probable | Phase I | Phase I | Phase I | Phase I | Phase I |
| | Phase II | Phase II | Phase II | Phase II | Phase II |
| | | Phase III | Phase III | Phase III | Phase III |
| Definitive | Phase I | Phase I | Phase I | Phase I | Phase I |
| | Phase II | Phase II | Phase II | Phase II | Phase II |
| | | Phase III | Phase III | Phase III | Phase III |

Reporting Serious Adverse Events

An adverse event or suspected adverse reaction was considered “serious” if, in the view of the investigator, it resulted in any of the following outcomes:
Death
A life-threatening adverse drug experience—any adverse experience that placed the patient, in the view of the initial reporter, at immediate risk of death from the adverse experience as it occurred. It did not include an adverse experience that, had it occurred in a more severe form, might have caused death.
Inpatient hospitalization or prolongation of existing hospitalization
A persistent or significant incapacity or substantial disruption of the ability to conduct normal life functions.
A congenital anomaly/birth defect.

Important medical events that might not result in death, be life-threatening, or required hospitalization might be considered a serious adverse drug experience when, based upon appropriate medical judgment, they might jeopardize the patient or subject and might require medical or surgical intervention to prevent one of the outcomes listed in this definition.

Serious adverse events were captured from the time of the first protocol-specific intervention, until 30 days after the last dose of drug or earlier if the participant withdrew consent or started a new anti-cancer therapy. Serious adverse events must be followed until clinical recovery was complete and laboratory tests returned to baseline, progression of the event had stabilized, or there had been acceptable resolution of the event.

Additionally, any serious adverse events that occurred after the 30-day time period that were related to the study treatment were reported to the IND Office. This included the development of a secondary malignancy.

### Adverse Event Follow-Up

Patients were to be monitored for AEs throughout the treatment period and for a minimum of 30 days after their last dose of Talabostat mesylate.

No further reporting of new AEs was required after the initiation of any subsequent chemotherapy or more than 30 days following the last dose of study medication, unless the study medication was considered to have contributed to the new AE.

### Pharmacokinetic Assessments/Pharmacodynamic Assessments

Whole blood samples (optional for Lead-in Stage and Stage 1 of Efficacy Stage [BOP1]; mandatory for Stage 2 of Efficacy Stage [BOP2]) were collected at the time points described in Table 18 (Examples of analysis included relevant immune effector cytokines, target engagement, testing of circulating tumor DNA (ctDNA).

### Disease Progression

Removal of Patients from the Study

Every effort within the bounds of safety and patient choice was made to have each patient complete the treatment period of the study. Patients who had treatment discontinued due to PD might be treated with any additional therapy deemed appropriate by the investigator. Patients with disease progression per RECIST and/or iRECIST can continue on therapy for clinical benefit if deemed appropriate by the investigator.

Patients might discontinue from the study for any of the following reasons:
Investigator recommended discontinuation and documented the reason(s)
There was a need for any treatment not allowed by the protocol
Patient's decision to withdrew consent or discontinue for any reason
There was an unacceptable AE thought to be related to study medication

Any patient who discontinued during the treatment period should return to complete safety and disease assessments (Table 18).

### Study Completion

The study was considered complete when all patients had followed to disease progression; were lost to follow-up, death, or withdrawal due to toxicity; patient's request; or investigator's discretion; and had completed all end-of-study treatment procedures.

### Study Medication

Study medication was administered in 21-day cycles. Either Talabostat mesylate or Pembrolizumab may be administered first. However, on Cycle 1 Day 1, it was recommended that Pembrolizumab be administered first and that ≥1 hour should elapse before the administration of Talabostat mesylate so that it was easier to determine the relatedness of any AEs to study drug.

Talabostat Mesylate Dosage and Administration

Talabostat mesylate tablets contained valine-proline boronic acid formulated as the methanesulfonate salt.

Current dosage strengths included 0.05 mg, and 0.2 mg tablets of Talabostat mesylate for oral administration.

Talabostat mesylate was administered orally as 0.2 mg, and 0.05-mg tablet. For a dose of 0.3 mg, patients took 1×0.2 mg tablet and 2×0.05 mg tablets together. Patients took 0.3 mg of the study drug twice a day, on Days 1 to 14 of each cycle, for a total daily dose of 0.6 mg.

Talabostat mesylate was continued until disease progression or unacceptable toxicity. Newly enrolled patients were administered Talabostat mesylate at the starting dose of 0.2 mg orally twice a day. If well tolerated and in the absence of signs and symptoms of clinically significant hypotension the dose was escalated to 0.3 mg orally twice a day.

On days when pharmacodynamic studies were being performed, Talabostat mesylate was administered at the study center, and administered at (approximately) the same time of day on each treatment day in the cycle. In cycles in which pharmacodynamics were not evaluated, Talabostat mesylate also was administered at (approximately) the same time of day on each treatment day in the cycle, preferably 0800 hours. If the patient forgot to take study medication, the dose was skipped.

Dose Adjustments of Talabostat Mesylate Secondary to Toxicity

Talabostat mesylate dose modifications within a treatment cycle were discouraged in Cycle 1 unless required by AE and/or DLT. In Cycle ≥2, dose modifications within a treatment cycle were at the discretion of the investigator. Doses held because of AEs should not be made up on subsequent days within or following a cycle. A dose that was missed for reasons other than an AE (i.e., the patient forgets to take a dose) might be administered on days subsequent to scheduled doses; any such adjustments were discussed with the Investigator. Under no circumstances should missed doses be made-up on a day when the patient was already taking a planned dose (i.e., no “doubling-up” to account for missed doses).

Recommendations for Talabostat mesylate dose modifications were:
Do not administer Talabostat mesylate if systolic blood pressure obtained before dosing was less than 100 mmHg
Grade 2 or higher AEs of edema/peripheral swelling, hypotension, dizziness, and hypovolemia:
For Grade 3 AEs, Held Talabostat mesylate until resolution of these AEs to ≤Grade 1 or baseline. Thereafter, restarted Talabostat at a dose reduced by 0.2 mg (reduction of 0.1 mg twice day to 0.2 mg twice a day; 1 dose level reduction).
For Grade 2 AEs, Held Talabostat mesylate until resolution of these AEs to <Grade 1 or baseline. Thereafter, restarted Restart Talabostat mesylate at the full dose or at a dose reduced by 0.2 mg (reduction of 0.1 mg twice day to 0.2 mg twice a day; 1 dose level reduction) at the discretion of the Investigator. For any Grade 3 or higher edema or Grade 2 edema that did not improved within 7 days, Talabostat mesylate was restarted at a dose reduced by 0.2 mg (1 dose level reduction) after resolution of the edema to <Grade 1 or baseline. If Grade 2 or greater edema did not recur during the next dosing period (2 weeks on/1 week off), the dose of Talabostat mesylate could be re-escalated at the Investigator's discretion.
For other Grade 2 or higher AEs deemed related to Talabostat mesylate:
Held Talabostat mesylate until resolution of these AEs to <Grade 1 or baseline.
For Grade 2 AEs, Restarted Talabostat mesylate at the full dose or at a dose reduced by 0.2 mg (1 dose level reduction) at the discretion of the Investigator.
For Grade 3 AEs, restarted Talabostat mesylate at a dose reduced by 0.2 mg (reduction of 0.1 mg twice day to 0.2 mg twice a day; 1 dose level reduction).
Once Talabostat mesylate dose has been de-escalated, it may not be re-escalated.
A maximum of 2 dose reductions per participant was permitted for Talabostat mesylate-related AEs, after which the study drug will be permanently discontinued.
Adverse events deemed related to Talabostat mesylate not recovering to ≤Grade 1 or baseline within 6 weeks from onset required permanent discontinuation of Talabostat mesylate.
Discontinued Talabostat mesylate for any life-threatening AE.

If an SAE thought to be related to Talabostat mesylate occurred during the Treatment Period, dosing of Talabostat mesylate was interrupted in that patient until the SAE resolved. If the Investigator wished to continue the patient on Talabostat mesylate, the supporter was contacted to discuss continuing Talabostat mesylate at the same or reduced dose.

If Talabostat mesylate was discontinued due to an AE, all termination from treatment procedures and assessments must be performed.

Monitoring of Patient Compliance with Talabostat Mesylate Study Medication

All Talabostat mesylate dosing containers must be returned to the clinic at each visit. Patients should be queried regarding their compliance with the dosing regimen and medication containers should be reviewed at each visit to determine if any doses of Talabostat mesylate had been missed, and the number of missed doses recorded. Patients must be at least 70% compliant with taking Talabostat mesylate in Cycles 1 and 2 in order to be included in the per-protocol efficacy analyses.

Talabostat Mesylate Description and Storage

Talabostat mesylate was supplied as 0.2-mg and 0.05-mg tablets in high-density polyethylene bottles with desiccant and child-resistant caps. The 0.2-mg strength was supplied as 30 tablets per bottle. The 0.05-mg strength was supplied as 90 tablets per bottle.

Supplies of Talabostat mesylate were appropriately labeled for clinical trial material. Talabostat mesylate was stored under refrigerated conditions between 2° C. to 8° C. (36° F. to 46° F.).

Pembrolizumab Administration, Dose Modifications and Discontinuation

Pembrolizumab was prepared, stored, and administered according to the current full Prescribing Information. Pembrolizumab was obtained from commercial supplies and was administered 200 mg intravenously over 30 minutes through a 0.2 to 5 micron sterile, nonpyrogenic, low-protein binding online or add-on filter. No other medication was infused through the infusion line. Infusion was interrupted and slowed for grade 1 or 2 infusion-related reactions and permanently discontinued for grade 3 or 4 infusion-related reactions. Pembrolizumab was administered until disease progression, unacceptable toxicity, or withdrawal of consent.

AEs associated with pembrolizumab exposure might be immune-mediated. Immune-related AEs might occur any time after pembrolizumab administration and may affect multiple body systems. Early recognition and treatment were important to reduce complications. Most immune-related AEs were reversible and could be managed with discontinuation of pembrolizumab and initiation of steroids. Patients who required a dose hold of pembrolizumab of ≥42 days were discontinued from the study.

Pembrolizumab should not be used in conjunction with other immunosuppressive agents other than corticosteroids administered for control of immune reactions considered related to pembrolizumab.

Data Analysis and Statistical Considerations

This was a phase 2, single center, basket study of oral Talabostat mesylate daily on days 1-14 in combination with intravenous PD1/PDL1 antibody on day 1 of 21-day cycle in subjects with advanced and refractory malignancies. Lead-in cohort enrolled 6 patients. Only the 6 patients treated at the selected dose during safety lead-in were assigned to Cohort A or B as appropriate. That is, if there was a dose de-escalation during safety lead-in, then the 6 patients treated at the higher dose was not be assigned to the phase II cohorts. Cohorts A and B enrolled 9 to 17 patients. Response assessments with CT and/or MRI were done every 9 weeks (3 cycles) following RECIST and iRECIST criteria. Intervals could be shortened to 6 weeks (2 cycles) if clinically necessary per treating physician.

The study followed the BOP2 design with the following operating characteristics. Power: 0.80; Type I error: 0.05; P0: 0.05; and P1: 0.25.

Each cohort enrolled 9 patients. If there was no complete (CR) or partial response (PR) in the first 9 patients the enrolment to that cohort was stopped. If there >1 PR or CR in the first 9 patients the enrollment continued to enroll total of 17 patients. The treatment was considered promising for further exploration if >3 CRs or PRs were observed in 17 patients. The expected Sample Size ranged from 9 (if terminated after safety lead in) to 34 patients. Accounted for ˜20% of patients not being evaluable for efficacy, the actual number of patients to be recruited for the trial ranged from 11 to 42.

TABLE 17
Operating characteristics:

| | Pr(reject | Pr(reject | Average | Average |
|---|---|---|---|---|
| Scenarios | H0 for | H0 for | sample size | sample size |
| (PA, PB) | cohort A) | cohort B) | for cohort A | for cohort B |
| (0.05, 0.05) | 0.046 | 0.046 | 12.0 | 12.0 |
| (0.05, 0.25) | 0.046 | 0.813 | 12.0 | 16.4 |
| (0.25, 0.25) | 0.813 | 0.813 | 16.4 | 16.4 |
| (0.05, 0.15) | 0.046 | 0.455 | 12.0 | 15.1 |
| (0.15, 0.15) | 0.455 | 0.455 | 15.1 | 15.1 |
| (0.15, 0.25) | 0.455 | 0.813 | 15.1 | 16.4 |

Note: PA was the response rate for cohort A, PB was the response rate for cohort B

The Investigator was responsible for completing toxicity/efficacy summary reports and submitting them to the IND office Medical Affairs and Safety Group for review. These should be submitted as follows:
Lead-In Stage:

After the first 6 evaluable patients, cycle 1 of study treatment was completed. IND Office approval was obtained prior to advancing to the efficacy stage.
Efficacy Stage:

After the first 9 evaluable patients per cohort, 9 weeks of study treatment was completed, and after a total of 17 patients per cohort 9 weeks of study treatment was completed.

Toxicity monitoring was also performed in this stage.

### Safety Analyses

All patients in the safety population were included in the final summaries and listings of safety data for the lead-in patients. Summaries of AEs and other safety parameters were provided as appropriate. Emphasis in the analysis of AEs was placed on those that were treatment-emergent through 30 days after last dose of Talabostat mesylate with pembrolizumab

Frequencies of patients experiencing at least 1 AE were displayed by body system and preferred term according to Medical Dictionary for Regulatory Activities (MedDRA) terminology. Detailed information collected for each AE included: a description of the event, duration, whether the AE was serious, nature of the event (single episode versus multiple episode), intensity (i.e., NCI CTCAE version 5 grade), relationship to study drug, action taken, clinical outcome, and whether the AE resulted in surgery or alternate procedures. Intensity (severity) of the AEs was graded according the NCI CTCAE. The latest version of MedDRA and NCI CTCAE was used.

Summary tables were prepared to show the number of patients reporting AEs, the frequency of patient reports, and corresponding percentages. Percentages were calculated using the number of patients in the safety population as the denominator. Within each table, the AEs were categorized by MedDRA body system and preferred term. Additional subcategories were based on event intensity and relationship to study drug. AE data was presented across all cycles and for each cycle. The denominator for each cycle was those patients available at the start of the cycle who received a dose of Talabostat mesylate for that cycle.

To the extent possible, AE relationship to either or pembrolizumab was identified.

Individual patient listings were prepared for all AE data. Vital signs and ECOG performance status were summarized by visits/cycles, using descriptive statistics applicable to continuous or categorical measures of these additional safety data. Summaries for the Lead-in and Efficacy Stages were presented.

TABLE 18
Study Schedule of Assessments

| | Screen | | | | | | | | | | | | | | | | | |
|---|---|---|---|---|---|---|---|---|---|---|---|---|---|---|---|---|---|---|
| | Period | | | | | | | | | | | | | | | | | |
| Screen/Cycle | D-28 to | | Cycle 1 | | | | | Cycle 2 | | | | | >Cycle 2 | | | | EOT | FU |
| Day (D): | D-1 | D1ⁱ | D2 | D5^{t} | D8^{r} | D14 | D15^{r} | D1^{c} | D2 | D8^{r} | D14 | D15^{r} | D1^{c} | D8 | D14 | D15 | Visit^{a} | Visit^{b} |
| Informed | X | | | | | | | | | | | | | | | | | |
| consent | | | | | | | | | | | | | | | | | | |
| Inclusion and | X | X | | | | | | | | | | | | | | | | |
| exclusion | | | | | | | | | | | | | | | | | | |
| criteria | | | | | | | | | | | | | | | | | | |
| Demographics | X | | | | | | | | | | | | | | | | | |
| Adverse Event | X | X | X | X | X | | X | X | | X | | X | X | X | | X | X | X |
| Assessment | | | | | | | | | | | | | | | | | | |
| Concomitant | X | X | X | X | X | | X | X | | X | | X | X | X | | X | X | X |
| Medications | | | | | | | | | | | | | | | | | | |
| cancer | X | | | | | | | | | | | | | | | | | |
| treatment | | | | | | | | | | | | | | | | | | |
| history | | | | | | | | | | | | | | | | | | |
| Archival tumor | X | | | | | | | | | | | | | | | | | |
| collection^{l} | | | | | | | | | | | | | | | | | | |
| Imaging and | | | | | | | | | | | | | | | | | | |
| Other | | | | | | | | | | | | | | | | | | |
| CT/MRI | X | | | | | | | | | | | | X^{d} | | | | X^{d} | |
| Tumor | X | | | | | | | | | | | | X^{d} | | | | X^{d} | |
| assessment | | | | | | | | | | | | | | | | | | |
| Study Drug | | | | | | | | | | | | | | | | | | |
| Administration | | | | | | | | | | | | | | | | | | |
| Talabostat | | | Day 1-14 | | | | | Day 1-14 | | | | | Day 1-14 | | | | | |
| administration | | | | | | | | | | | | | | | | | | |
| Ambulatory | | | Day 1-14 | | | | | Day 1-14 | | | | | Day 1-14 | | | | | |
| monitoring of | | | | | | | | | | | | | | | | | | |
| blood pressure | | | | | | | | | | | | | | | | | | |
| before each | | | | | | | | | | | | | | | | | | |
| dose of | | | | | | | | | | | | | | | | | | |
| Talabostat | | | | | | | | | | | | | | | | | | |
| Oral hydration | | | Day 1-14 | | | | | Day 1-14 | | | | | Day 1-14 | | | | | |
| monitoring | | | | | | | | | | | | | | | | | | |
| Daily safety | | | Day 3-4, Day 6-14 | | | | | | | | | | | | | | | |
| telephone calls | | | | | | | | | | | | | | | | | | |
| Pembrolizumab | | X | | | | | | X | | | | | X | | | | | |
| administration^{j} | | | | | | | | | | | | | | | | | | |
| Clinical | | | | | | | | | | | | | | | | | | |
| Procedures | | | | | | | | | | | | | | | | | | |
| Physical | X | X | X | X | X | | X | X | | X | | X | X | | | | X | |
| examination | | | | | | | | | | | | | | | | | | |
| Medical | X | X | X | X | X | | X | X | | X | | X | X | | | | X | |
| history/current | | | | | | | | | | | | | | | | | | |
| medical | | | | | | | | | | | | | | | | | | |
| conditions | | | | | | | | | | | | | | | | | | |
| ECOG | X | X | X | X | X | | X | X | | X | | X | X | | | | X | |
| performance | | | | | | | | | | | | | | | | | | |
| status | | | | | | | | | | | | | | | | | | |
| Vital signs (BP, | X | X | X | X | X | | X | X | | X | | X | X | | | | X | |
| HR, body | | | | | | | | | | | | | | | | | | |
| temperature, | | | | | | | | | | | | | | | | | | |
| RR, O2 | | | | | | | | | | | | | | | | | | |
| saturation) | | | | | | | | | | | | | | | | | | |
| Height | X | X | X | X | X | | X | X | | X | | X | X | | | | X | |
| Weight | X | X | X | X | X | | X | X | | X | | X | X | | | | X | |
| Clinical | | | | | | | | | | | | | | | | | | |
| Laboratory | | | | | | | | | | | | | | | | | | |
| Tests^{q} | | | | | | | | | | | | | | | | | | |
| Hematology | X | X | | | X | | X | X | | X | | X | X | X | | X | X | |
| (CBC plus | | | | | | | | | | | | | | | | | | |
| differential | | | | | | | | | | | | | | | | | | |
| [5-part or auto- | | | | | | | | | | | | | | | | | | |
| analyzer]) | | | | | | | | | | | | | | | | | | |
| Serum | X | X | | | X | | X | X | | X | | X | X | X | | X | X | |
| chemistry^{e} | | | | | | | | | | | | | | | | | | |
| Liver function | X | X | | | X | | X | X | | X | | X | X | X | | X | X | |
| tests^{f} | | | | | | | | | | | | | | | | | | |
| T3, FT4, TSH | Xu | | | | | | | | | | | | X^{u} | | | | X^{u} | |
| Cytokine-12 | | X | X | X | | | | | | | | | | | | | | |
| panel | | | | | | | | | | | | | | | | | | |
| Urinalysis | X | X | | | | | | X | | | | | X | | | | X | |
| Serum | | X | | | | | X | X | | | | X | | | | | | |
| collection^{g} | | | | | | | | | | | | | | | | | | |
| Whole blood | | X | | | | | X | X | | | | X | | | | | X | |
| collection for | | | | | | | | | | | | | | | | | | |
| immune | | | | | | | | | | | | | | | | | | |
| parameters^{k} | | | | | | | | | | | | | | | | | | |
| Tumor | X^{m} | | | | | | | | | X^{m} | | | | | | | X^{m} | |
| biopsies^{m} | | | | | | | | | | | | | | | | | | |
| Blood | X | X^{h} | X^{h} | | X^{h} | | X^{h} | X^{h} | | | | | X^{h} | | | | X | |
| collection for | | | | | | | | | | | | | | | | | | |
| ctDNA^{s} | | | | | | | | | | | | | | | | | | |
| Pregnancy test | Xⁿ | X^{o,p} | | | | | | X^{o} | | | | | X^{o} | | | | | |

Abbreviations: BP = blood pressure; CxDx = Cycle (number) Day (number); CBC = complete blood count; CT = computed tomography; ECOG = Eastern Cooperative Oncology Group; EOT = End of Treatment; FU = follow-up; HR = heart rate; MRI = magnetic resonance imaging; RR = respiratory rate.
^{a}After discontinuation of study drugs, patients completed an EOT visit within 21 days after the last study drug dose.
^{b}A Safety Follow-up Visit was to be conducted 30 days (±7 days) after the last dose of study drug and later if drug-related AEs had not resolved at that time. Thereafter, patients without documented disease progression (PD) were followed every 90 days for disease assessments until documentation of PD. After documentation of PD, patients were followed every 90 days for survival status; such follow-up was likely be conducted by telephone.
^{c}Day 1 of Cycle 2 and all subsequent cycles were 21 days (±3 days) after the previous dose of study drug was administered.
^{d}Tumor assessment must include cross-sectional imaging (MRI or CT scanning with intravenous contrast whenever possible) of the chest/abdomen/pelvis plus whole-body bone scan. Other body sites (e.g., neck) to be included as clinically indicated. Tumor assessment to be performed at Screening, C4D1 (±7 days), C7D1 (±7 days), C10D1 (±7 days), and Day 1 (±7 days) of every 3rd cycle thereafter and at EOT. Intervals for scans could be shortened to 6 weeks (e.g. C3D1, C5D1 etc.) if clinically necessary per treating physician.
^{e}Serum chemistry included: sodium (Na), potassium (K), chloride (Cl), bicarbonate, calcium (Ca), magnesium (Mg), phosphate, blood urea nitrogen (BUN)/creatinine (Cr), and lactate dehydrogenase (LDH).
^{f}Liver function tests included aspartate aminotransferase, alanine aminotransferase, alkaline phosphatase, total bilirubin, and albumin.
^{g}Serum was collected on predose (within an 1 hour ± 30 minutes) and then 6 hours (±1 hour) and 24 hours (±3 hours) postdose on C1D1 It was also collected on C1D15 (±1 days) and C2D1 (±1 day), and C2D15 (±1 day).
^{h}Samples should be collected before dosing.
ⁱIf Cycle 1 Day 1 occurs <72 hours after Screening, physical examinations and clinical laboratory tests do not need to be repeated.
^{j}Either Talabostat or pembrolizumab may be administered first. However, on Cycle 1 Day 1, it was recommended that pembrolizumab was administered first and that ≥1 hour should elapse before the administration of Talabostat mesylate so that it was easier to determine the relatedness of any AEs to study drug. It was recommended to administer at least 1 L of IV fluids if not medically contraindicated at Cycle 1 Day 1. It is at the investigator's discretion to provide IV hydration during in-person clinic visits beyond Cycle 1 Day 1.
^{k}Whole blood was be collected on C1D1, C1D15 (±1 day), C2D1, C2D15 (±1 day), and at EOT and for assessment of leukocytes by flow cytometry C1D1, C1D15 (±1 day), C2D1, C2D15 (±1 day), and at EOT and other studies
^{l}Archival tissue was collected if available.
^{m}Optional tumor biopsies were obtained during screening and between Day 8 and Day 14 of cycle2, and at EOT. Pre and post treatment biopsies were mandatory if medically feasible incohorts advancing to BOP2 stage
ⁿPregnancy tests were only for women with child bearing potential. Only whole blood was collected for the pregnancy test that was performed in the screening period (D-28 to D-1)
^{o}Additional pregnancy tests were performed D1 of each cycle only for women with child bearing potential. Whole blood or urine could be collected for the test.
^{p}Pregnancy test for the D1 of the 1^{st} cycle could be skipped if the test for the screening period was performed close to the 1^{st} cycle.
^{q}Laboratory assessment for immune-related toxicities were added as per patient's toxicity and as needed upon the investigator's discretion.
^{r}Days 8 and 15 of Cycles 1 and 2 could have a window of ±2 days. These visits could be also preformed using the telehealth in case of unanticipated events such as COVID-19 pandemics if in agreement with applicable regulations.
^{s}Details about collection and processing were in the Laboratory Manual.
^{t}C1D5 visit had a window of −1 day and can happen on C1D4.
^{u}T3, FT4 and TSH testing was performed at baseline, C4D1 (±7 days), C7D1 (±7 days), C10D1 (±7 days), and Day 1 (±7 days) of every 3rd cycle thereafter and at EOT.

### Results:

Patient Characteristics:

14 patients with median age of 63 years (in range 36-86 years) were treated including 9 males (64%) and 5 females (36%).

Four (4) patients (castrate resistant prostate cancer, endometrial cancer, dedifferentiated liposarcoma, basal cell carcinoma) were enrolled in the PD-1/PD-L1 or CTLA-4 naïve cohort A and ten (10) patients (leiomyosarcoma, squamous cell carcinoma of unknown primary, skin melanoma, pleomorphic sarcoma, colorectal/colon cancer, astrocytoma, TNBC, uterine sarcoma, uveal melanoma) were enrolled in PD-1/PD-L1 or CTLA-4 pretreated cohort B (see Table 19).

TABLE 19
showing the Patient characteristics (14 Patients enrolled till date)

| Characteristic | No. of Patients (%) |
|---|---|
| Age (years) | |
| Median (range) | 63(36-86) |
| Gender | |
| Male | 9 (64) |
| Female | 5 (36) |
| Cohort A (PD-1/PD-L1 or CTLA-4 naïve) | 4 |
| Cohort B (PD-1/PD-L1 or CTLA-4 pretreated) | 10 |
| Tumor type | |
| Castration resistant prostate cancer | 2 |
| Colon cancer | 1 |
| Endometrial cancer | 1 |
| Dedifferentiated liposarcoma | 1 |
| Basal cell carcinoma | 1 |
| Squamous cell carcinoma of unknown primary | 1 |
| Uveal melanoma | 1 |
| Leiomyosarcoma | 1 |
| Skin melanoma | 1 |
| Uterine sarcoma | 1 |
| TNBC | 1 |
| Pleomorphic sarcoma | 1 |
| Astrocytoma | 1 |

Safety:

Safety summary for Lead-in stage:

Among all 14 patients, there was 1 episode of grade 4 hypotension with syncope as the only DLT on Day 6 of cycle 1 related to Talabostat in a patient with endometrial cancer. The patient fully recovered with appropriate medical care.

Safety Summary for Efficacy Stage:
Grade 4 hypotension related to Talabostat with subsequent cardiac arrest and death on day 5 of cycle 1
Other adverse effects present in more than 5% of patients included manageable grade 1 or 2 edema, flu-like symptoms, fever, fatigue, nausea, vomiting and maculopapular rash.

Efficacy:

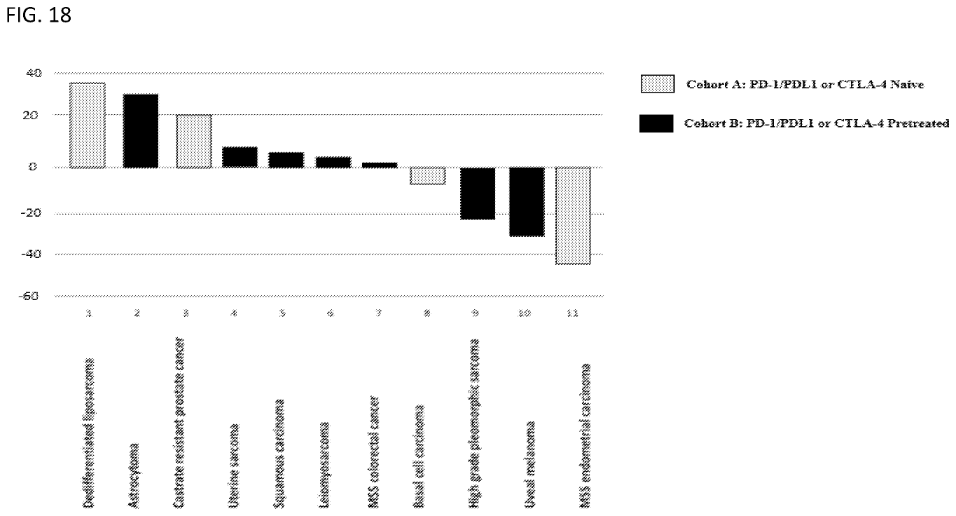

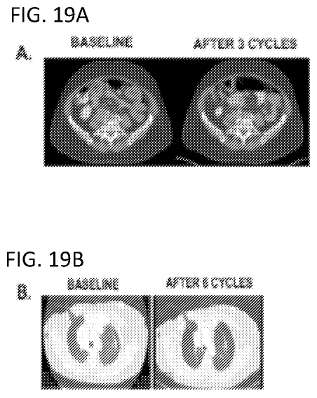

In the PD-1/PD-L1 and/or CTLA-4 naïve cohort (cohort A; n=4) 1 had a PR (−64%) in a patient with microsatellite stable (MSS) endometrial cancer, PD-L1 negative (CPS<1). 1 SD (−7%) in a patient with basal cell carcinoma, PD-L1 negative (see FIGS. 18 and 19A).

In the PD-1/PD-L1 and/or CTLA-4 pretreated cohort (cohort B; n=10), 1 had a PR (−31%) in a patient with uveal melanoma, PD-L1 negative. 1 SD (−23%) in a patient with pleomorphic sarcoma, MSS, PD-L1 negative. Additional SDs were observed in patients with squamous cell carcinoma of unknown primary (+4%) and uterine sarcoma (+5%) (FIGS. 18 and 19B).

Conclusions: Talabostat mesylate in combination with pembrolizumab demonstrated encouraging signals of activity in selected difficult-to-treat cancers in patients with and without prior treatment with PD-1/PD-L1 and/or CTLA-4 inhibitors. Mitigation strategies (e.g. intra-patient dose escalating dosing schedule for Talabostat mesylate; ambulatory blood pressure monitoring etc.) to prevent events of high-grade hypotension during the first week of therapy are being implemented as per protocol amendment.

1. A method of treating prostate cancer in a subject comprising administering talabostat or a pharmaceutically acceptable salt thereof in combination with a therapeutically effective amount of pembrolizumab, wherein talabostat or a pharmaceutically acceptable salt thereof is administered at a dose of about 0.3 mg twice daily on one or more days of a treatment cycle, and
wherein the prostate cancer is selected from the group consisting of small cell neuroendocrine prostate cancer (SCNC), neuroendocrine prostate cancer (NEM), treatment emergent neuroendocrine prostate cancer (tNEPC), castration resistant prostate cancer (CrPC), metastatic castration resistant prostate cancer (mCrPC), and adenocarcinoma type prostate cancer.

2. The method of claim 1, wherein the treatment cycle is of 21-days duration and talabostat or a pharmaceutically acceptable salt thereof is administered on each of days 1 to 14 and pembrolizumab is administered on day 1.

3. The method of claim 1, wherein the subject is administered talabostat or a pharmaceutically acceptable salt thereof at a dose of about 0.2 mg twice daily for one or more consecutive days beginning on day 1 of the first treatment cycle.

4. The method of claim 3, wherein the subject is administered talabostat or a pharmaceutically acceptable salt thereof at a dose of about 0.2 mg twice daily on days 1-7 of the first treatment cycle followed by about 0.3 mg twice daily on days 8-14 of the first treatment cycle.

5. The method of claim 1, comprising one or more additional treatment cycles.

6. The method of claim 1, wherein talabostat or a pharmaceutically acceptable salt thereof is administered orally in the morning and evening.

7. The method of claim 1, wherein talabostat or a pharmaceutically acceptable salt thereof is present as talabostat mesylate.

8. The method of claim 1, wherein pembrolizumab is administered intravenously at a total dose of about 200 mg.

9. The method of claim 1, wherein the prostate cancer is adenocarcinoma type prostate cancer.

10. The method of claim 1, wherein the subject experiences less treatment-related adverse events (TRAEs) relative to a subject with same cancer administered a single 0.6 mg daily dose of talabostat.

11. The method of claim 1, wherein the subject experiences no TRAEs.

12. The method of claim 10, wherein the TRAEs are one or more of hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, rhabdomyolysis myalgia, constipation, hypocalcemia, neutropenia, febrile neutropenia, anemia, leukopenia, pancytopenia, and lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest.

13. (canceled)

14. (canceled)

15. (canceled)

16. The method of claim 1, wherein the subject achieves a 50% or greater prostate-specific antigen (PSA) decline from baseline by week 12 of treatment.

17. The method of claim 1, wherein the subject experiences an increase in pro-inflammatory cytokines relative to a subject that is administered a single 0.6 mg daily dose of talabostat.

18. The method of claim 17, wherein the pro-inflammatory cytokines are one or more of IL-18 and IFN-γ.

19. The method of claim 17, wherein the maximum increase in cytokines is observed at day 14 of continuous dosing.

20. (canceled)

21. (canceled)

22. (canceled)

23. The method of claim 1, wherein the treatment cycle is a 21-day treatment cycle and talabostat or a pharmaceutically acceptable salt thereof is administered on each of days 1 to 14 and pembrolizumab is administered on day 1.

24. The method of claim 1, wherein the subject is administered talabostat or a pharmaceutically acceptable salt thereof at a dose of about 0.2 mg twice daily for one or more consecutive days beginning on day 1 of the first treatment cycle.

25. The method of claim 24, wherein the subject is administered talabostat or a pharmaceutically acceptable salt thereof at a dose of about 0.2 mg twice daily on days 1-7 of the first treatment cycle followed by about 0.3 mg twice daily on days 8-14 of the first treatment cycle.

26-39. (canceled)

40. A method of treating prostate cancer in a subject comprising administering talabostat or a pharmaceutically acceptable salt thereof and a therapeutically effective amount of pembrolizumab,
wherein talabostat or a pharmaceutically acceptable salt thereof is administered at a dose of about 0.2 mg one or more times daily on one or more days of a treatment cycle, and
wherein the prostate cancer is selected from the group consisting of small cell neuroendocrine prostate cancer (CNC), neuroendocrine prostate cancer (NEPC), treatment emergent neuroendocrine prostate cancer (tNEPC), castration resistant prostate cancer (CrPC), metastatic castration resistant prostate cancer (mCrPC), and adenocarcinoma type prostate cancer.
